(19) United States
(12) Patent Application Publication
(10) Pub. No.: US 2024/0161868 A1
(43) Pub. Date: May 16, 2024

(54) SYSTEM AND METHOD FOR GENE EXPRESSION AND TISSUE OF ORIGIN INFERENCE FROM CELL-FREE DNA

(71) Applicant: The Board of Trustees of the Leiand Stanford Junior University, Stanford, CA (US)

(72) Inventors: Maximilian Diehn, San Carlos, CA (US); Arash Ash Alizadeh, San Mateo, CA (US); Mahya Mehrmohamadi, Moraga, CA (US); Mohammad Shahrokh Esfahani, Mountain View, CA (US)

(21) Appl. No.: 17/980,254

(22) Filed: Nov. 3, 2022

Related U.S. Application Data
(63) Continuation of application No. PCT/US2021/032046, filed on May 12, 2021.
(60) Provisional application No. 63/023,728, filed on May 12, 2020.

(51) Int. Cl. G16B 25/10; C12N 15/10; C12Q 1/6813; C12Q 1/6869; G01N 33/574

(52) U.S. Cl. CPC G16B 25/10; C12N 15/1093; C12Q 1/6813; C12Q 1/6869; G01N 33/574

(57) ABSTRACT

Methods are provided for non-invasively determining the expression of genes of interest by inference and the use thereof in cancer classification and stratification for treatment. The methods are based on an integrated analytic method, where a single biomarker is derived from promoter fragment entropy (PFE) and analysis of nucleosome depleted regions (NDR) depth. In some embodiments the methods use only noninvasive blood draws, and robustly identify which patients will achieve durable clinical benefit from immune checkpoint inhibition, what the cancer subtype classification is and/or what the tumor burden is. In an embodiment, the methods further comprise selecting a treatment regimen for the individual based on the analysis.

### CROSS-REFERENCE To RELATED APPLICATIONS

The present application is a Continuation and claims the benefit of PCT Application No. PCT/US2021/032046, filed May 12, 2021, which claims the benefit of U.S. Provisional Patent Application No. 63/023,728 filed May 12, 2020, the entire disclosure of which is hereby incorporated by reference herein in their entireties for all purposes.

### STATEMENT REGARDING FEDERALLY SPONSORED RESEARCH

This invention was made with Government support under contract CA188298 awarded by the National Institutes of Health. The Government has certain rights in the invention.

### BACKGROUND OF THE INVENTION

Cell-free DNA (cfDNA) molecules that circulate in blood plasma largely arise from chromatin fragmentation accompanying cell death during homeostasis of diverse tissues throughout the body. Accordingly, cfDNA profiling has established clinical utility for detection of tissue rejection after solid organ transplantation, noninvasive prenatal testing of fetal aneusomies during pregnancy, and noninvasive tumor genotyping, as well as early evidence of utility for detection of diverse cancer types. For each of these applications, current liquid biopsy testing approaches have largely relied on germline or somatic genetic variations in the sequence of cfDNA molecules as relevant for diagnosis of pathology in the tissue of interest. Indeed such variations in genetic sequences can be highly informative for biopsy-free tumor genotyping of circulating tumor DNA (ctDNA) and for monitoring of disease burden, with potential utility for diagnosis and early cancer detection.

Despite the many applications of cfDNA profiling for the noninvasive detection of mutations in the blood, even in cancers with a high tumor mutation burden and even in patients with high disease burden, most cancer-derived fragments are generally unmutated. Accordingly, the ability to interrogate these cfDNA fragments to inform the tissue of origin of unmutated molecules using epigenetic features could have broad utility. For example, such approaches could be useful for detection of tissue injury without associated genetic lesions, as well as for classification of cancer entities and molecular subtypes. Since circulating cfDNA molecules are primarily nucleosome-associated fragments, they reflect the distinctive chromatin configuration of the nuclear genome of the cells from which they derived. Specifically, genomic regions densely associated with nucleosomal complexes are generally protected against the action of intracellular and extracellular endonucleases, while open chromatin regions are more exposed to such degradation.

Accordingly, several studies have recently identified specific chromatin fragmentation features across the genome as potentially useful for classification of tissue of origin by cfDNA profiling. These ‘fragmentomic’ features include a decrease in depth of sequencing coverage and disruption of nucleosome positioning near transcription start sites (TSSs). Separately, several studies have shown that the length of cfDNA fragments can also inform tissue of origin, including tumor derivation, even when considered agnostic to genomic location or relation to gene promoters. For example, tumor-derived molecules bearing somatic variants tend to be shorter than their wild-type counterparts and can be useful for distinguishing somatic variants that are tumor-derived from those arising from circulating leukocytes during clonal hematopoiesis.

Despite these advances, current fragmentomic methods, including those relying on relatively shallow whole genome sequencing (WGS) do not fully harness the contributions of various tissues to the circulating DNA pool. Separately, current fragmentomic techniques do not provide adequate genomic depth and breadth to enable gene-level resolution. Indeed, even when considering groups of genes, such fragmentomic methods only perform reasonably well for inferring gene expression at high circulating tumor DNA levels. Accordingly, fragmentomic methods for inferring gene expression are largely limited to patients with very high tumor burden generally observed in advanced disease.

### SUMMARY OF THE INVENTION

Compositions and methods are provided for non-invasively determining the expression of genes of interest by inference based on analysis of circulating cell-free DNA (cfDNA) in a sample of interest. In some embodiments the sample of interest is a noninvasive blood draw from a patient. In the methods, analysis of mRNA is not required for determining expression levels. The expression profile is useful, for example, in methods of prognosis and diagnosis. Methods of prognosis and diagnosis include, for example, determining whether an individual with cancer will have a durable clinical benefit from treatment with an immune checkpoint inhibitor, methods for determining whether an individual with non-small cell lung carcinoma (NSCLC) is classified as adenocarcinomas (LUAD) or squamous cell carcinomas (LUSC), methods for quantifying tumor burden in individuals living with diffuse large B cell lymphoma (DLBCL), methods for determining the cell of origin in individuals living with DLBCL, etc. In an embodiment, the methods further comprise selecting a treatment regimen for the individual based on the analysis. In some embodiments, the prediction is based on samples shortly after a first ICI treatment.

In an embodiment, an integrated analytic method is provided, where a single biomarker is derived from promoter fragment entropy (PFE) and analysis of nucleosome depleted regions (NDR) depth, each of which is calculated by sequencing of cfDNA from a sample of interest, e.g. a blood or blood-derived sample, at DNA regions flanking transcriptional start sites (TSS). A library is constructed from the cfDNA. The library is then contacted with oligonucleotide probes (i.e. a selector) that hybridizes to a sequence defined by the user (i.e. a TSS). The cfDNA can be enriched for TSS by hybrid-capture of these regions prior to sequencing. PFE is calculated by analyzing the range of fragmentation patterns of cfDNA at transcription start sites. NDR is calculated by analyzing the sequencing coverage from about −150 bp to +50 bp of the TSS. PFE and NDR, are independently associated with gene expression. Features that are associated with decreased gene expression are lower PFE; higher NDR, while decreased gene expression is associated with higher PFE and lower NDR. which is determined from sequencing cfDNA. NDR depth can be normalized to the specific DNA region being analyzed, which may be referred to as normalized NDR depth, and the resulting value integrated with PFE to provide a single predictive metric.

In some embodiments, a selector set may be used for the targeting of specific TSSs within the genome during hybrid capture prior to sequencing. In some embodiments, the selector set comprises selectors for one or more genes identified in Table 2. For instance, the selector set may comprise at least 10 selectors from Table 2, 50 selectors, 100 selectors, 150 selectors, 200 selectors or the complete list of selectors in Table 2, or may be a group as indicated in Table 2.

By integrating a measurement of PFE and NDR, i.e. normalized NDR depth, methods are provided for an entirely noninvasive multi-analyte assay (EPIC-seq, Expression Inference from Cell-free DNA Sequencing) that robustly predicts gene expression from a patient sample. The analysis may be implemented in hardware or software, or a combination of both. In one embodiment of the invention, a machine-readable storage medium is provided, the medium comprising a data storage material encoded with machine readable data which, when using a machine programmed with instructions for using said data, is capable of displaying a any of the datasets and data comparisons of this invention.

In other embodiments, the method is excuted through the use of a computer based software program wherein the PFE and NDR depth are inputed and the software program outputs a score indicative of a particular classification as defined by the user. The software programs employs machine learning to uncover relationships between input metrics in their relation to target outputs through training algorithms.

An individual for assessment by the method of the invention may have cancer. In some embodiments the individual has been previously diagnosed with the cancer. In some embodiments the cancer is a carcinoma, including without limitation non-small cell lung carcinoma, small cell lung carcinoma, adenocarcinoma, squamous cell carcinoma, hepatocarcinoma, basal cell carcinoma, etc., which may be breast cancer, colorectal cancer, bladder cancer, head and neck cancer, renal cell cancer, liver cancer, skin cancer, pancreatic cancer, etc. In some embodiments the cancer is a lymphoma, e.g. Hodgkin lymphoma, non-hodgkin lymphoma, etc. In some embodiments the cancer is a melanoma. In certain embodiments the individual has non-small cell lung cancer (NSCLC), which may be early stage, or advanced stage.

In some embodiments a method is provided of using EPIC-seq to facilitate personalized selection of treatment, including ICI if appropriate, for patients with a number of different cancers. When EPIC-seq is used to determine if an individual will receive DCB from ICI treatment, an individual with a low score that is predicted to benefit from ICI, can be selected, and treated, with an ICI, usually in combination with additional therapeutic agents. An individual with a high score that is not predicted to benefit from ICI can be selected, and treated, with non-ICI therapy, e.g. chemotherapy, non-ICI immunotherapy, radiation therapy, and the like. ICI of interest include, without limitation, inhibitors of PD-1 and inhibitors of PD-L1.

In some embodiments a method is provided of using EPIC-seq to facilitate cancer subtype classification for individuals with a cancer subtype of unknown origin i.e. an individual with NSCLC where it is unclear if it is LUAD or LUSC or an individual with DLBCL where it is unclear if it originated from the ABC or GBC. In one embodiment, when an individual is determined to have one cancer subtype and not another, i.e. the individual is diagnosed as LUAD and not LUSC, the individual may then by treated, as determined by a physician, for said cancer subtype. For instance, if an individual's cancer subtype was determined to be LUAD they may be treated with bevacizumab in combination with chemotherapy whereas if it was determined that the individual's cancer subtype was LUSC they may be treated with nectitumab in combination with cisplatin and gemcitabine.

In one embodiment, EPIC-seq facilitates personalized selection of therapy, which may include ICI, for patients with advanced cancers, to improve outcomes while minimizing toxicities. For example, patients with late stage disease can be treated with single-agent PD-1 blockade for one cycle irrespective of PD-L1 expression and then use EPIC-seq to determine the individual's response to treatment. Patients with low EPIC-seq scores (expected durable benefit) remain on single agent PD-1 blockade whereas patients with high EPIC-seq scores (expected lack of benefit) would receive treatment escalation through the addition of chemotherapy.

In other embodiments of the invention a device or kit is provided for the analysis of patient samples. Such devices or kits will include reagents that specifically identify one or more cells and signaling proteins indicative of the status of the patient, including without limitation affinity reagents. The reagents can be provided in isolated form, or pre-mixed as a cocktail suitable for the methods of the invention. A kit can include instructions for using the plurality of reagents to determine data from the sample; and instuctions for statistically analyzing the data. The kits may be provided in combination with a system for analysis, e.g. a system implemented on a computer. Such a system may include a software component configured for analysis of data obtained by the methods of the invention.

### BRIEF DESCRIPTION OF THE DRAWINGS

The invention is best understood from the following detailed description when read in conjunction with the accompanying drawings. The patent or application file contains at least one drawing executed in color. Copies of this patent or patent application publication with color drawing(s) will be provided by the Office upon request and payment of the necessary fee. It is emphasized that, according to common practice, the various features of the drawings are not to-scale. On the contrary, the dimensions of the various features are arbitrarily expanded or reduced for clarity. Included in the drawings are the following figures.

FIGS. 1A-1G. Correlation of gene expression and cell-free DNA molecular features. (FIG. 1A) Chromatin accessibility footprints can be traced back to the tissue of origin. Open chromatin is subject to nuclease digestion resulting in decreased sequencing coverage depth, measured by nucleosome depletion rate (NDR), and fragment length diversity, measured by promoter fragmentation entropy (PFE). In this cartoon, lung epithelial cells exhibit very low expression of MS4A1 (CD20) but high expression of NKX2-1 (TTF1). The cfDNA fragments of a lung cancer patient consist of normal primarily hematopoietic cfDNA fragments mixed with fragments derived from lung adenocarcinoma cells undergoing apoptosis. Because the lung epithelial cell compartment has a lower coverage (NDR) and higher fragment length diversity (PFE) for NKX2-1 fragments, the resulting mixture shows similar changes with the net effect dependent on the total amount of circulating tumor-derived fragments. B-cells, on the other hand, highly express MS4A1 (CD20) with a very low expression level of NKX2-1. Accordingly, the cfDNA fragments of a B-cell lymphoma patient consist of normal cfDNA fragments admixed with B-cell derived ctDNA with overrepresentation of MS4A1 resulting in lower coverage and higher diversity of cfDNA fragment length values at the transcription start site (TSS). (FIG. 1B) A heatmap depicts cfDNA fragment size densities at transcription start sites (TSS) across the genome in an exemplar plasma sample profiled by high-depth whole-genome sequencing (˜250×). The X-axis depicts cfDNA fragment size, while the rows of the heatmap capture fragment density as ordered by GEP in blood leukocytes assessed by RNA-Seq using transcripts per million (TPM, right). Each row corresponds to one meta-gene encompassing the TSSs of 10 genes when ranked by a reference PBMC expression vector. The data are normalized column-wise for each cfDNA fragment size bin. Corresponding PFE, NDR, and TPM levels are depicted for each bin in dot plots on the right. (FIG. 1C) A scatter plot depicts the relationship between plasma cfDNA PFE versus leukocyte RNA expression levels (TPM), as in panel (b). (FIG. 1D) Pearson correlations between individual cfDNA fragment features (PFE, NDR, OCF, WPS, and MDS) and leukocyte geneexpression levels; OCF: orientation-aware cfDNA fragmentation; WPS: windowed protection score; MDS: motif diversity score. The error bars depict the 95% confidence intervals resulted from bootstrap replicates (resampling with replacement of gene groups). (FIG. 1E) The correlation between leukocyte gene expression and each of two leading cfDNA features (PFE and NDR) as a function of distance to the TSS center. The orange curve shows the higher average correlation for cfDNA PFE than NDR's correlation at all distances from the TSS center. The dotted lines correspond to the concordance measure when evaluated on the shorn leukocyte DNA from a matched blood PBMC sample. (FIG. 1F) Effect of sequencing depth (X-axis) on the correlation of cfDNA PFE and NDR with gene expression (Y-axis). For each down-sampled depth, three replicates are generated, and the shaded area illustrates three standard deviation above and below the mean. (FIG. 1G) A heatmap of ‘PFE’ reflected in exons of select genes in five exemplar specimens (columns) from patients with advanced carcinomas of the lung and prostate or healthy adults, as profiled by deep whole-exome cfDNA sequencing. Depicted genes (rows) were selected based on expected expression patterns in small cell lung cancers (SCLC) and castrate resistant prostate cancer (CRPC). The two SCLC samples are from pre-treatment and progression time points of one patient (AF=23.4% and 37.8%, respectively), while the CRPC meta-profiles were originally profiled by Adalsteinsson et al. As expected, AR exhibits high PFE in the CRPC cases, while ASCL1, ISNM1 and SOX2 exhibit high PFE in the SCLC cases relative to healthy adults.

FIGS. 2A-2E. EPIC-Seq design and workflow. (FIG. 2A) The schema depicts the general workflow of EPIC-Seq, starting with cfDNA extraction from plasma, library preparation and capture of TSS of genes of interest, high-throughput sequencing of enriched regions, and finally, cfDNA fragmentation analysis followed by machine learning models for prediction of expression at each TSS and classification of the specimen. (FIG. 2B-FIG. 2C) The volcano plots depict differentially expressed genes, as informative for histological classification in non-small cell lung cancer subtypes (lung adenocarcinoma [LUAD] vs lung squamous cell carcinoma [LUSC] from the TCGA), and in cell of-origin classification of diffuse large B-cell lymphoma (ABC vs GCB from Schmitz et al.). Genes highlighted in colors other than grey were selected for TSS capture in EPIC-Seq, after censoring genes with high expression in blood leukocytes (see Methods). (FIG. 2D) NKX2-1, encoding TTF1, known to be highly expressed in NSCLC-LUAD tumors, exhibits significantly higher predicted expression in cfDNA of patients with LUAD by EPIC-Seq. (FIG. 2E) MS4A1, encoding CD20, known to be a marker of DLBCL tumors, exhibits significantly higher predicted expression in cfDNA of patients with DLBCL by EPIC-Seq. Box-and-whisker plots depict predicted expression levels in individual samples profiled by EPIC-Seq (dots), with boxes spanning the inter-quartile range; the median is horizontally marked with a line in each box, and whiskers span the 1.5 IQRs in each patient cohort.

FIGS. 3A-3I. Application of EPIC-Seq for lung cancer detection and histological classification. (FIG. 3A) Receiver-Operator Curve (ROC) capturing performance of the EPIC-Lung classifier for distinguishing lung cancers from others in leave-one-batch-out analyses (AUC=0.91). The 95% confidence interval of the AUC is calculated using 2000 bootstrap replicates. (FIG. 3B) Relationship between EPIC-Lung scores and NSCLC disease Stage, with test for trend measured by Jonckheere's test (P=0.08). Box-and-whisker plots depict the EPIC-lung classifier score in individual samples profiled by EPIC-Seq (dots), with boxes spanning the inter quartile range; the median is horizontally marked with a line in each box, and whiskers span the 1.5 IQRs in each disease stage group. (FIG. 3C) Sensitivity analysis of the EPIC-Lung classifier at 95% specificity. Patients are grouped based on bins of mean circulating tumor allele fraction (<1%, 1-5% and >5%), estimated by CAPP-Seq on the same samples. Sensitivity improves as ctDNA AF increases with ˜33% of patients detectable when AF<1%. The error bars depict the 95% confidence interval of the sensitivity values resulted from 500 bootstrap replicates. (FIG. 3D) ROC curve of the LUAD vs LUSC classifier when tested in a leave-one-out framework (AUC=0.90, 95%-CI [0.83-0.97]). (FIG. 3E) Coefficients of the NSCLC histology classifier, with positive and negative coefficients favoring LUAD and LUSC, respectively. The coefficients are significantly associated with prior knowledge when comparing their magnitude and polarity by t test (P=0.033). Box-and-whisker plots are defined as in (b) and are resulted from 67 coefficient sets from classifiers trained in the leave-one-out cross-validation step. (FIG. 3F) Accuracy of the histology classifier as a function of tumor ctDNA fraction as measured by CAPP-Seq. The (optimal) threshold for classification is determined in the leave-one-out framework by minimizing the average of class-conditional errors. The error bars are defined as in (a). (FIG. 3G) Application of inferred gene expression values from EPIC-Seq in predicting response to immune-checkpoint inhibitors within 4 weeks of treatment initiation. (FIG. 3H) The scatterplot depicts change in an EPIC Seq lung dynamics score vs ctDNA response measured by CAPP-Seq; the latter calculated as log-transformed fold change of on-treatment to pre-treatment ctDNA concentration. The two orthogonal measures show a significant correlation (r=0.77, P=0.006). (FIG. 3I) ROC curve of the EPIC-Seq lung dynamics score calculated in panel g distinguishes patients with durable clinical benefit (DCB) vs those with no durable benefit (NDB) within the first 6 months (AUC=0.93, 95% CI [0.78-1]).

FIGS. 4A-4E. Application of EPIC-Seq for DLBCL detection. (FIG. 4A) Receiver-Operator Curve (ROC) capturing performance of the EPIC-DLBCL classifier for distinguishing lymphomas from others in leave-one-batch-out analyses (AUC=0.92). (FIG. 4B) Relationship between EPIC-Seq DLBCL classifier scores and clinical prognostic scores as measured by the Revised International Prognostic Index (R-IPI; Jonckheere's trend test P=4E−4). Box-and-whisker plots depict the EPIC-DLBCL score in individual samples profiled by EPIC-Seq (dots), with boxes spanning the inter-quartile range; the median is horizontally marked with a line in each box, and whiskers span the 1.5 IQRs. (FIG. 4C) Sensitivity analysis at 95% specificity for EPIC-DLBCL classifier. Similar to the EPIC-Lung cancer classifier, sensitivity significantly improves from ˜40% in cases with AF<1% to >95% for cases with AF>5%. The error bars depict the 95% confidence interval of the sensitivity values resulted from 500 bootstrap replicates. (FIG. 4D-FIG. 4E) Change of ctDNA disease burden in response to treatment and during clinical progression in two DLBCL patients with GCB (d) and ABC (e) cell-of-origin. Shown is the radiographic response as measured by PET/CT MTV (first row y-axis), ctDNA mean AF measured by CAPP-Seq (second row y-axis), and the EPIC seq lymphoma score (third row y-axis) over serial, pre- and post-therapy time points (x-axis).

FIGS. 5A-5E. Application of EPIC-Seq for DLBCL cell-of-origin classification. (FIG. 5A) Relationship between DLBCL cell-of-origin EPIC-Seq GCB scores and mutation-based GCB scores as measured by CAPP-Seq (Spearman rho=0.75, P=1 e−5). Data were smoothed by 3-patient bins after sorting by CAPP-Seq scores before correlation analysis. (FIG. 5B) Relationship between EPIC Seq GCB scores from cfDNA and tumor tissue clinical classification by Hans immunohistochemical algorithm (Wilcoxon P-value=0.001). Box-and-whisker plots depict the EPIC-Seq GCB score in individual samples profiled by EPIC-Seq (dots), with boxes spanning the inter-quartile range; the median is horizontally marked with a line in each box, and whiskers span the 1.5 IQRs. (FIG. 5C) Prognostic value of EPIC-Seq cell-of-origin scores in Kaplan-Meier analysis of Event Free Survival in DLBCL (log-rank P-value=0.013). Patients are stratified by the median EPIC-COO score, with higher scores associated with GCB and lower levels with ABC subtype. (FIG. 5D) Prognostic value of individual genes profiled by EPIC-Seq and Event-Free Survival, as measured by Z-scores from univariate Cox proportional hazard models. For genes with multiple TSS regions, Z-scores were combined using Stouffer's method. After correcting for multiple hypothesis testing, only LMO2 (red) remains significant significantly associated with favorable DLBCL outcome. Dotted lines represent the significance threshold for Bonferroni corrected P-values of 0.05. (FIG. 5E) Forest-plot depicts multivariable Cox proportional hazard model results for event-free survival (EFS). After adjusting for IPI and ctDNA allele fraction, only the distal TSS for LMO2 remains significantly prognostic for EFS (P=0.005).

FIGS. 6A-6D. Fragment length density at the transcription start sites varies with gene expression. (FIG. 6A) A heatmap of fragment length densities across 1,748 groups of genes (similar to FIG. 1a). Three regions R1 (100-150 bps), R2 (151-210 bps), and R3 (211-300 bps) show enrichment in either high or low expression gene groups. (FIG. 6B) The percent of fragments within each region defined in panel (FIG. 6A) in the deep whole-genome sample across deciles of the reference PBMC gene expression vector, i.e., 10 groups of genes when sorted by their expression values in PBMC. Highly expressed genes include fewer monosome fragments, indicating a wider distribution and thereby a higher PFE. (FIG. 6C) Fraction of fragments within the three regions, R1-R3, for exons vs introns vs TSS sites for the top (and bottom) 2000 genes as ranked by expression. The fraction of monosomal fragments within TSS regions is substantially lower than within intronic and exonic regions (63.5% at TSS vs ˜71% at non-TSS). Pearson's Chi-Squared goodness-of-fit tests resulted in the following test statistics (TSS vs Exon: G=62,133 [P<2.2E−16]; TSS vs Intron: G=84,110 [P<2.2E−16]). (FIG. D) The contour plot of the expression (depicted by heat) vs two features used in the gene inference model: PFE and NDR.

FIGS. 7A-7F. Ensemble model accurately predicts gene expression in validation samples. (FIG. 7A) The scatterplot of the predicted vs a population-averaged gene expression across 1,748 groups of genes. The underlying sample is a merged meta-sample (27 healthy subject in silico merged into one), achieving a correlation of 0.9 in validation. (FIG. 7B) The meta sample from panel (FIG. 7A) is used to assess the model performance when considering TSS level expression values without gene grouping, as well as scenarios with 2, 3, 5 and 10 genes per group. The Pearson correlation between model predicted expression and the PBMC expression is shown in green bars. This correlation substantially improves as number of genes per group increases. The correlation values between NDR and expression are shown in blue bars. (FIG. 7C-FIG. 7D) The same analysis as in panels (FIG. 7A-FIG. 7B) for a meta whole genome sample generated from healthy subjects from Zviran et al. (FIG. 7E) The whole genome samples (depth ˜20-40×) from Zviran et al. were used with every ten genes grouped and the concordance between model-predicted expression and PBMC expression are evaluated using Pearson correlation (i.e., each dot is one subject). The non-cancer samples show a higher correlation with normal PBMC than lung cancer cases with a Wilcoxon P-value of 0.018. (FIG. 7F) The ichorCNA tumor fraction estimates of the lung cancer cases in panel f are used to compare with the correlations in panel f. As shown in a scatterplot, as tumor fraction increases, the correlation decreases (r=−0.69, P=0.00052).

FIGS. 8A-8B. Cell-free DNA Samples profiled by EPIC-seq.

FIGS. 9A-9B. Concordance between EPIC-lung scores and clinical factors. (FIG. 9A) The concordance between EPIC-lung score and metabolic tumor volume (MTV). The two factors are evaluated using Spearman correlation. The correlation coefficient is ρ=0.67 with P-value of 0.04. (FIG. 9B) The concordance between EPIC-lung score and the ctDNA mean allele fractions is evaluated using Spearman correlation. The correlation coefficient is ρ=0.5 with P-value of 3E−5.

FIGS. 10A-10E. Concordance between EPIC-DLBCL scores and clinical factors and.

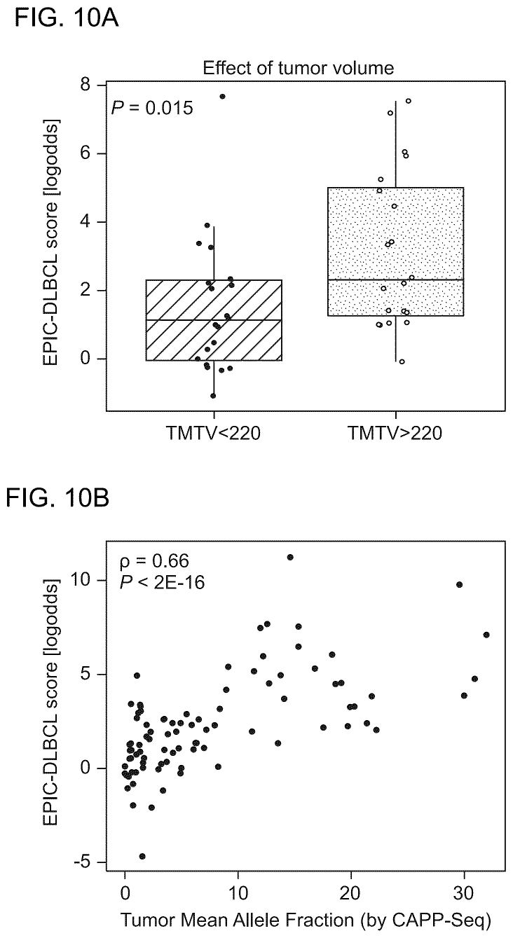

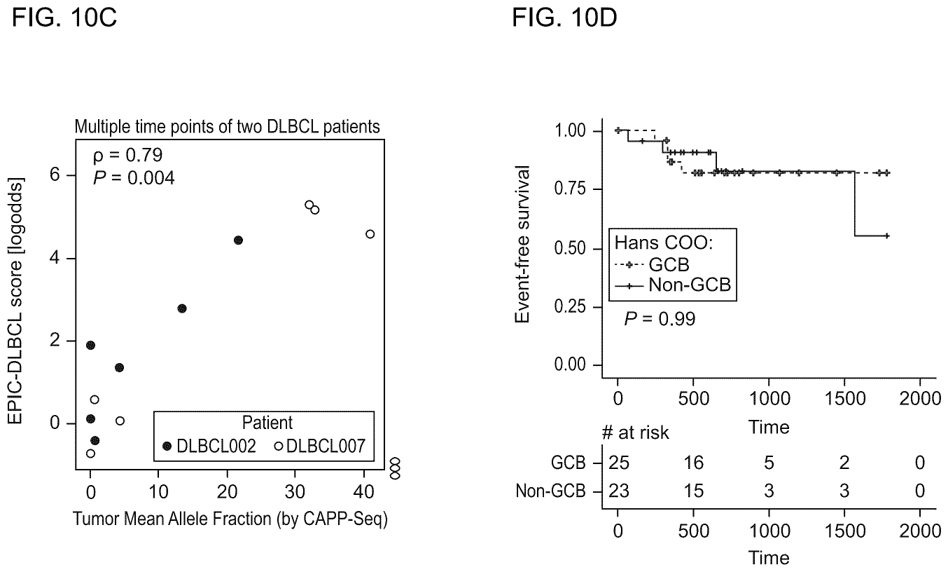

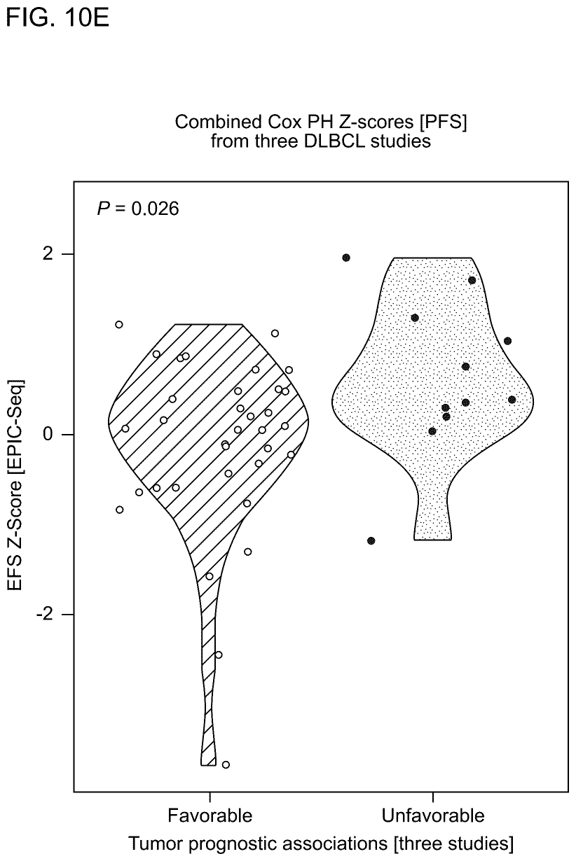

(FIG. 10A) The boxplots illustrate the two groups of patients stratified by their metabolic tumor volumes (>220 vs <220 mL). This analysis shows that the EPIC-DLBCL score is significantly higher in the ‘MTV>220’ group with a Wilcoxon P-value of 0.015. (FIG. 10B) The concordance between EPIC86 DLBCL scores and ctDNA mean allele fractions (from CAPP-Seq) is evaluated using Spearman correlation. The correlation coefficient is 0.66 with a P-value P<2E−16. (FIG. 10C) The EPIC-DLBCL model is applied to the cfDNA profiles of 13 samples from twfo DLBCL patients (DLBCL002 [ABC] and DLBCL007 [GCB]). The concordance between the resulting scores and the ctDNA mean allele fractions is evaluated by Spearman correlation. The correlation coefficient is 0.79 with a P-value of 0.004. (FIG. 10D) The Kaplan-Meier curves of EFS of the patients when labeled by the Hans algorithm. The non-GCB group contains both Non-GCB and Unknown. (FIG. 10E) The violin plot shows the distributions of Cox Proportional Hazard model Z-scores when genes are grouped according to their effects on outcome (measured as EFS) in three tumor studies.

### DETAILED DESCRIPTION

These and other features of the present teachings will become more apparent from the description herein. While the present teachings are described in conjunction with various embodiments, it is not intended that the present teachings be limited to such embodiments. On the contrary, the present teachings encompass various alternatives, modifications, and equivalents, as will be appreciated by those of skill in the art.

Most of the words used in this specification have the meaning that would be attributed to those words by one skilled in the art. Words specifically defined in the specification have the meaning provided in the context of the present teachings as a whole, and as are typically understood by those skilled in the art. In the event that a conflict arises between an art-understood definition of a word or phrase and a definition of the word or phrase as specifically taught in this specification, the specification shall control.

It must be noted that, as used in the specification and the appended claims, the singular forms “a,” “an,” and “the” include plural referents unless the context clearly dictates otherwise.

All publications, patents, and patent applications mentioned in this specification are herein incorporated by reference to the same extent as if each individual publication, patent, or patent application was specifically and individually indicated to be incorporated by reference.

The term “immune checkpoint inhibitor” refers to a molecule, compound, or composition that binds to an immune checkpoint protein and blocks its activity and/or inhibits the function of the immune regulatory cell expressing the immune checkpoint protein that it binds (e.g., Treg cells, tumor-associated macrophages, etc.). Immune checkpoint proteins may include, but are not limited to, CTLA4 (Cytotoxic T-Lymphocyte-Associated protein 4, CD152), PD1 (also known as PD-1; Programmed Death 1 receptor), PD-L1, PD-L2, LAG-3 (Lymphocyte Activation Gene-3), OX40, A2AR (Adenosine A2A receptor), B7-H3 (CD276), B7-H4 (VTCN1), BTLA (B and T Lymphocyte Attenuator, CD272), IDO (Indoleamine 2,3-dioxygenase), KIR (Killer-cell Immunoglobulin-like Receptor), TIM 3 (T-cell Immunoglobulin domain and Mucin domain 3), VISTA (V-domain Ig suppressor of T cell activation), and IL-2R (interleukin-2 receptor).

Immune checkpoint inhibitors are well known in the art and are commercially or clinically available. These include but are not limited to antibodies that inhibit immune checkpoint proteins. Illustrative examples of checkpoint inhibitors, referenced by their target immune checkpoint protein, are provided as follows. Immune checkpoint inhibitors comprising a CTLA-4 inhibitor include, but are not limited to, tremelimumab, and ipilimumab (marketed as Yervoy).

Immune checkpoint inhibitors comprising a PD-1 inhibitor include, but are not limited to, nivolumab (Opdivo), pidilizumab (CureTech), AMP-514 (Medlmmune), pembrolizumab (Keytruda), AUNP 12 (peptide, Aurigene and Pierre), Cemiplimab (Libtayo). Immune checkpoint inhibitors comprising a PD-L1 inhibitor include, but are not limited to, BMS-936559/MDX-1105 (Bristol-Myers Squibb), MPDL3280A (Genentech), MEDI 4736 (Medlmmune), MSB0010718C (EMD Sereno), Atezolizumab (Tecentriq), Avelumab (Bavencio), Durvalumab (Imfinzi).

Immune checkpoint inhibitors comprising a B7-H3 inhibitor include, but are not limited to, MGA271 (Macrogenics). Immune checkpoint inhibitors comprising an LAGS inhibitor include, but are not limited to, IMP321 (Immuntep), BMS-986016 (Bristol-Myers Squibb). Immune checkpoint inhibitors comprising a KIR inhibitor include, but are not limited to, IPH2101 (lirilumab, Bristol-Myers Squibb). Immune checkpoint inhibitors comprising an OX40 inhibitor include, but are not limited to MEDI-6469 (Medlmmune). An immune checkpoint inhibitor targeting IL-2R, for preferentially depleting Treg cells (e.g., FoxP-3+CD4+cells), comprises IL-2-toxin fusion proteins, which include, but are not limited to, denileukin diftitox (Ontak; Eisai).

The types of cancer that can be treated using the subject methods of the present invention include but are not limited to adrenal cortical cancer, anal cancer, aplastic anemia, bile duct cancer, bladder cancer, bone cancer, bone metastasis, brain cancers, central nervous system (CNS) cancers, peripheral nervous system (PNS) cancers, breast cancer, cervical cancer, childhood Non-Hodgkin's lymphoma, colon and rectum cancer, endometrial cancer, esophagus cancer, Ewing's family of tumors (e.g. Ewing's sarcoma), eye cancer, gallbladder cancer, gastrointestinal carcinoid tumors, gastrointestinal stromal tumors, gestational trophoblastic disease, hairy cell leukemia, Hodgkin's lymphoma, Kaposi's sarcoma, kidney cancer, laryngeal and hypopharyngeal cancer, acute lymphocytic leukemia, acute myeloid leukemia, children's leukemia, chronic lymphocytic leukemia, chronic myeloid leukemia, liver cancer, lung cancer, lung carcinoid tumors, Non-Hodgkin's lymphoma, male breast cancer, malignant mesothelioma, multiple myeloma, myelodysplastic syndrome, myeloproliferative disorders, nasal cavity and paranasal cancer, nasopharyngeal cancer, neuroblastoma, oral cavity and oropharyngeal cancer, osteosarcoma, ovarian cancer, pancreatic cancer, penile cancer, pituitary tumor, prostate cancer, retinoblastoma, rhabdomyosarcoma, salivary gland cancer, sarcomas, melanoma skin cancer, non-melanoma skin cancers, stomach cancer, testicular cancer, thymus cancer, thyroid cancer, uterine cancer (e.g. uterine sarcoma), transitional cell carcinoma, vaginal cancer, vulvar cancer, mesothelioma, squamous cell or epidermoid carcinoma, bronchial adenoma, choriocarinoma, head and neck cancers, teratocarcinoma, or Waldenstrom's macroglobulinemia.

Dosage and frequency may vary depending on the half-life of the agent in the patient. It will be understood by one of skill in the art that such guidelines will be adjusted for the molecular weight of the active agent, the clearance from the blood, the mode of administration, and other pharmacokinetic parameters. The dosage may also be varied for localized administration, e.g. intranasal, inhalation, etc., or for systemic administration, e.g. i.m., i.p., i.v., oral, and the like.

The terms “subject,” “individual,” and “patient” are used interchangeably herein to refer to a vertebrate, preferably a mammal, more preferably a human. Mammalian species that provide samples for analysis include canines; felines; equines; bovines; ovines; etc. and primates, particularly humans. Animal models, particularly small mammals, e.g. murine, lagomorpha, etc. can be used for experimental investigations. The methods of the invention can be applied for veterinary purposes.

As used herein, the term “theranosis” refers to the use of results obtained from a diagnostic method to direct the selection of, maintenance of, or changes to a therapeutic regimen, including but not limited to the choice of one or more therapeutic agents, changes in dose level, changes in dose schedule, changes in mode of administration, and changes in formulation. Diagnostic methods used to inform a theranosis can include any that provides information on the state of a disease, condition, or symptom.

The terms “therapeutic agent”, “therapeutic capable agent” or “treatment agent” are used interchangeably and refer to a molecule or compound that confers some beneficial effect upon administration to a subject. The beneficial effect includes enablement of diagnostic determinations; amelioration of a disease, symptom, disorder, or pathological condition; reducing or preventing the onset of a disease, symptom, disorder or condition; and generally counteracting a disease, symptom, disorder or pathological condition.

Non-ICI cancer therapy may include Abitrexate (Methotrexate Injection), Abraxane (Paclitaxel Injection), Adcetris (Brentuximab Vedotin Injection), Adriamycin (Doxorubicin), Adrucil Injection (5-FU (fluorouracil)), Afinitor (Everolimus) , Afinitor Disperz (Everolimus), Alimta (PEMET EXED), Alkeran Injection (Melphalan Injection), Alkeran Tablets (Melphalan), Aredia (Pamidronate), Arimidex (Anastrozole), Aromasin (Exemestane), Arranon (Nelarabine), Arzerra (Ofatumumab Injection), Avastin (Bevacizumab), Bexxar (Tositumomab), BiCNU (Carmustine), Blenoxane (Bleomycin), Bosulif (Bosutinib), Busulfex Injection (Busulfan Injection), Campath (Alemtuzumab), Camptosar (Irinotecan), Caprelsa (Vandetanib), Casodex (Bicalutamide), CeeNU (Lomustine), CeeNU Dose Pack (Lomustine), Cerubidine (Daunorubicin), Clolar (Clofarabine Injection), Cometriq (Cabozantinib), Cosmegen (Dactinomycin), CytosarU (Cytarabine), Cytoxan (Cytoxan), Cytoxan Injection (Cyclophosphamide Injection), Dacogen (Decitabine), DaunoXome (Daunorubicin Lipid Complex Injection), Decadron (Dexamethasone), DepoCyt (Cytarabine Lipid Complex Injection), Dexamethasone I ntensol (Dexamethasone), Dexpak Taperpak (Dexamethasone), Docefrez (Docetaxel), Doxil (Doxorubicin Lipid Complex Injection), Droxia (Hydroxyurea), DTIC (Decarbazine), Eligard (Leuprolide), Ellence (Ellence (epirubicin)), Eloxatin (Eloxatin (oxaliplatin)), Elspar (Asparaginase), Emcyt (Estramustine), Erbitux (Cetuximab), Erivedge (Vismodegib), Erwinaze (Asparaginase Erwinia chrysanthemi), Ethyol (Amifostine), Etopophos (Etoposide Injection), Eulexin (Flutamide), Fareston (Toremifene), Faslodex (Fulvestrant), Femara (Letrozole), Firmagon (Degarelix Injection), Fludara (Fludarabine), Folex (Methotrexate Injection), Folotyn (Pralatrexate Injection), FUDR (FUDR (floxuridine)), Gemzar (Gemcitabine), Gilotrif (Afatinib), Gleevec (Imatinib Mesylate), Gliadel Wafer (Carmustine wafer), Halaven (Eribulin Injection), Herceptin (Trastuzumab), Hexalen (Altretamine), Hycamtin (Topotecan), Hycamtin (Topotecan), Hydrea (Hydroxyurea), Iclusig (Ponatinib), Idamycin PFS (Idarubicin), Ifex (Ifosfamide), Inlyta (Axitinib), Intron A alfab (Interferon alfa-2a), Iressa (Gefitinib), Istodax (Romidepsin Injection), Ixempra (Ixabepilone Injection), Jakafi (Ruxolitinib), Jevtana (Cabazitaxel Injection), Kadcyla (Ado-trastuzumab Emtansine), Kyprolis (Carfilzomib), Leukeran (Chlorambucil), Leukine (Sargramostim), Leustatin (Cladribine), Lupron (Leuprolide), Lupron Depot (Leuprolide), Lupron DepotPED (Leuprolide), Lysodren (Mitotane), Marqibo Kit (Vincristine Lipid Complex Injection), Matulane (Procarbazine), Megace (Megestrol), Mekinist (Trametinib), Mesnex (Mesna), Mesnex (Mesna Injection), Metastron (Strontium-89 Chloride), Mexate (Methotrexate Injection), Mustargen (Mechlorethamine), Mutamycin (Mitomycin), Myleran (Busulfan), Mylotarg (Gemtuzumab Ozogamicin), Navelbine (Vinorelbine), Neosar Injection (Cyclophosphamide Injection), Neulasta (filgrastim), Neulasta (pegfilgrastim), Neupogen (filgrastim), Nexavar (Sorafenib), Nilandron (Nilandron (nilutamide)), Nipent (Pentostatin), Nolvadex (Tamoxifen), Novantrone (Mitoxantrone), Oncaspar (Pegaspargase), Oncovin (Vincristine), Ontak (Denileukin Diftitox), Onxol (Paclitaxel Injection), Panretin (Alitretinoin), Paraplatin (Carboplatin), Perjeta (Pertuzumab Injection), Platinol (Cisplatin), Platinol (Cisplatin Injection), PlatinolAQ (Cisplatin), PlatinolAQ (Cisplatin Injection), Pomalyst (Pomalidomide), Prednisone Intensol (Prednisone), Proleukin (Aldesleukin), Purinethol (Mercaptopurine), R-CHOP (Rituximab, Cyclophosphamide, Doxorubicin Hydrochloride {Hydroxydaunomycin}, Vincristine Sulfate {Onocvin} and Prednisone), Reclast (Zoledronic acid), Revlimid (Lenalidomide), Rheumatrex (Methotrexate), Rituxan (Rituximab), RoferonA alfaa (Interferon alfa-2a), Rubex (Doxorubicin), Sandostatin (Octreotide), Sandostatin LAR Depot (Octreotide), Soltamox (Tamoxifen), Sprycel (Dasatinib), Sterapred (Prednisone), Sterapred DS (Prednisone), Stivarga (Regorafenib), Supprelin LA (Histrelin Implant), Sutent (Sunitinib), Sylatron (Peginterferon Alfa-2b Injection (Sylatron)), Synribo (Omacetaxine Injection), Tabloid (Thioguanine), Taflinar (Dabrafenib), Tarceva (Erlotinib), Targretin Capsules (Bexarotene), Tasigna (Decarbazine), Taxol (Paclitaxel Injection), Taxotere (Docetaxel), Temodar (Temozolomide), Temodar (Temozolomide Injection), Tepadina (Thiotepa), Thalomid (Thalidomide), TheraCys BCG (BCG), Thioplex (Thiotepa), TICE BCG (BCG), Toposar (Etoposide Injection), Torisel (Temsirolimus), Treanda (Bendamustine hydrochloride), Trelstar (Triptorelin Injection), Trexall (Methotrexate), Trisenox (Arsenic trioxide), Tykerb (lapatinib), Valstar (Valrubicin Intravesical), Vantas (Histrelin Implant), Vectibix (Panitumumab), Velban (Vinblastine), Velcade (Bortezomib), Vepesid (Etoposide), Vepesid (Etoposide Injection), Vesanoid (Tretinoin), Vidaza (Azacitidine), Vincasar PFS (Vincristine), Vincrex (Vincristine), Votrient (Pazopanib), Vumon (Teniposide), Wellcovorin IV (Leucovorin Injection), Xalkori (Crizotinib), Xeloda (Capecitabine), Xtandi (Enzalutamide), Yervoy (Ipilimumab Injection), Zaltrap (Ziv-aflibercept Injection), Zanosar (Streptozocin), Zelboraf (Vemurafenib), Zevalin (Ibritumomab Tiuxetan), Zoladex (Goserelin), Zolinza (Vorinostat), Zometa (Zoledronic acid), Zortress (Everolimus), Zytiga (Abiraterone).

Radiotherapy means the use of radiation, usually X-rays, to treat illness. X-rays were discovered in 1895 and since then radiation has been used in medicine for diagnosis and investigation (X-rays) and treatment (radiotherapy). Radiotherapy may be from outside the body as external radiotherapy, using X-rays, cobalt irradiation, electrons, and more rarely other particles such as protons. It may also be from within the body as internal radiotherapy, which uses radioactive metals or liquids (isotopes) to treat cancer.

As used herein, “treatment” or “treating,” or “palliating” or “ameliorating” are used interchangeably. These terms refer to an approach for obtaining beneficial or desired results including but not limited to a therapeutic benefit and/or a prophylactic benefit. By therapeutic benefit is meant any therapeutically relevant improvement in or effect on one or more diseases, conditions, or symptoms under treatment. For prophylactic benefit, the compositions may be administered to a subject at risk of developing a particular disease, condition, or symptom, or to a subject reporting one or more of the physiological symptoms of a disease, even though the disease, condition, or symptom may not have yet been manifested.

The term “effective amount” or “therapeutically effective amount” refers to the amount of an agent that is sufficient to effect beneficial or desired results. The therapeutically effective amount will vary depending upon the subject and disease condition being treated, the weight and age of the subject, the severity of the disease condition, the manner of administration and the like, which can readily be determined by one of ordinary skill in the art. The term also applies to a dose that will provide an image for detection by any one of the imaging methods described herein. The specific dose will vary depending on the particular agent chosen, the dosing regimen to be followed, whether it is administered in combination with other compounds, timing of administration, the tissue to be imaged, and the physical delivery system in which it is carried.

“Suitable conditions” shall have a meaning dependent on the context in which this term is used. That is, when used in connection with an antibody, the term shall mean conditions that permit an antibody to bind to its corresponding antigen. When used in connection with contacting an agent to a cell, this term shall mean conditions that permit an agent capable of doing so to enter a cell and perform its intended function. In one embodiment, the term “suitable conditions” as used herein means physiological conditions.

The term “inflammatory” response is the development of a humoral (antibody mediated) and/or a cellular response, which cellular response may be mediated by antigen-specific T cells or their secretion products), and innate immune cells. An “immunogen” is capable of inducing an immunological response against itself on administration to a mammal or due to autoimmune disease.

The terms “biomarker,” “biomarkers,” “marker” or “markers” for the purposes of the invention refer to, without limitation, proteins together with their related metabolites, mutations, variants, polymorphisms, modifications, fragments, subunits, degradation products, elements, and other analytes or sample-derived measures. Markers can include expression levels of an intracellular protein or extracellular protein. Markers can also include combinations of any one or more of the foregoing measurements, including temporal trends and differences. Broadly used, a marker can also refer to an immune cell subset.

To “analyze” includes determining a set of values associated with a sample by measurement of a marker (such as, e.g., presence or absence of a marker or constituent expression levels) in the sample and comparing the measurement against measurement in a sample or set of samples from the same subject or other control subject(s). The markers of the present teachings can be analyzed by any of various conventional methods known in the art. To “analyze” can include performing a statistical analysis, e.g. normalization of data, determination of statistical significance, determination of statistical correlations, clustering algorithms, and the like.

A “sample” in the context of the present teachings refers to any biological sample that is isolated from a subject, generally a sample comprising cell free DNA. Samples for obtaining circulating cell-free DNA may include any suitable sample, often blood or blood-derived products, such as plasma, serum, etc. Alternative samples may include, for example, urine, ascites, synovial fluid, cerebrospinal fluid, saliva, and the like.

A “dataset” is a set of numerical values resulting from evaluation of a sample (or population of samples) under a desired condition. The values of the dataset can be obtained, for example, by experimentally obtaining measures from a sample and constructing a dataset from these measurements; or alternatively, by obtaining a dataset from a service provider such as a laboratory, or from a database or a server on which the dataset has been stored. Similarly, the term “obtaining a dataset associated with a sample” encompasses obtaining a set of data determined from at least one sample. Obtaining a dataset encompasses obtaining a sample, and processing the sample to experimentally determine the data, e.g., via measuring antibody binding, or other methods of quantitating a signaling response. The phrase also encompasses receiving a set of data, e.g., from a third party that has processed the sample to experimentally determine the dataset.

“Measuring” or “measurement” in the context of the present teachings refers to determining the presence, absence, quantity, amount, or effective amount of a substance in a clinical or subject-derived sample, including the presence, absence, or concentration levels of such substances, and/or evaluating the values or categorization of a subject's clinical parameters based on a control, e.g. baseline levels of the marker.

Classification can be made according to predictive modeling methods that set a threshold for determining the probability that a sample belongs to a given class. The probability preferably is at least 50%, or at least 60% or at least 70% or at least 80% or higher. Classifications also can be made by determining whether a comparison between an obtained dataset and a reference dataset yields a statistically significant difference. If so, then the sample from which the dataset was obtained is classified as not belonging to the reference dataset class. Conversely, if such a comparison is not statistically significantly different from the reference dataset, then the sample from which the dataset was obtained is classified as belonging to the reference dataset class.

The predictive ability of a model can be evaluated according to its ability to provide a quality metric, e.g. AUC or accuracy, of a particular value, or range of values. In some embodiments, a desired quality threshold is a predictive model that will classify a sample with an accuracy of at least about 0.7, at least about 0.75, at least about 0.8, at least about 0.85, at least about 0.9, at least about 0.95, or higher. As an alternative measure, a desired quality threshold can refer to a predictive model that will classify a sample with an AUC (area under the curve) of at least about 0.7, at least about 0.75, at least about 0.8, at least about 0.85, at least about 0.9, or higher.

As is known in the art, the relative sensitivity and specificity of a predictive model can be “tuned” to favor either the selectivity metric or the sensitivity metric, where the two metrics have an inverse relationship. The limits in a model as described above can be adjusted to provide a selected sensitivity or specificity level, depending on the particular requirements of the test being performed. One or both of sensitivity and specificity can be at least about at least about 0.7, at least about 0.75, at least about 0.8, at least about 0.85, at least about 0.9, or higher.

The term “antibody” includes full length antibodies and antibody fragments, and can refer to a natural antibody from any organism, an engineered antibody, or an antibody generated recombinantly for experimental, therapeutic, or other purposes as further defined below. Examples of antibody fragments, as are known in the art, such as Fab, Fab′, F(ab′)2, Fv, scFv, or other antigen-binding subsequences of antibodies, either produced by the modification of whole antibodies or those synthesized de novo using recombinant DNA technologies. The term “antibody” comprises monoclonal and polyclonal antibodies. Antibodies can be antagonists, agonists, neutralizing, inhibitory, or stimulatory. They can be humanized, glycosylated, bound to solid supports, and possess other variations.

The methods the invention may utilize affinity reagents comprising a label, labeling element, or tag. By label or labeling element is meant a molecule that can be directly (i.e., a primary label) or indirectly (i.e., a secondary label) detected; for example a label can be visualized and/or measured or otherwise identified so that its presence or absence can be known. Labels include optical labels such as fluorescent dyes or moieties. Fluorophores can be either “small molecule” fluors, or proteinaceous fluors (e.g. green fluorescent proteins and all variants thereof). In some embodiments, activation state-specific antibodies are labeled with quantum dots as disclosed by Chattopadhyay et al. (2006) Nat. Med. 12, 972-977. Quantum dot labeled antibodies can be used alone or they can be employed in conjunction with organic fluorochrome—conjugated antibodies to increase the total number of labels available. As the number of labeled antibodies increase so does the ability for subtyping known cell populations.

The detecting, sorting, or isolating step of the methods of the present invention can entail fluorescence-activated cell sorting (FACS) techniques or flow cytometry, mass cytometry, etc., where FACS is used to select cells from the population containing a particular surface marker, or the selection step can entail the use of magnetically responsive particles as retrievable supports for target cell capture and/or background removal. A variety of FACS systems are known in the art and can be used in the methods of the invention (see e.g., W099/54494, filed Apr. 16, 1999; U.S. Ser. No. 20010006787, filed Jul. 5, 2001, each expressly incorporated herein by reference).

Mass cytometry, or CyTOF (DVS Sciences), is a variation of flow cytometry in which antibodies are labeled with heavy metal ion tags rather than fluorochromes. Readout is by time-of-flight mass spectrometry. This allows for the combination of many more antibody specificities in a single samples, without significant spillover between channels. For example, see Bodenmiller at a. (2012) Nature Biotechnology 30:858-867.

Affinity reagents such as antibodies also find use in, for example, immunohistochemistry to determine expression of an immune checkpoint protein, such as CD274 (PD-L1), B7-1, B7-2, 4-1BB-L, GITRL, etc. Alternatively, expression can be determined by any convenient method known in the art, e.g. mRNA hybridization, flow cytometry, mass cytometry, etc. A sample for analysis may include, for example, a tumor biopsy sample, such as a needle biopsy sample.

The present invention incorporates information disclosed in other applications and texts. The following patent and other publications are hereby incorporated by reference in their entireties: Alberts et al., The Molecular Biology of the Cell, 4th Ed., Garland Science, 2002; Vogelstein and Kinzler, The Genetic Basis of Human Cancer, 2d Ed., McGraw Hill, 2002; Michael, Biochemical Pathways, John Wiley and Sons, 1999; Weinberg, The Biology of Cancer, 2007; Immunobiology, Janeway et al. 7th Ed., Garland, and Leroith and Bondy, Growth Factors and Cytokines in Health and Disease, A Multi Volume Treatise, Volumes 1A and IB, Growth Factors, 1996.

Unless otherwise apparent from the context, all elements, steps or features of the invention can be used in any combination with other elements, steps or features.

General methods in molecular and cellular biochemistry can be found in such standard textbooks as Molecular Cloning: A Laboratory Manual, 3rd Ed. (Sambrook et al., Harbor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998). Reagents, cloning vectors, and kits for genetic manipulation referred to in this disclosure are available from commercial vendors such as BioRad, Stratagene, Invitrogen, Sigma-Aldrich, and ClonTech.

The invention has been described in terms of particular embodiments found or proposed by the present inventor to comprise preferred modes for the practice of the invention. It will be appreciated by those of skill in the art that, in light of the present disclosure, numerous modifications and changes can be made in the particular embodiments exemplified without departing from the intended scope of the invention. Due to biological functional equivalency considerations, changes can be made in protein structure without affecting the biological action in kind or amount. All such modifications are intended to be included within the scope of the appended claims.

The subject methods are used for prognostic, diagnostic and therapeutic purposes. As used herein, the term “treating” is used to refer to both prevention of relapses, and treatment of pre-existing conditions. The treatment of ongoing cancer to achieve durable clinical benefit is of particular interest.

The term “promoter fragmentation entropy” (PFE) as used herein refers to the relative diversity in DNA fragments length at or near transcription start sites (TSS) following digestion. Promoter fragment entropy is calculated using a modified Shannon's entropy index as PFE(TSS):=Eₖ[Σ_{i:1-5}P*(e_{TSS}>(1+k)×eᵢ)] where Eₖ[.] denotes the expected value with respect to the excess parameter k, and P\hat{ }* is the probability with respect to the Dirichlet distribution Dir(α*). Here, we used a Gamma distribution for k˜Γ(s=0.5,r=1), where Γ is the Gamma distribution with shape s and rate r.

The term “nucleosome depleted region” (NDR) is used herein refers to promoter regions in DNA that are free from nucleosomes. The lack of nucleosomes is often indicative of genes that are actively being expressed. NDR depth refers to the depth of sequencing occurring within nucleosome depleted regions. To guard against variations in depth across the genome, including from GC-content variation or somatic copy number changes, depth was normalized within each window flanking each TSS as defined by the user in counts per million (CPM) space. This normalized measure was denoted as nucleosome depleted region score, NDR, for each TSS.

The term “sequencing depth” or “depth” refers to a total number of sequence reads or read segments at a given genomic location or loci from a test sample from an individual.

The term “selector” or “selector set” refers to an oligonucleotide or a set of oligonucleotides which correspond to specific genomic regions wherein genomic regions may comprise a TSS or a plurality of TSSs. A variety of selector and selector sets are known in the art (see e.g., US 2014-0296081 A1, filed Mar. 13, 2014 which has been expressly incorporated herein by reference).

### Methods of the Invention

Methods are provided for non-invasively determining the expression of genes of interest. The expression profile of these genes of interest are then used for numerous applications. These methods include, without limitation, methods for determining whether an individual with cancer will have a durable clinical benefit from treatment with an immune checkpoint inhibitor, methods for determining whether an individual with non-small cell lung carcinoma (NSCLC) is classified as adenocarcinomas (LUAD) or squamous cell carcinomas (LUSC), methods for quantifying tumor burden in individuals living with diffuse large B cell lymphoma (DLBCL), methods for determining the cell of origin in individuals living with DLBCL, etc. Provided is an integrated analytic method, where a a single biomarker is derived from promoter fragment entropy (PFE) and analysis of nucleosome depleted regions (NDR) depth, to generate a prognostic for patient responsiveness to immune checkpoint inhibition (ICI), a determination of NSCLC subtype, a determination of DLBCL tumor burden, and/or a DLBCL cell of origin classification. In some embodiments that use only noninvasive blood draws, the methods robustly identify which patients will achieve durable clinical benefit from immune checkpoint inhibition, what the cancer subtype classification is and/or what the tumor burden is. In an embodiment, the methods further comprise selecting a treatment regimen for the individual based on the analysis. In some embodiments, the prediction is based on samples shortly after a first ICI treatment.

A sample for cell free DNA profiling can be any suitable type that allows for the analysis of one or more DNA sample, preferably a blood sample. Samples can be obtained once or multiple times from an individual. Multiple samples can be obtained at different times from the individual. In some embodiments a sample is obtained prior to ICI treatment. In some embodiments a sample is obtain following a first ICI treatment, and within about 4 weeks, 3 weeks, 2 weeks, 1 week, of a first ICI treatment. In some embodiments a sample is obtained both prior to and following ICI treatment.

Samples of cell free DNA can be isolated from body samples. The cell free DNA can be separated from body samples by red cell lysis, centrifugation, elutriation, density gradient separation, apheresis, affinity selection, panning, FACS, centrifugation with Hypaque, solid supports (magnetic beads, beads in columns, or other surfaces) with attached antibodies, etc. The samples are analyzed as described above for the specific metric of interest.

The use of cfDNA in the determination of gene expression through inference provides advantages over RNA based methods of analyzing gene expression. The use of cfDNA provides a noninvasive means for the determination of gene expression through inference because obtaining cfDNA only requires a blood sample and does not require extensive tissue processing like RNA based methods require. cfDNA also provides the distinct advantage over RNA by being much more stable and less prone to degradation.

The methods of the invention include optimized library preparation methods with a multi-phase bioinformatics using a “selector” population of DNA oligonucleotides, which correspond to TSS regions in the genes of interest. The selector population of DNA oligonucleotides, which may be referred to as a selector set, comprises probes for a plurality of genomic regions.

In some embodiments of the invention, methods are provided for the identification of a selector set appropriate for a specific tumor type. Also provided are oligonucleotide compositions of selector sets, which may be provided adhered to a solid substrate, tagged for affinity selection, etc.; and kits containing such selector sets. Included, without limitation, is a selector set suitable for analysis of non-small cell lung carcinoma (NSCLC).

In other embodiments, methods are provided for the use of a selector set in the diagnosis and monitoring of cancer in an individual patient. In such embodiments the selector set is used to enrich, e.g. by hybrid selection, for cfDNA that corresponds to the TSS regions. The “selected” cfDNA is then amplified and sequenced.

Fully robotic or microfluidic systems include automated liquid-, particle-, cell- and organism-handling including high throughput pipetting to perform all steps of screening applications. This includes liquid, particle, cell, and organism manipulations such as aspiration, dispensing, mixing, diluting, washing, accurate volumetric transfers; retrieving, and discarding of pipet tips; and repetitive pipetting of identical volumes for multiple deliveries from a single sample aspiration. These manipulations are cross-contamination-free liquid, particle, cell, and organism transfers. This instrument performs automated replication of microplate samples to filters, membranes, and/or daughter plates, high-density transfers, full-plate serial dilutions, and high capacity operation.

In some embodiments, platforms for multi-well plates, multi-tubes, holders, cartridges, minitubes, deep-well plates, microfuge tubes, cryovials, square well plates, filters, chips, optic fibers, beads, and other solid-phase matrices or platform with various volumes are accommodated on an upgradable modular platform for additional capacity. This modular platform includes a variable speed orbital shaker, and multi-position work decks for source samples, sample and reagent dilution, assay plates, sample and reagent reservoirs, pipette tips, and an active wash station. In some embodiments, the methods of the invention include the use of a plate reader.

In some embodiments, interchangeable pipet heads (single or multi-channel) with single or multiple magnetic probes, affinity probes, or pipetters robotically manipulate the liquid, particles, cells, and organisms. Multi-well or multi-tube magnetic separators or platforms manipulate liquid, particles, cells, and organisms in single or multiple sample formats.

In some embodiments, the instrumentation will include a detector, which can be a wide variety of different detectors, depending on the labels and assay. In some embodiments, useful detectors include a microscope(s) with multiple channels of fluorescence; plate readers to provide fluorescent, ultraviolet and visible spectrophotometric detection with single and dual wavelength endpoint and kinetics capability, fluorescence resonance energy transfer (FRET), luminescence, quenching, two-photon excitation, and intensity redistribution; CCD cameras to capture and transform data and images into quantifiable formats; and a computer workstation.

In some embodiments, the robotic apparatus includes a central processing unit which communicates with a memory and a set of input/output devices (e.g., keyboard, mouse, monitor, printer, etc.) through a bus. Again, as outlined below, this can be in addition to or in place of the CPU for the multiplexing devices of the invention. The general interaction between a central processing unit, a memory, input/output devices, and a bus is known in the art. Thus, a variety of different procedures, depending on the experiments to be run, are stored in the CPU memory.

### Modeling and Statistical Methods

Mapping, deduplication and quality control of TSS sites and samples was preformed using FASTQ files that were demultiplexed using a custom pipeline wherein read pairs were considered only if both 8-bp sample barcodes and 6-bp UI Ds matched expected sequences after error-correction. After demultiplexing, barcodes were removed, and adaptor read-through was trimmed from the 3′ end of the reads using fastp to preserve short fragments. Fragments were aligned to human genome (hg19) using BWA; importantly, the disabled the automated distribution inference in BWA ALN was disabled to allow inclusion of shorter and longer cfDNA fragments that would otherwise be anomalously flagged as improperly paired. PCR duplicates were removed using a customized barcoding approach, which combines endogenous and exogenous unique molecular identifiers (UMIDs), including cfDNA fragment start and end positions, as well as pre-specified UMIDs within ligated adapters into account. To allow coverage uniformity for comparisons, data was down-sampled to a desired depth using ‘samtools view-s’. Desired depths include, without limitation, a depth of greater than 500×, a depth from 500 to 600×, from 600 to 700×, from 700 to 800×, from 800 to 900×, from 900 to 1000×, from 1000 to 1100×, from 1100 to 1200×, from 1200 to 1300×, from 1300 to 1400×, from 1400 to 1500×, from 1500 to 1600×, from 1600 to 1700×, from 1700 to 1800×, from 1800 to 1900×, from 1900 to 2000×, 2000 to 2100×, from 2100 to 2200×, from 2200 to 2300×, from 2300 to 2400×, from 2400 to 2500×, from 2500 to 2600×, from 2600 to 2700×, from 2700 to 2800×, from 2800 to 2900×, from 2900 to 3000×, or a sequencing depth of greater than 3000×. Samples with a sequencing depth of less than 500× were considered and any samples not meeting this depth threshold (median depth) were considered to fail quality control (QC). Any samples whose cfDNA fragment length density mode was below 140 or above 185 were also removed, since the expected fragment length density mode is 167 (corresponding to the chromatosomal DNA length). To identify and censor noisy sites among the 236 TSS regions profiled by our EPIC-Seq panel, 23 controls were profile, allowing the identification and removal stereotyped regions with reproducibly low TSS coverage (i.e., any site with CPM less than one third of uniformly distributed coverage across the TSSs in the selector, i.e.,

$$\frac{{10}^{6}}{236} \times \frac{1}{3} ,$$

in more than 75% of controls).

To guarantee adequate quality of fragments entering analysis, mapping quality was required (MAPQ, k) of>30 or>10 in the WGS and EPIC-Seq data, respectively (using ‘samtools view-q k-F3084’). The more lenient EPIC-seq MAPQ threshold was qualified by more stringent mappability and uniqueness requirements already imposed on the TSS regions selected during EPIC-seq selector design. The analysis was limited to reads with the following BAM FLAG set: 81, 93, 97, 99, 145, 147, 161, and 163. To ensure removal of non-unique fragments, reads with duplicate names were censored.

Fragmentomic feature extraction & summarization were conducted using 5 cfDNA fragmentomic features at TSS regions and then compared each of these features to gene expression, including Window Protection Score (WPS), Orientation-aware CfDNA Fragmentation (OCF), Motif Diversity Score (MDS), Nucleosome depleted region score (NDR), and Promoter Fragmentation Entropy (PFE). MDS, NDR, OCF, and WPS were each computed as per the conventions of the originally describing studies with minor modifications, as detailed below.

Motif diversity score (MDS) was determined as a performed end-motif sequence analysis of individual cfDNA fragments to assess the distribution of nucleotides among the first few positions for the reads of each read pair. This was performed by computationally extracting the first four 5′ nucleotides of the genomic reference sequence for each sequence read, resulting in a 4-mer sequence motif. MDS was then computed as the Shannon index of the distribution across 256 motifs (4-mers) at each TSS site, when considering fragments overlapping the 2 kb window flanking each TSS.

Nucleosome depleted region score (NDR) was calculated using the depth, normalized within each window flanking each TSS in counts per million (CPM) space. This normalized measure was denoted as the nucleosome depleted region score, NDR, for each TSS.

Promoter fragmentation entropy (PFE) was calculated using Shannon entropy to summarize the diversity in cfDNA fragment size values in the vicinity of each TSS site as defined by the user. 201 size-bins were defined [from b₁=100 bps to b₂₀₁=300 bps] and estimated the density by the maximum-likelihood, i.e., \hat{p}=[p₁, . . . , p₂₀₁] with

$$= \frac{{n}_{i}}{n}$$

where nᵢ and n denote the number of fragments with length bᵢ and total number of fragments at the TSS, respectively. Shannon's entropy was calculated as − [symbol] log2 - [symbol] and then normalized as follows. To account for variations in sequencing depth from sample to sample as well as other hidden factors impacting overall cfDNA fragment length distributions that might confound PFE, we defined a relative entropy using a Bayesian approach through a Dirichlet-multinomial model. In this model, fragment size profiles in a given cfDNA sample are assumed to follow a multinomial distribution (p) whose probability mass function is itself governed by a Dirichlet distribution, p˜Dirichlet(α), where vector α represents the parameter vector of the Dirichlet distribution. Here, we first used a set of genes to create a background fragment length density as α. For the background distribution, two flanking regions were focused on, (a) −1 Kbps (upstream) to −750 bps (upstream) and (b) from +750 bps (downstream) to +1 Kbps (downstream). The fragments that fell within those regions were used for the background fragment length distributions. Five background gene subsets were randomly selected and calculated their Shannon entropies, denoting these by e₁, e₂, e₃, e₄, and e₅. For a given TSS, the posterior of the Dirichlet distribution was calculated, i.e., Dir(α*=α+[\hat{n}₁, . . . , \hat{n}₂₀₁]). The Shannon entropy of a given TSS was then compared with the five randomly generated entropies to measure the excess in diversity in the fragment length values at the TSS of interest. Formally, PFE was defined as PFE(TSS):=Eₖ[Σ_{i:1-5}P*(e_{TSS}>(1+k)×eᵢ)] where Eₖ[.] denotes the expected value with respect to the excess parameter k, and P* is the probability with respect to the Dirichlet distribution Dir(α*). Here, we used a Gamma distribution for k˜Γ(s=0.5,r=1), where Γ is the Gamma distribution with shape s and rate r.

Whole exome PFE analysis was performed using the raw Shannon entropy (as described in ‘Fragment length diversity calculation using Shannon entropy’) at any given gene, after transforming it into a z-score, using a cohort of 34 cfDNA WES profiles (each with 200-400× depth). To account for differences in depth in the cohort for normalization, meta-profiles of 5 samples were considered to achieve comparable depths as those initially used to relate PFE and gene expression levels when relying on WGS.

Small cell lung cancer gene signature set was generated using an RNA-Seq data of 81 SCLC primary tumors. Differential gene expression analysis was performed by comparing the RNA-seq data of these tumors with our reference PBMC RNA expression levels and identified genes in the top 1500 of SCLC expression overlapping genes in the bottom 5000 of the PBMC expression (‘high in SCLC’). Similarly, for ‘low in SCLC’ genes, we selected genes which are in top 1500 of PBMC expression and bottom 5,000 of SCLC expression. The gene set was further limited to those whose TSSs were covered in our whole exome panel to ensure sufficient sequencing coverage for analysis.

To infer RNA expression levels from cfDNA fragmentation profiles at TSS regions of genes across the transcriptome, a prediction model was built using two features, PFE and NDR. Of note, among the 5 fragmentomic features considered, these indices demonstrate highest individual correlations as well as complementarity. For training, one cfDNA sample sequenced to high coverage depth by WGS was employed. RNA-Seq was performed on the PBMC of five healthy subjects and used the average across three of these individuals as the ‘reference expression vector’. Next, to achieve a higher resolution at the core promoters, every 10 genes was grouped, based on their expression in our reference RNA-seq vector. After removing genes used as background for calculating PFE, a total of 1,748 groups (of 10 genes each) remained. All the fragments at the extended core promoters were pooled of the genes within each group and extracted the two features: NDR and PFE. The two features were normalized by 95% quantile over the background genes, where for PFE the normalization is

$$= min ( 1 , \frac{PFE}{Γ ( \frac{Q ( \{ PFE \} , 95 )}{{PFE}_{Bg}} ; 0.5 , 1 )} ) and = \frac{NDR}{{NDR}_{Bg}} ,$$

where Q(.,k) denotes the k^{th} quantile. By bootstrap resampling, we then built 600 ensemble models: 200 univariable PFE-alone-models m_{PFE,1}, m_{PFE,2}, . . . , m_{PFE,200}, 200 univariable NDR-alone-models m_{NDR,1}, m_{NDR,2}, . . . , m_{NDR,200} and 200 NDR-PFE integrated models m_{Int,1}, m_{Int,2}, . . . , m_{Int,200}.

To transfer this expression prediction model—which was originally derived from WGS—to the targeted TSS space (EPIC-seq), each of the 600 models above were evaluated, by measuring its root mean squared error (RMSE) on two held out healthy subjects. For each of these two healthy subjects, the cfDNA profile was compared by EPIC-seq to the corresponding PBMC transcriptome profile by RNA-Seq from the same blood specimen and computed the RMSE for each of the 600 ensemble models. The weight of each model was then proportionally scaled by the inverse RMSE of that model, with the final score then calculated as the linear sum of 600 models, weighted as described above.

Identification of cancer type-specific genes was conducted using the TCGA and DLBCL gene expression data sets in the form of RNA-Seq FPKM-UQ for all individuals using the GDC API. After removing samples from individuals with a history of more than one type of malignancy, were divided into two separate cohorts for training and validation (70% and 30% of each cancer type respectively). In the training set for each cancer type, median gene expression (FPKM-UQ) was calculated and protein coding genes in the upper 15th quantile were considered as highly expressed genes. To remove potentially confounding effects in cfDNA from variation in blood cells, genes within the upper 5^{th} quantile of expression in peripheral blood were excluded, when considering whole-blood transcriptome profiles from GTEx.

Gene selection for EPIC-Seq targeted sequencing panel design was determined with known molecular subtypes exhibiting distinct gene expression profiles. Cancer-specific genes for LUAD, LUSC, and DLBCL were included. To find subtype-specific genes in NSCLC, differential expression analysis was performed using the DESeq2 package in R Bioconductor to distinguish LUAD and LUSC tumor transcriptomes from the TCGA. For the lymphoma analysis, a list of genes previously shown as differentially expressed between ABC and GCB subtypes according to RNA-Seq gene expression data was used. In addition to these DLBCL and NSCLC specific genes, 50 genes from the LM22 gene set were included capturing variation in peripheral blood leukocyte counts. Together these and other control genes contributed to a total of 179 unique genes, with each gene contributing one or more TSS regions to EPIC-Seq totaling 236 targeted TSS regions.

Distinguishing lung cancer (EPIC-Lung classifier) was trained to distinguish lung cancer from non-cancer subjects. All the TSSs for immune cell type and NSCLC histology classification were used in this classifier. For genes with multiple TSS regions, in each iteration of cross-validation, TSS regions were first combined with intra-gene correlation exceeding 0.95 and capturing the mean. For those with correlation less than 0.95, individual TSS regions were preserved as independent reporters. This resulted in 139 features in the model and 143 samples (67 lung cancer cases and 71 controls). An [symbol] ₁- [symbol] ₂—regularized logistic regression model was trained (‘elastic net’ with α=0.9) and an optimal λ obtained by cross-validation. The full model was evaluated through a leave-one-batch out (LOBO) model. Here, every batch contained at least one sample, and representing a set of samples that were either captured and/or sequenced together in one NGS sequencing lane.

A NSCLC histology subtype classifier was designed to distinguish the two major subtypes of non-small cell lung cancer, i.e., lung adenocarcinoma (LUAD) and lung squamous cell carcinoma (LUSC). Similar to the model in ‘EPIC-Lung classifier’, the classification model employs elastic net with α=0.9, with multiple TSS sites corresponding to one gene being merged. The performance of this classifier was evaluated via leave-one-out (LOO) analysis. The classifier was trained using 80 features with 67 samples (36 LUADs and 31 LUSCs). To evaluate performance, classification accuracy with equal weights was calculated.

The significance of the model coefficients in the NSCLC histology classifier from plasma cfDNA using EPIC-Seq was assessed and their concordance with prior design from tumor transcriptomes using RNA-Seq. Specifically, nonzero coefficients were compared from the elastic net model from cfDNA profiling, and then performed a t-test for the LUAD genes coefficients vs LUSC genes coefficients.

To predict benefit from immune checkpoint inhibitors, the differentially expressed TSSs in a discovery pre-treatment cohort was indentified (non-ICI; lung cancer vs normal). The following TSS regions from genes with Bonferroni-corrected P<0.25 with a 1-sided t-test were nominated: (FOLR1 TSS#3, ITGA3 TSS#1, LRRC31 TSS#1, MACC1 TSS#1, NKX2-1 TSS#2, SCNN1A TSS#2, SFTPB TSS#1, WFDC2 TSS#1, CLDN1 TSS#1, FSCN1 TSS#1, GPC1 TSS#1, KRT17 TSS#1, PFN2 TSS#1, PKP1 TSS#1, S100A2 TSS#1, SFN TSS#1, SOX2 TSS#2, TP63 TSS#2). Denoting the expression levels of these genes by ξₜ_{₀}=(x₁^{t}^{₀}, . . . , xₖ^{t}^{₀}) and ξₜ_{₁}=(x₁^{t}^{₁}, . . . , xₖ^{t}^{₁}) for time point t₀ and t₁, respectively, (fold-change) statistics were defined as

$$s ( {ξ}_{{t}_{0}} , {ξ}_{{t}_{1}} ) = log \frac{\overline{{ξ}_{{t}_{1}}}}{\overline{{ξ}_{{t}_{0}}}}$$

where (̅.̅)̅ is used to denote averaging the vector elements. For each patient, empirical derivation of a null distribution for the s statistics by randomly selecting k sites from the EPIC-Seq selector. An empirical left-sided P-value was then calculated to measure response to therapy. The EPIC-seq dynamics score was then defined as the logarithm (base 10) of these empirical P-values.

A classifier was trained to distinguish DLBCL from non-cancer subjects using elastic-net, with regularization parameters being set as in ‘EPIC-Lung classifier’. The dataset used for LOBO cross-validation comprised 129 features and 167 samples (91 DLBCL cases and 71 controls).

For the classification of DLBCL COO, a GCB score was defined as follows: (1) within a leave-one-out cross-validation framework, each gene expression was standardized (i.e. the Z-score) and converted the Z-scores into probabilities, and then (2) defined a COO score as

$$- log 10 ( \frac{\sqrt[1 / \vert GCB \vert]{{\prod }_{i ∈ GCB} {p}_{i}}}{\sqrt[1 / \vert ABC \vert]{{\prod }_{i ∈ ABC} {p}_{i}}} ) .$$

Gene sets for each subtype were defined as originally selected in the EPIC-Seq selector design for DLBCL classification. To evaluate performance, the concordance was measured between EPIC-Seq scores and (1) genetic COO classification scores obtained from CAPP-Seq, as well as (2) labels from Hans immunohistochemical algorithm.

Associations between known and predicted variables were measured by Pearson correlation (r) or Spearman correlation (ρ) depending on data type. When data were normally distributed, group comparisons were determined using t-test with unequal variance or a paired t-test, as appropriate; otherwise, a two-sided Wilcoxon test was applied. To test for trend in continuous variables vs categorical groups, Jonckheere's trend test was used as implemented in the clinfun R package. Correction for multiple hypothesis testing was performed using the Bonferroni method. Results with two-sided P<0.05 were considered significant. Statistical analyses were performed with R 4.0.1. Confidence intervals (CI) are calculated by re-sampling with replacement (i.e., bootstrapping). Receiver operating characteristic (ROC) curve analyses were performed using the R package pROC. Survival analyses were performed using R package survival. When dichotomized, Kaplan-Meier estimates were used to plot the survival curves and statistical significance was evaluated by log-rank test. Otherwise, Cox proportional-hazards models were fitted to the data to determine the significance of each co-variate.

In some embodiments, the invention provides kits for the classification, diagnosis, prognosis, theranosis, and/or prediction of an outcome. The kit may further comprise a software package for data analysis of the cellular state and its physiological status, which may include reference profiles for comparison with the test profile and comparisons to other analyses as referred to above. The kit may also include instructions for use for any of the above applications.

Kits provided by the invention may comprise one or more of the affinity reagents described herein, reagents for isolation and sequencing analysis of cfDNA, etc. A kit may also include other reagents that are useful in the invention, such as modulators, fixatives, containers, plates, buffers, therapeutic agents, instructions, and the like.

Kits provided by the invention can comprise one or more labeling elements. Non-limiting examples of labeling elements include small molecule fluorophores, proteinaceous fluorophores, radioisotopes, enzymes, antibodies, chemiluminescent molecules, biotin, streptavidin, digoxigenin, chromogenic dyes, luminescent dyes, phosphorous dyes, luciferase, magnetic particles, beta-galactosidase, amino groups, carboxy groups, maleimide groups, oxo groups and thiol groups, quantum dots , chelated or caged lanthanides, isotope tags, radiodense tags, electron-dense tags, radioactive isotopes, paramagnetic particles, agarose particles, mass tags, e-tags, nanoparticles, and vesicle tags.

In some embodiments, the kits of the invention enable the detection of signaling proteins by sensitive cellular assay methods, such as IHC and flow cytometry, which are suitable for the clinical detection, classification, diagnosis, prognosis, theranosis, and outcome prediction.

Such kits may additionally comprise one or more therapeutic agents. The kit may further comprise a software package for data analysis of the physiological status, which may include reference profiles for comparison with the test profile.

Such kits may also include information, such as scientific literature references, package insert materials, clinical trial results, and/or summaries of these and the like, which indicate or establish the activities and/or advantages of the composition, and/or which describe dosing, administration, side effects, drug interactions, or other information useful to the health care provider. Such information may be based on the results of various studies, for example, studies using experimental animals involving in vivo models and studies based on human clinical trials. Kits described herein can be provided, marketed and/or promoted to health providers, including physicians, nurses, pharmacists, formulary officials, and the like. Kits may also, in some embodiments, be marketed directly to the consumer.

### Reports

In some embodiments, providing an evaluation of a subject for a classification, diagnosis, prognosis, theranosis, and/or prediction of an outcome includes generating a written report that includes the artisan's assessment of the subject's state of health i.e. a “diagnosis assessment”, of the subject's prognosis, i.e. a “prognosis assessment”, and/or of possible treatment regimens, i.e. a “treatment assessment”. Thus, a subject method may further include a step of generating or outputting a report providing the results of a diagnosis assessment, a prognosis assessment, or treatment assessment, which report can be provided in the form of an electronic medium (e.g., an electronic display on a computer monitor), or in the form of a tangible medium (e.g., a report printed on paper or other tangible medium).

A “report,” as described herein, is an electronic or tangible document which includes report elements that provide information of interest relating to a diagnosis assessment, a prognosis assessment, and/or a treatment assessment and its results. A subject report can be completely or partially electronically generated. A subject report includes at least a diagnosis assessment, i.e. a diagnosis as to whether a subject will have a particular clinical response, and/or a suggested course of treatment to be followed. A subject report can further include one or more of: 1) information regarding the testing facility; 2) service provider information; 3) subject data; 4) sample data; 5) an assessment report, which can include various information including: a) test data, where test data can include an analysis of cellular signaling responses to activation, b) reference values employed, if any.

The report may include information about the testing facility, which information is relevant to the hospital, clinic, or laboratory in which sample gathering and/or data generation was conducted. This information can include one or more details relating to, for example, the name and location of the testing facility, the identity of the lab technician who conducted the assay and/or who entered the input data, the date and time the assay was conducted and/or analyzed, the location where the sample and/or result data is stored, the lot number of the reagents (e.g., kit, etc.) used in the assay, and the like. Report fields with this information can generally be populated using information provided by the user.

The report may include information about the service provider, which may be located outside the healthcare facility at which the user is located, or within the healthcare facility. Examples of such information can include the name and location of the service provider, the name of the reviewer, and where necessary or desired the name of the individual who conducted sample gathering and/or data generation. Report fields with this information can generally be populated using data entered by the user, which can be selected from among pre-scripted selections (e.g., using a drop-down menu). Other service provider information in the report can include contact information for technical information about the result and/or about the interpretive report.

The report may include a subject data section, including subject medical history as well as administrative subject data (that is, data that are not essential to the diagnosis, prognosis, or treatment assessment) such as information to identify the subject (e.g., name, subject date of birth (DOB), gender, mailing and/or residence address, medical record number (MRN), room and/or bed number in a healthcare facility), insurance information, and the like), the name of the subject's physician or other health professional who ordered the susceptibility prediction and, if different from the ordering physician, the name of a staff physician who is responsible for the subject's care (e.g., primary care physician).

The report may include a sample data section, which may provide information about the biological sample analyzed, such as the source of biological sample obtained from the subject (e.g. blood, type of tissue, etc.), how the sample was handled (e.g. storage temperature, preparatory protocols) and the date and time collected. Report fields with this information can generally be populated using data entered by the user, some of which may be provided as pre-scripted selections (e.g., using a drop-down menu).

The report may include an assessment report section, which may include information generated after processing of the data as described herein. The interpretive report can include a prognosis of the likelihood that the patient will develop tumor benefit from immune checkpoint inhibitors. The interpretive report can include, for example, results of the analysis, methods used to calculate the analysis, and interpretation, i.e. prognosis. The assessment portion of the report can optionally also include a Recommendation(s). For example, where the results indicate the subject's prognosis for propensity to develop tumor benefit from immune checkpoint inhibitors.

It will also be readily appreciated that the reports can include additional elements or modified elements. For example, where electronic, the report can contain hyperlinks which point to internal or external databases which provide more detailed information about selected elements of the report. For example, the patient data element of the report can include a hyperlink to an electronic patient record, or a site for accessing such a patient record, which patient record is maintained in a confidential database. This latter embodiment may be of interest in an in-hospital system or in-clinic setting. When in electronic format, the report is recorded on a suitable physical medium, such as a computer readable medium, e.g., in a computer memory, zip drive, CD, DVD, etc.

It will be readily appreciated that the report can include all or some of the elements above, with the proviso that the report generally includes at least the elements sufficient to provide the analysis requested by the user (e.g., a diagnosis, a prognosis, or a prediction of responsiveness to a therapy).

### Computer Aspects

A computational system (e.g., a computer) may be used in the methods of the present disclosure to integrate and to analyze data generated from promoter fragment entropy and normalized NDR depth. A computational unit may include any suitable components to analyze the measured images. Thus, the computational unit may include one or more of the following: a processor; a non-transient, computer-readable memory, such as a computer-readable medium; an input device, such as a keyboard, mouse, touchscreen, etc.; an output device, such as a monitor, screen, speaker, etc.; a network interface, such as a wired or wireless network interface; and the like.

The raw data from measurements, such as promoter fragment entropy normalized NDR depth and the like, can be analyzed and stored on a computer-based system. As used herein, “a computer-based system” refers to the hardware means, software means, and data storage means used to analyze the information of the present invention. The minimum hardware of the computer-based systems of the present invention comprises a central processing unit (CPU), input means, output means, and data storage means. A skilled artisan can readily appreciate that any one of the currently available computer-based system are suitable for use in the present invention. The data storage means may comprise any manufacture comprising a recording of the present information as described above, or a memory access means that can access such a manufacture.

The analysis may be implemented in hardware or software, or a combination of both. In one embodiment of the invention, a machine-readable storage medium is provided, the medium comprising a data storage material encoded with machine readable data which, when using a machine programmed with instructions for using said data, is capable of displaying a any of the datasets and data comparisons of this invention. Such data may be used for a variety of purposes, such as diagnosis, disease treatment and the like. In some embodiments, the invention is implemented in computer programs executing on programmable computers, comprising a processor, a data storage system (including volatile and non-volatile memory and/or storage elements), at least one input device, and at least one output device. Program code is applied to input data to perform the functions described above and generate output information. The output information is applied to one or more output devices, in known fashion. The computer may be, for example, a personal computer, microcomputer, or workstation of conventional design.

Each program is preferably implemented in a high level procedural or object oriented programming language to communicate with a computer system. However, the programs can be implemented in assembly or machine language, if desired. In any case, the language can be a compiled or interpreted language. Each such computer program is preferably stored on a storage media or device (e.g., ROM or magnetic diskette) readable by a general or special purpose programmable computer, for configuring and operating the computer when the storage media or device is read by the computer to perform the procedures described herein. The system can also be considered to be implemented as a computer-readable storage medium, configured with a computer program, where the storage medium so configured causes a computer to operate in a specific and predefined manner to perform the functions described herein.

A variety of structural formats for the input and output means can be used to input and output the information in the computer-based systems of the present invention. One format for an output means test datasets possessing varying degrees of similarity to a trusted profile. Such presentation provides a skilled artisan with a ranking of similarities and identifies the degree of similarity contained in the test pattern.

The data and analysis thereof can be provided in a variety of media to facilitate their use. “Media” refers to a manufacture that contains the signature pattern information of the present invention. The databases of the present invention can be recorded on computer readable media, e.g. any medium that can be read and accessed directly by a computer. Such media include, but are not limited to: magnetic storage media, such as floppy discs, hard disc storage medium, and magnetic tape; optical storage media such as CD-ROM; electrical storage media such as RAM and ROM; and hybrids of these categories such as magnetic/optical storage media. One of skill in the art can readily appreciate how any of the presently known computer readable mediums can be used to create a manufacture comprising a recording of the present database information. “Recorded” refers to a process for storing information on computer readable medium, using any such methods as known in the art. Any convenient data storage structure can be chosen, based on the means used to access the stored information. A variety of data processor programs and formats can be used for storage, e.g. word processing text file, database format, etc.

A variety of structural formats for the input and output means can be used to input and output the information in the computer-based systems. Such presentation provides a skilled artisan with a ranking of similarities and identifies the degree of similarity contained in the test data.

Further provided herein is a method of storing and/or transmitting, via computer, sequence, and other, data collected by the methods disclosed herein. Any computer or computer accessory including, but not limited to software and storage devices, can be utilized to practice the present invention. Sequence or other data (e.g., immune repertoire analysis results), can be input into a computer by a user either directly or indirectly. Additionally, any of the devices which can be used to sequence DNA or analyze DNA or analyze immune repertoire data can be linked to a computer, such that the data is transferred to a computer and/or computer-compatible storage device. Data can be stored on a computer or suitable storage device (e.g., CD). Data can also be sent from a computer to another computer or data collection point via methods well known in the art (e.g., the internet, ground mail, air mail). Thus, data collected by the methods described herein can be collected at any point or geographical location and sent to any other geographical location.

### EXPERIMENTAL

The following examples are given for the purpose of illustrating various embodiments of the invention and are not meant to limit the present invention in any fashion. The present examples, along with the methods described herein are presently representative of preferred embodiments, are exemplary, and are not intended as limitations on the scope of the invention. Changes therein and other uses which are encompassed within the spirit of the invention as defined by the scope of the claims will occur to those skilled in the art.

### Example 1

In this study, we introduce EPIC-Seq, a novel approach that leverages cell-free DNA fragmentation patterns to allow non-invasive inference of gene expression, which can be used for a wide variety of clinically relevant applications including tumor detection, subtype classification, response assessment, and analysis of genes with prognostic implications. Compared to EPIC-Seq, the sensitivity of previously described cfDNA fragmentomic techniques and features has been insufficient to resolve expression of individual genes with high fidelity. The approach described here achieves substantially improved performance by leveraging the use of a new entropy-based fragmentomic metric (PFE), as well as higher sequencing depth achieved through targeted capture of promoter regions of genes of interest.

To allow inference of RNA expression levels from cfDNA fragmentomic features by EPIC-Seq, we focused our efforts on capturing features of cfDNA at transcription sites that reflect epigenetically encoded signals from nucleosomal accessibility and positioning, since these are key factors for determining transcriptional output. These fragmentomic signals appeared strongest at promoters of actively expressed genes when profiling cfDNA by whole genome sequencing motivating our TSS capture approach. However, we also observed significant signal at exonic regions of actively expressed genes in whole exome sequencing, suggesting opportunities to more broadly extend EPIC-Seq to study expression of genes of interest. In addition, tissue- and lineage-specificity are also provided by several other epigenetic signals that can be measured noninvasively, including 5mCpG and 5hmCpG modifications and specific histone posttranslational modifications.

As demonstrated below, EPIC-Seq is useful for a wide variety of clinically relevant cancer classification problems. Importantly, we demonstrate the utility of the inferred gene expression levels from EPIC-Seq using multiple independent lines of evidence. Specifically, we describe significant correlations of EPIC-Seq signals not only with expectations from tissue transcriptomic profiling, but also with disease burden as measured by total metabolic tumor volume and mutation-based ctDNA analysis. Furthermore, we observed significant correlation of EPIC-Seq signals with therapeutic responses to immunotherapy and chemotherapy, as well as its ability to assess expression of prognostically informative genes.

We focused on the noninvasive histological classification of lung cancers and the molecular classification of aggressive B-cell lymphomas, two common and representative cancer types where such classification is clinically routine but at times fraught by diagnostic challenges. The robust performance that we observed for the accurate classification of each of these tumor subtypes demonstrates that this approach can be broadly extended to other cancer types and other pathologies. For example, despite the many diagnostic tools already available in the United States, carcinomas of unknown primary (CUP) continue to represent some 2-5% of incident cancers. EPIC-Seq provides means for the classification of such carcinomas using non-invasive methods. Separately, the methods we describe have applications beyond cancer for the noninvasive detection of signals from cell types, tissues, and pathways and pathologies of interest. These include noninvasive strategies to detect tissue injury and ischemia, as well as pharmacodynamic effects on specific therapeutically targeted pathways and toxicity profiles for diverse human tissues that are otherwise difficult to monitor noninvasively (e.g., the brain and gastrointestinal tract), before symptomatic tissue damage occurs.

### Results

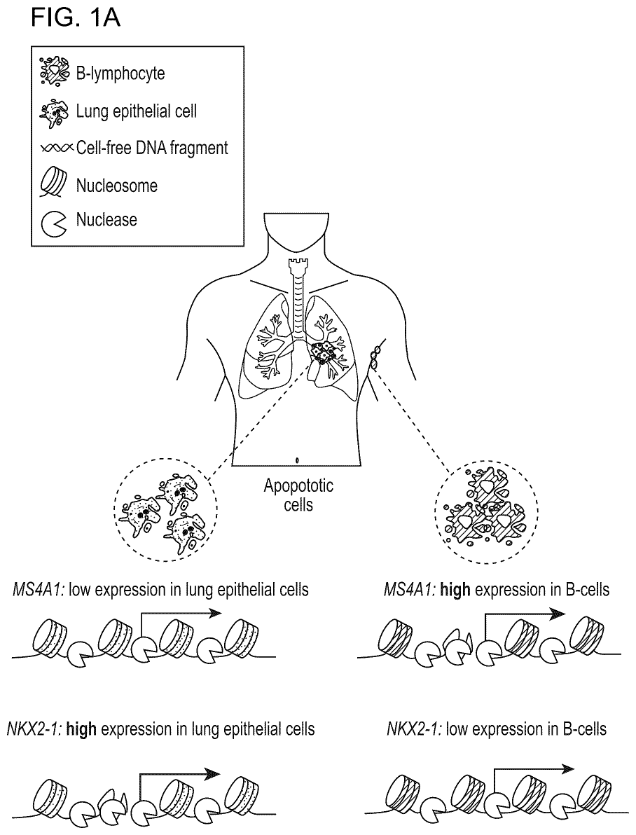

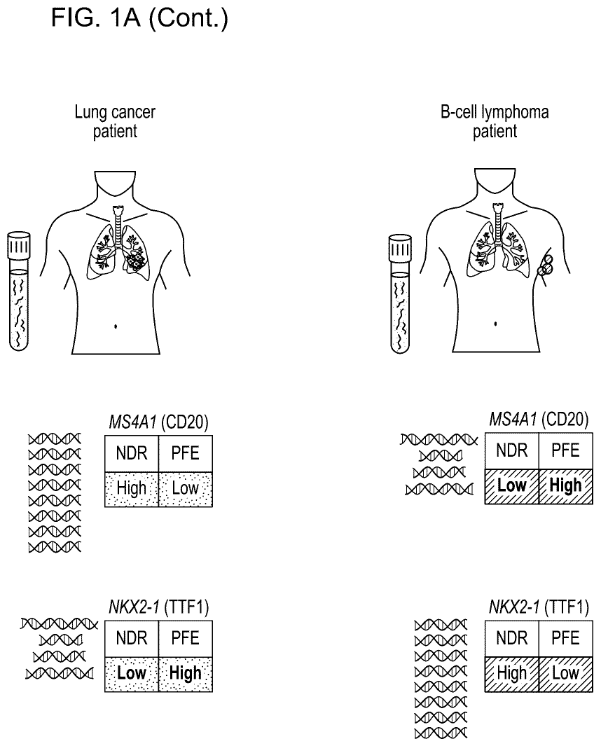

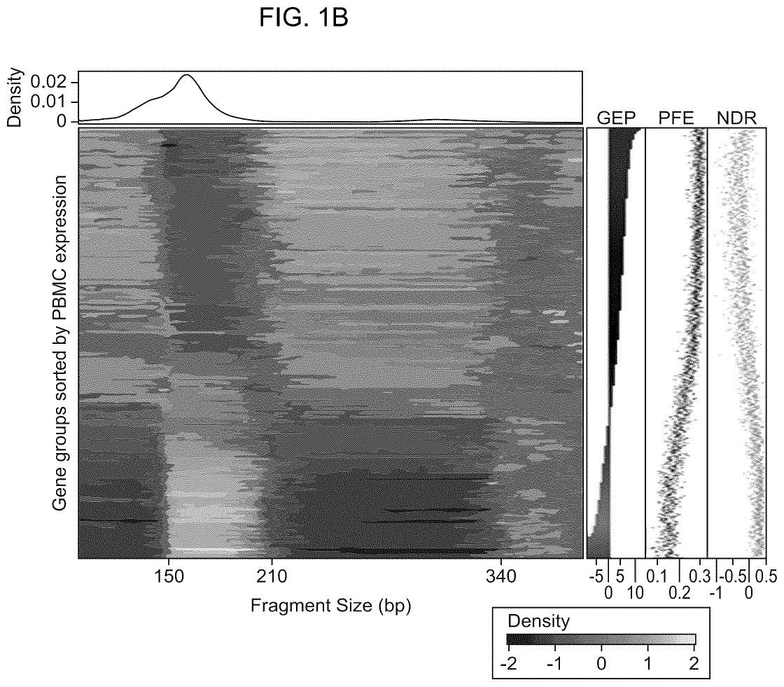

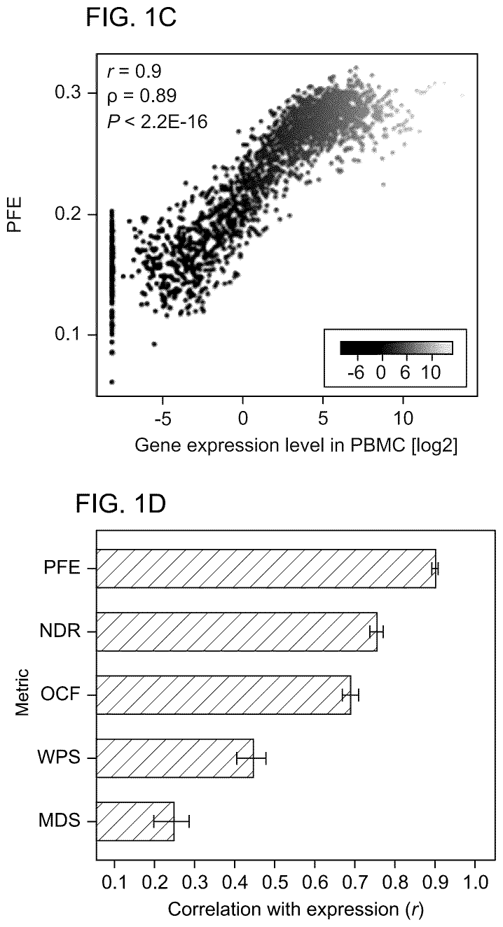

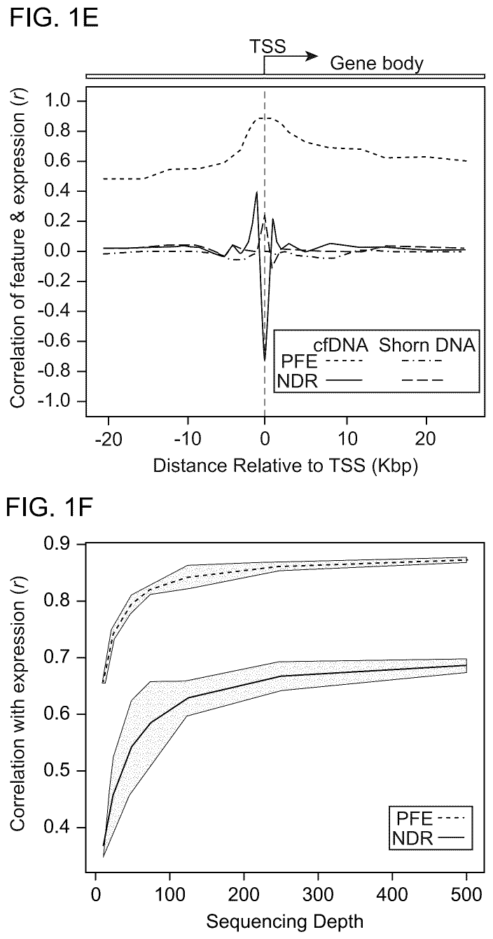

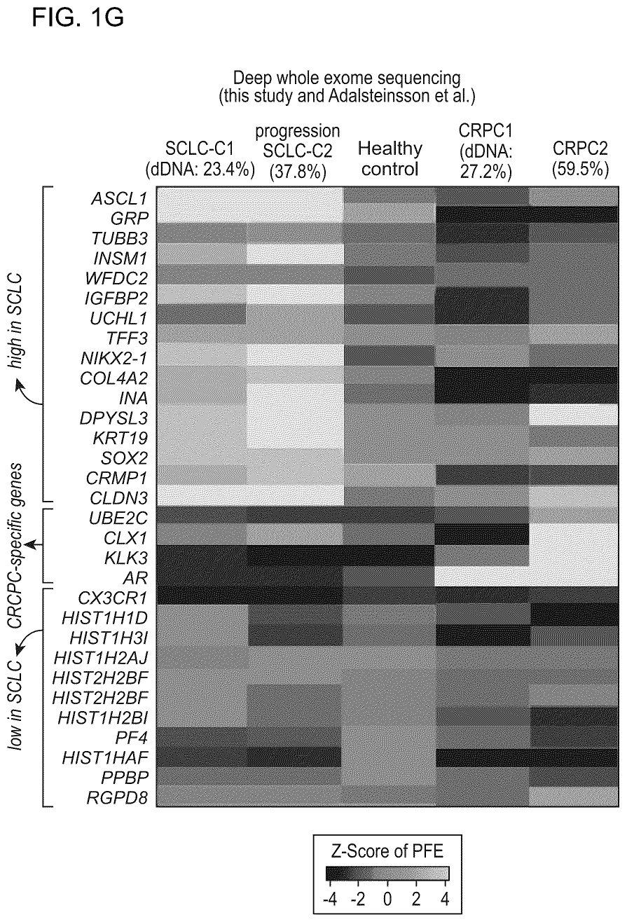

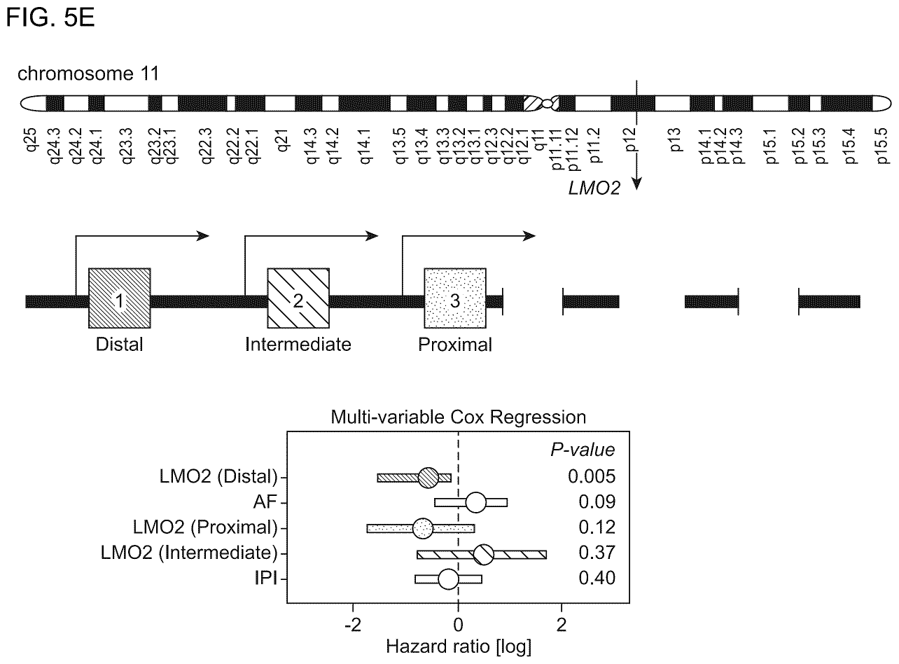

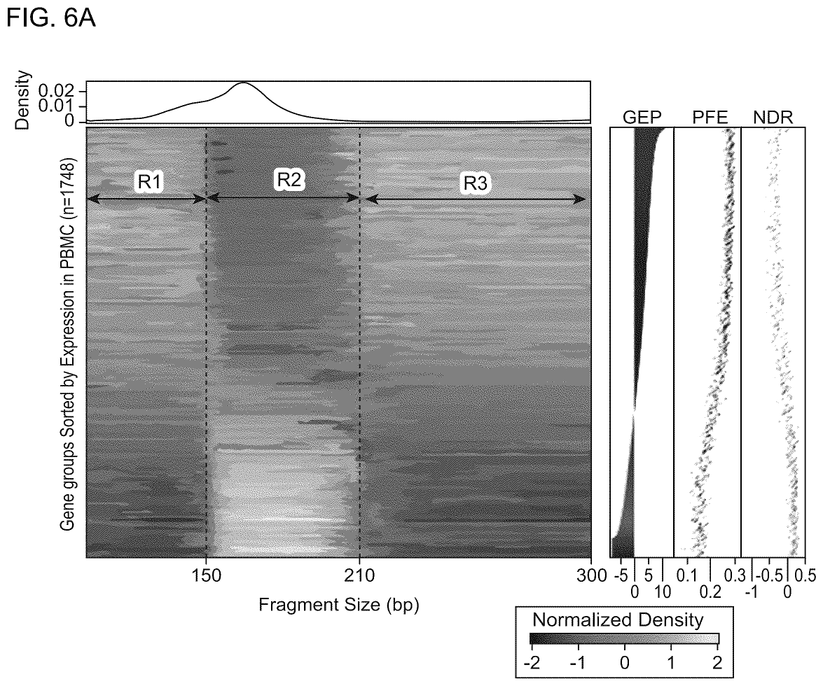

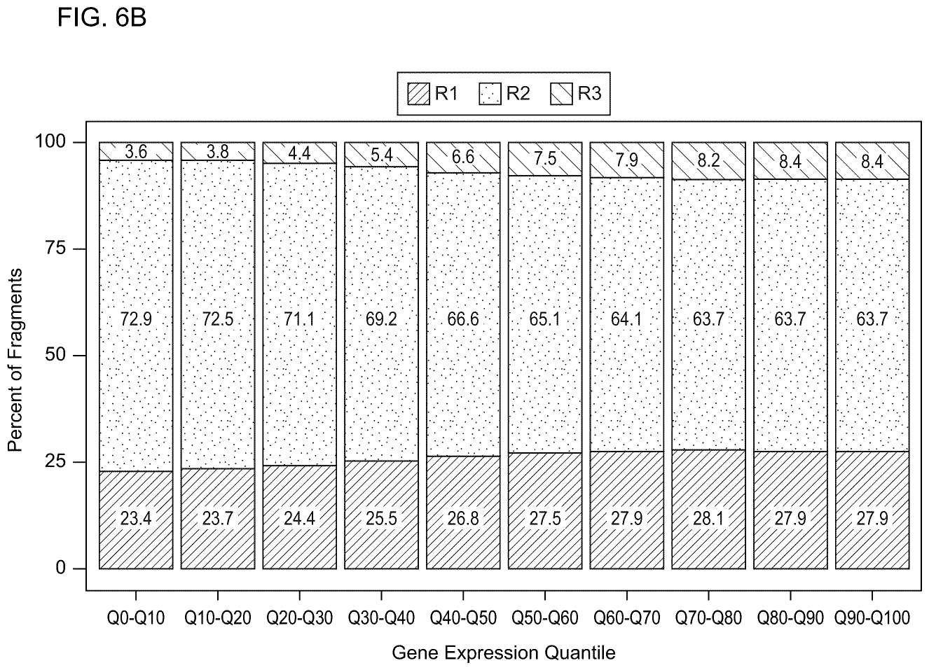

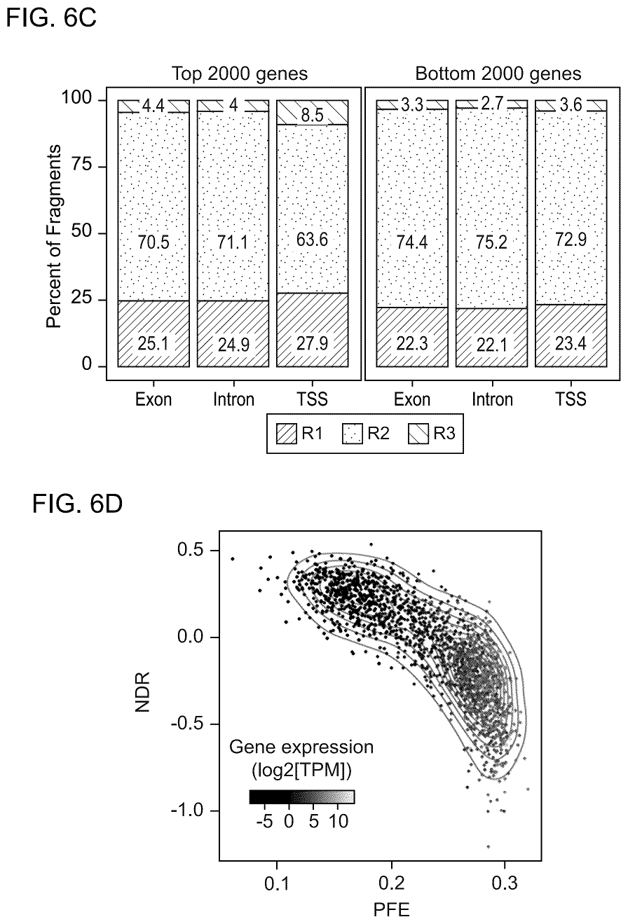

Cell-free DNA features correlated with gene expression. We hypothesized that cfDNA fragments from active promoters (which are less protected by nucleosomes) will exhibit more random cleavage patterns than fragments from inactive promoters (which are more protected by nucleosomes). If correct, this allows inferences about the expression of individual genes from cfDNA (FIG. 1a). To explore this hypothesis, we profiled cfDNA by relatively deep WGS (250×) from a patient with carcinoma of unknown primary (CUP) but very low levels of ctDNA as quantified by personalized CAPP-Seq (<0.05%; Methods). Since the vast majority of cfDNA molecules were therefore of hematopoietic origin, we correlated specific cfDNA fragmentomic features to expression levels of peripheral blood leukocytes determined by RNA-Seq. We then ranked genes by their expression levels and characterized the distribution of cfDNA fragments at their promoters (FIG. 1b). In support of our hypothesis, cfDNA molecules mapping to the ˜2 kb region flanking the TSSs of highly expressed genes exhibit substantially more fragment length diversity than fragments mapping to TSSs of poorly expressed genes. This phenomenon is especially prominent in subnucleosomal fragments (<150 bp and 210-300 bp, FIG. 1b and FIGS. 6a-b).

We reasoned that nucleosome displacement or depletion at the TSS of active genes could result in more diverse digested fragments, and that estimating this diversity could inform the corresponding expression level at individual gene TSS regions. We therefore captured this diversity in cfDNA fragment lengths as an entropy measure, calculating a modified Shannon's index for fragment lengths at each gene's TSS, a normalized metric that we call promoter fragmentation entropy (PFE; Methods). We observed remarkably high transcriptome-wide correlation between PFE measured in cfDNA by WGS and expression levels measured by RNA-Seq of peripheral blood mononuclear cells (PBMCs; R=0.89, P<1E−16; FIG. 1b-c). While sequencing depth at the nucleosome-depleted regions flanking the TSS (NDR depth) was also significantly correlated with gene expression of corresponding genes, it showed substantially lower correlation than did PFE (FIG. 1b; r=−0.78, P<1E−16). The significant correlations between RNA expression levels and fragmentomic features were only observed in cfDNA and not in acoustically shorn high-molecular-weight genomic DNA from matched leukocytes (PFE r=0.003; NDR r=0.24). Accordingly, the expression inferences from cfDNA fragmentation profiles appear to reflect functional nucleosomal associations of DNA in vivo and are not predictable from the primary DNA sequence alone. Furthermore, TSS regions were distinguished from exonic and intronic by having the highest representation of subnucleosomal fragments (P<0.0001, FIG. 6c).

We next compared several other cfDNA fragmentation features for correlation with gene expression levels of peripheral blood leukocytes (FIG. 1d). While prior cfDNA profiling studies have reported lower depth of sequencing coverage at nucleosome depleted regions (NDR) within promoters of actively expressed genes, the correlation between PFE and expression was stronger than the correlation between normalized NDR depth and expression (FIG. 1b,d). Aside from the advantages of PFE for expression inferences made from cfDNA profiles using NDR depth at TSS regions, PFE also outperformed other previously defined fragmentomic metrics including windowed protection score (WPS), motif diversity score (MDS), and orientation-aware cfDNA fragmentation (OCF).

We next examined whether the distance from the TSS impacts correlations between cfDNA fragmentomic features and gene expression. When considering the 20 kb region flanking each promoter, we observed the peak correlation between cfDNA PFE and gene expression to be centered at the TSS. However, in comparison to NDR, correlation of PFE with gene expression had broader dispersion and extended into regions flanking the TSS (FIG. 1e). We also investigated the impact of sequencing depth on correlations between cfDNA fragmentomic signals and transcriptome-wide RNA expression. Interestingly, correlations plateaued around ˜500× sequencing depth (FIG. 1f). Overall, these results indicated that cfDNA fragmentation features are strongly correlated with RNA expression, and that PFE best captures this correlation compared to the other metrics studied.

We further confirmed our observations from WGS profiling of cfDNA by considering fragmentomic profiles within exonic regions, including fist exons adject to the TSS. Specifically, we profiled 5 cfDNA specimens—2 from a patient with small cell lung cancer (SCLC), 2 with castration-resistant prostate cancer (CRPC), and 1 from a healthy adult—by whole exome sequencing (WES) to target substantially higher depth (median unique coverage depth ˜2000×). Remarkably, individual genes known to be differentially expressed in these tumor types demonstrated the expected patterns of tumor-specific variation in their TSS regions (Methods). Indeed, SCLC- and CPRC-specific patterns were evident in the corresponding plasma cfDNA fragmentation profiles, including in AR and ASCL1, well-known genes for CRPC and SCLC, respectively (FIG. 1g). Nevertheless, these gene-level fragmentomic signals were discernable in the context of high tumor burdens (ctDNA >10%) of these patients, perhaps due to the partial representation of TSS regions that is inherent in the capture of first exons within WES.

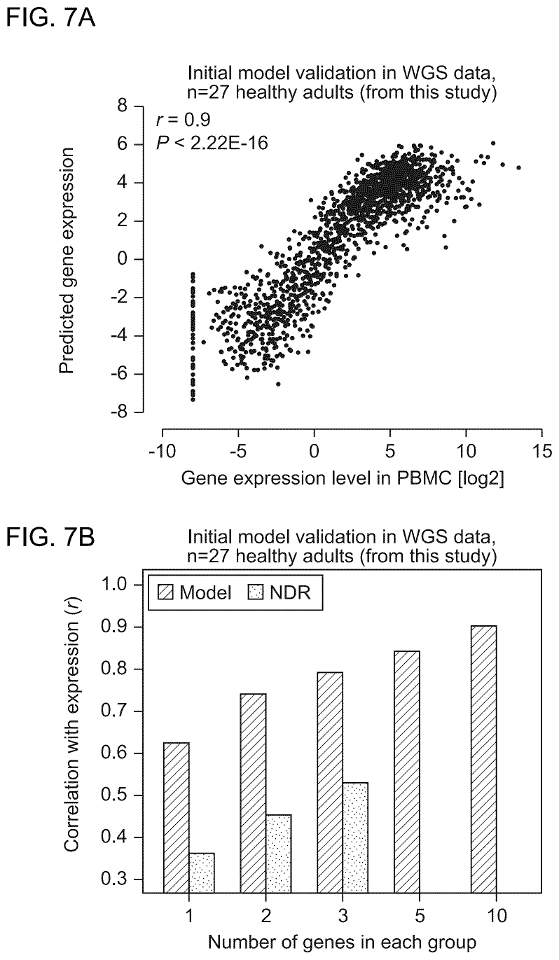

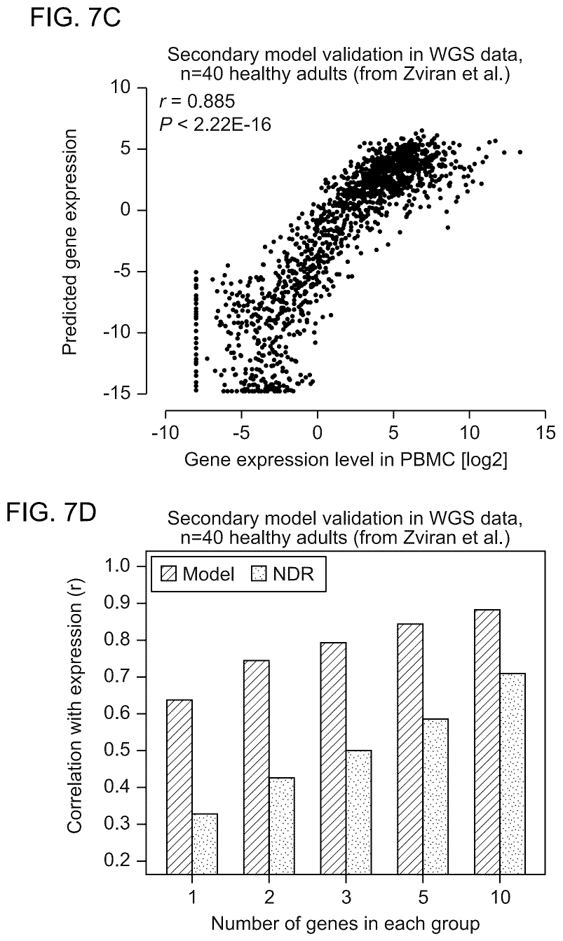

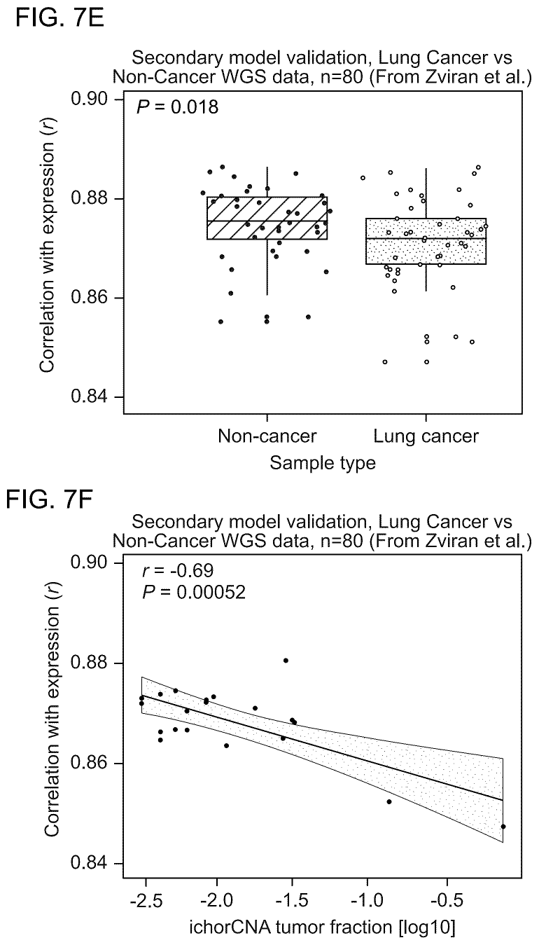

Inferring gene expression from cfDNA fragmentation profiles. We next attempted to predict gene expression from cfDNA fragmentomic features derived by WGS. When considering diverse fragmentomic metrics, we identified PFE and normalized NDR depth as complementary features predicting RNA expression in an ensemble generalized linear model (Methods). Specifically, while cfDNA fragmentomic features were loosely correlated to each other, PFE demonstrated better dynamic range for lowly expressed genes, while highly expressed genes appeared better captured by normalized NDR depth (FIG. 6d). We then validated this ensemble model by applying it to a fragmentomic ‘meta-profile’ assembled by WGS profiling of plasma cfDNA from 27 healthy adults (Methods). Here again we observed high correlation between model-predicted expression levels and observed measurements by RNA-Seq of PBMCs when considering groups of 10 genes (r=0.9, FIG. 7a). Consistent with our prior observations (FIG. 1f), these correlations deteriorated at lower sequencing depth in a manner that hampered resolution at the level of single genes (r=0.9 for 10-gene bins versus 0.79 for 3-gene bins versus 0.64 for individual TSSs; FIGS. 7a-b).

To validate the performance of our model in healthy versus cancer patients, we next re-analyzed genome-wide cfDNA profiling data from 40 healthy adults and 46 patients with early-stage lung cancers that were previously profiled by WGS at ˜20-40× coverage. We observed similar performance for predicting leukocyte gene expression levels when considering the average cfDNA meta-profile across the genome in the 40 healthy subjects (FIGS. 7c-d). When considering groups of 10 genes across the transcriptome, Pearson correlations between model predicted expression and expected RNA expression levels from PBMCs remained ˜0.85.

However, gene expression levels inferred from plasma cfDNA fragmentomic profiles of lung cancer patients were lower compared to PBMC transcriptomes (P=0.018; FIG. 7e). Hypothesizing that the lower correlation in lung cancer may be driven by an increased contribution of lung cancer-derived fragments, we used tumor fraction estimates by ichorCNA and observed a significant negative correlation with inferred leukocyte expression levels (r=−0.69, P=0.0005, FIG. 7f). This experiment demonstrates that tumor-derived cfDNA can substantially reduce the contribution of the leukocyte compartment to the cell-free nucleic acid pool, and this contribution can be measured by inferring tissue-specific gene expression from cfDNA when tumor burden is high.

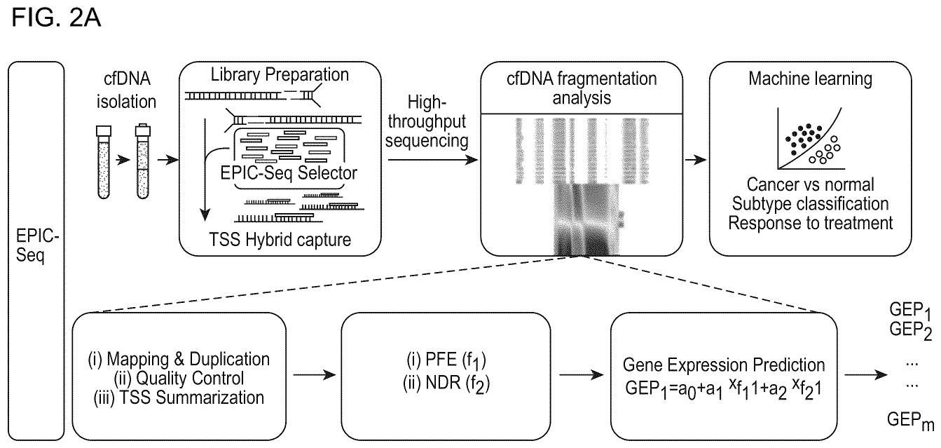

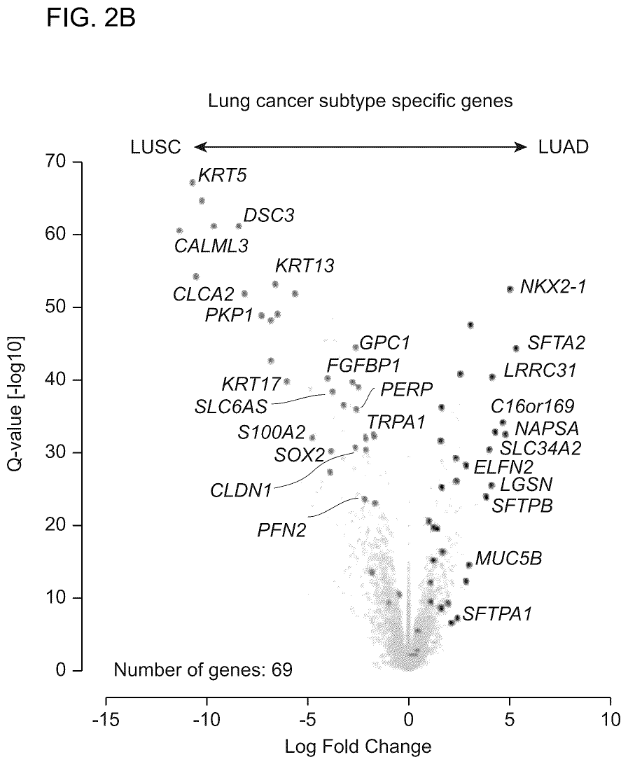

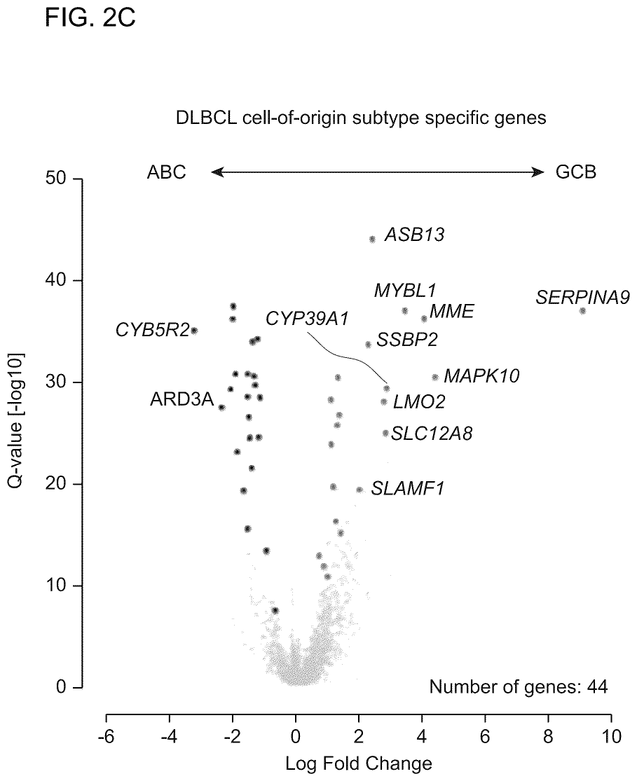

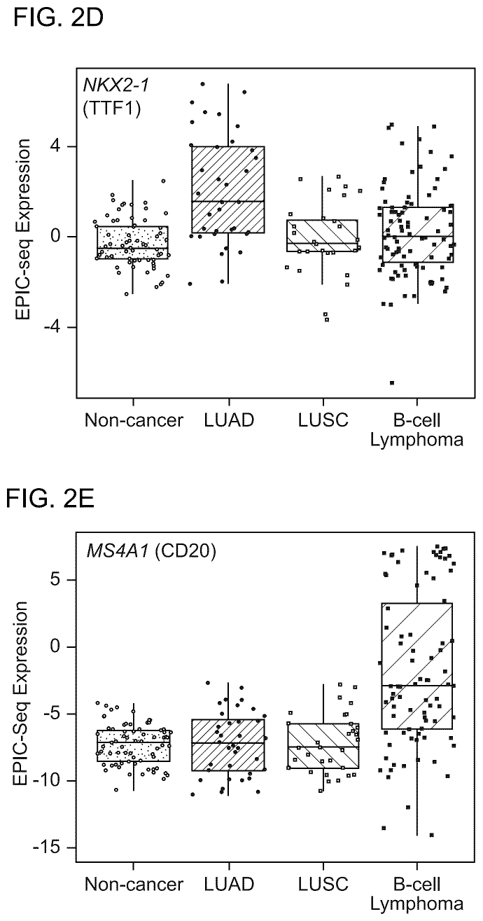

Epigenetic inference of expression by targeted deep cfDNA sequencing (EPIC-Seq). Based on our observation that PFE and NDR correlated better with gene expression at higher WGS sequencing depths (FIG. 1f), we next set out to develop a method allowing prediction of expression at the level of individual genes by deeper profiling of TSS regions. To do so, we devised a new approach—EPigenetic expression Inference from Cell-free DNA Sequencing (EPIC-Seq)—that combines hybrid capture-based targeted deep sequencing of TSS regions in cfDNA with machine learning for predicting RNA expression (FIG. 2a). The TSS regions targeted in an EPIC-Seq experiment are tailored to include genes expected to be differentially expressed in the conditions of interest (e.g., cancer versus normal, histologic subtype A vs subtype B, etc.)

We tested this framework by applying EPIC-Seq to two cancer classification problems using cfDNA: 1) noninvasively distinguishing histological subtypes of the most common solid tumor (Non-Small Cell Lung Cancer [NSCLC]), and 2) resolving molecular subtypes of the most common hematological malignancy (Diffuse Large B-Cell Lymphoma [DLBCL]). For each of these malignancies, we first identified genes highly expressed in tumor tissues, but with relatively low expression in whole blood (Methods). We then identified subtype-specific genes by evaluating those differentially expressed in NSCLC adenocarcinoma (LUAD) versus squamous cell carcinoma (LUSC) and DLBCL germinal center B-(GCB) versus activated B-cell (ABC) like subtypes. Specifically, we identified 69 differentially expressed genes (DEGs) when stratifying 1,156 NSCLC tumors by histological subtype from The Cancer Genome Atlas (TCGA; n=601 LUAD vs n=555 LUSC, FIG. 2b, Table 2). We separately identified 44 DEGs when stratifying 381 DLBCL tumors by molecular cell-of-origin (COO) subtype from prior publications (n=138 GCB vs n=243 ABC, FIG. 2c, Table 2). In addition to these 113 genes for classification of lung cancers and lymphoma subtypes, we also included 50 genes that are differentially expressed in leukocyte subsets as well as 16 genes as additional controls (Methods).

For each gene of interest, we designed probes to capture the ˜2 kb region flanking the TSS, then profiled plasma cfDNA from by deep sequencing of the targeted regions to a median ˜2,000× unique depth of coverage as previously described. In cfDNA fragmentomic profiles captured by WGS, we observed marginal gains in transcriptome wide correlations beyond ˜500× nominal coverage depth (FIG. 1f). Nevertheless, for our EPIC-Seq experiments and our modestly sized panel, we targeted ˜2000× unique depth (˜4-fold excess) for three reasons: (1) to guarantee saturation of the correlation plateau, (2) to avoid any gene-to-gene variability in accuracy of EPIC-Seq predictions of expression levels that might otherwise be attributable to spurious differences in depth variability due to non-uniform hybrid capture of the TSS regions of genes of interest, and (3) to address the lower partial concentration of cfDNA from non-hematopoietic tissues in circulation.

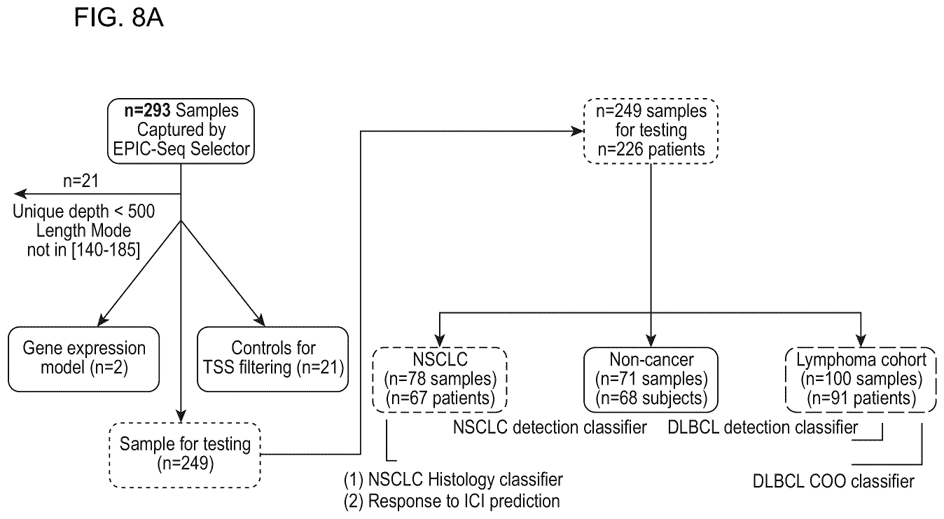

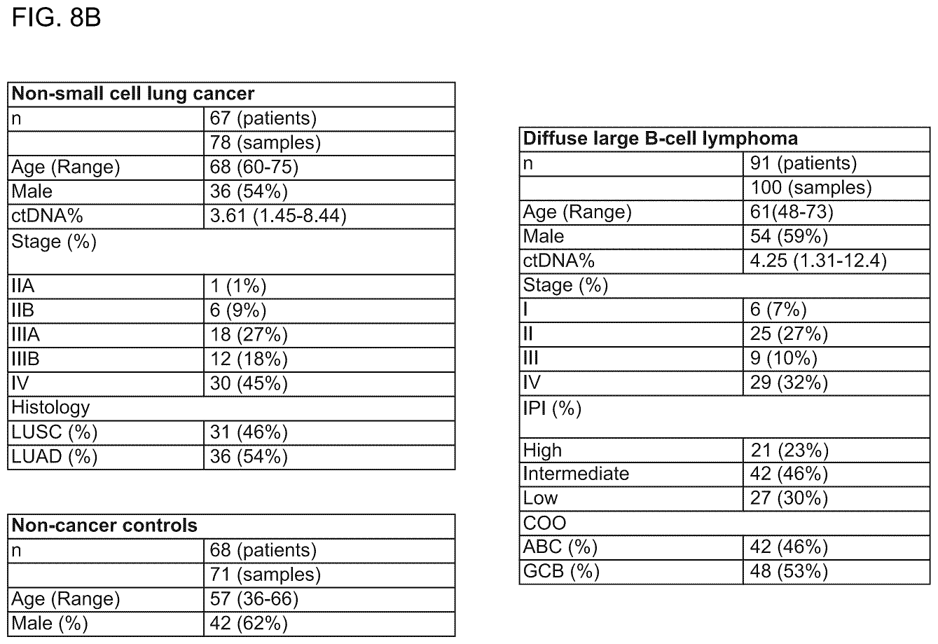

Using this workflow, we then profiled 307 plasma cfDNA samples, of which 263 were used for testing EPIC-Seq in different applications (FIG. 8a). This final set comprises 233 adults (FIG. 8a-b), including 67 patients with NSCLC (n=78 samples), 91 patients with DLBCL (n=100 samples), and 68 otherwise healthy subjects (n=71 samples). Using a custom EPIC-Seq analytical pipeline (Methods), we computed cfDNA fragmentomic features for each gene of interest, and then estimated its predicted RNA expression level (FIG. 2a). To explore the ability of EPIC-Seq to infer the expression of individual genes, we next evaluated expression of NKX2-1 (TTF1), a gene highly expressed in LUAD and useful in histopathological diagnosis, and MS4A1 (CD20), a gene highly expressed in DLBCL and useful for immunophenotyping and classification of lymphomas. Remarkably, the predicted expression level for NKX2-1 was significantly higher in plasma from patients with NSCLC-LUAD (Wilcoxon test P=4.2E−6; FIG. 2d). Conversely, the predicted expression level for MS4A1 was significantly higher in plasma from patients with DLBCL (Wilcoxon test P=4.2E−14; FIG. 2e). Collectively, these results demonstrate that inference of expression is accomplished by targeted deep cfDNA sequencing using EPIC-Seq, and that this framework can recover expected differences in tissue-derived expression at single-gene resolution.

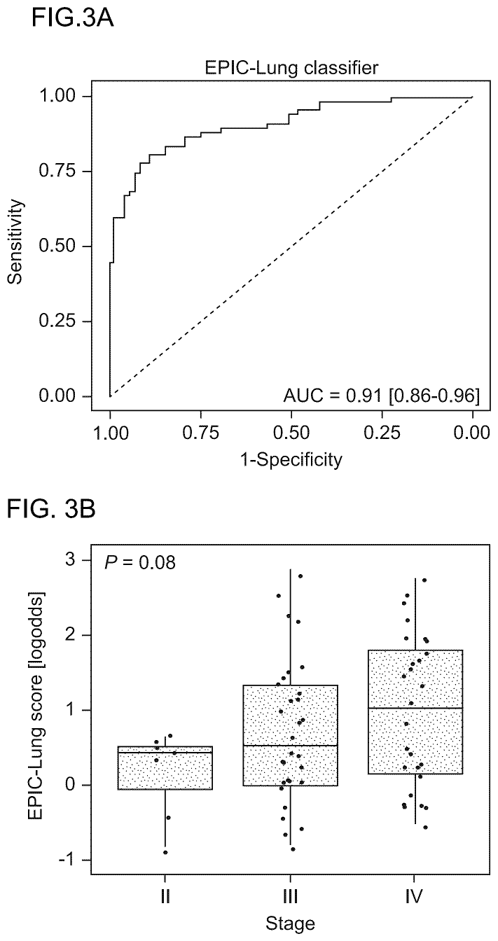

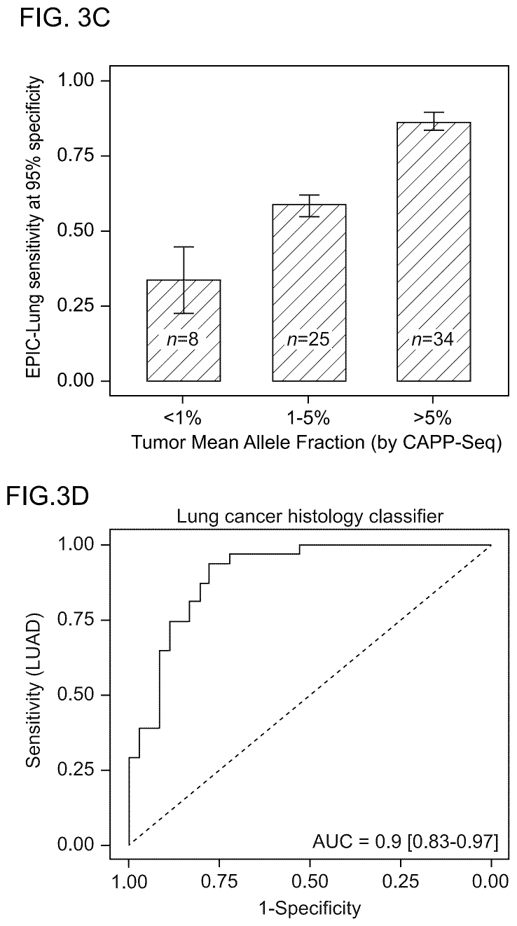

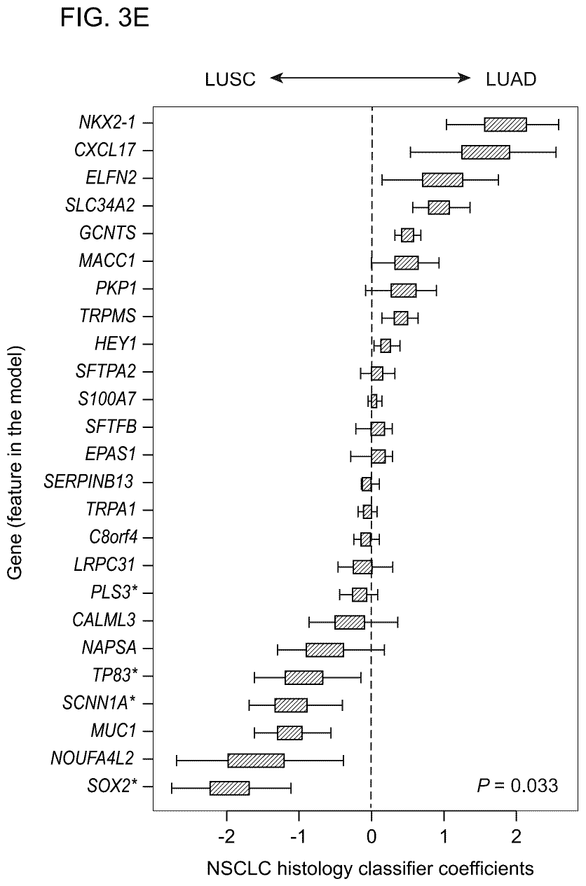

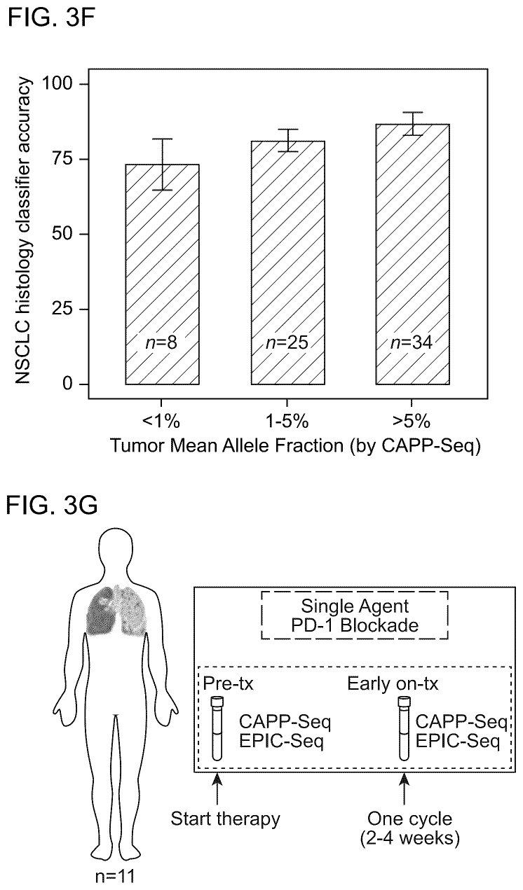

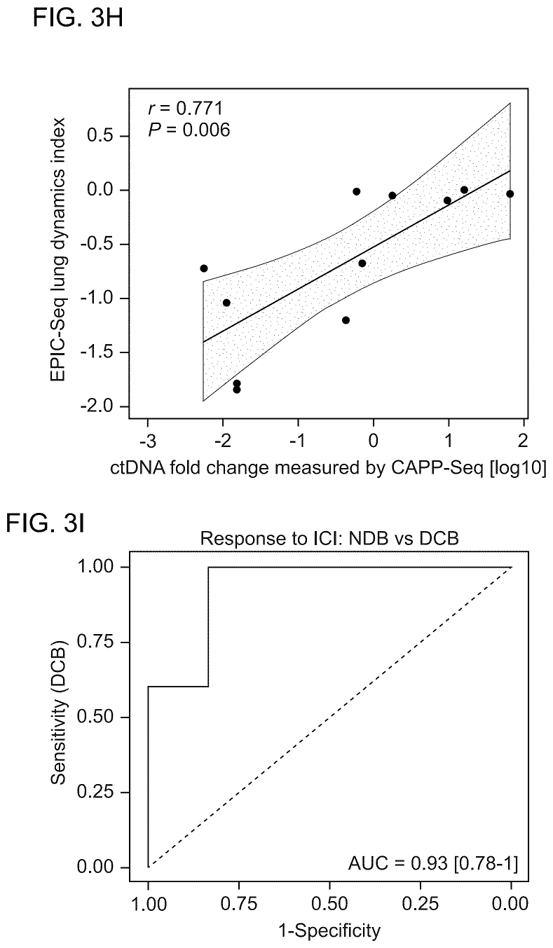

EPIC-Seq for lung cancer detection. We next evaluated whether EPIC-Seq might have utility for cancer classification problems, starting with lung cancer, the leading cause of cancer-related death in both men and women. We asked whether noninvasive classification of NSCLC cases versus healthy controls was feasible from cfDNA using EPIC-Seq. A classifier trained on EPIC-Seq data to distinguish NSCLC patients (n=67, stage II (n=7), stage III (n=30) and stage IV (n=30)) from non-cancer controls (n=71) revealed robust performance (EPIC-Lung AUC=0.91, 95% CI: 0.86-0.96 based on leave-one-out cross validation) when considering 141 TSS sites from 117 genes (FIG. 3a; Methods).

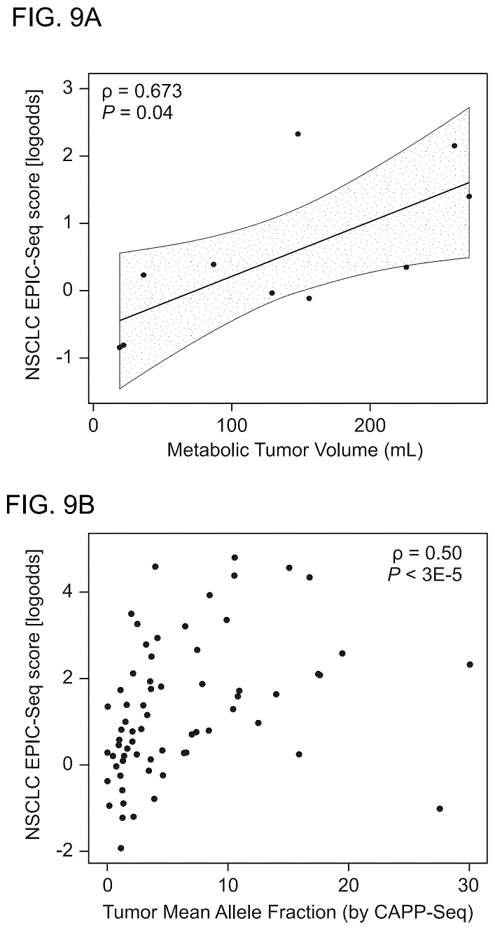

Epigenetic signals in cfDNA captured by our EPIC-Seq lung cancer classifier were significantly correlated with total metabolic tumor volumes (MTV), as measured by 18 Fluorodeoxyglucose (FDG) uptake in combined positron emission tomography and computed tomography studies (PET/CT; p=0.67; P=0.04; FIG. 9a), consistent with higher ctDNA concentrations in patients with larger tumor burdens. We also compared lung cancer epigenetic signals from EPIC-Seq in cfDNA with corresponding lung tumor-derived mutation signals from ctDNA separately measured by CAPP-Seq. Here again, EPIC-Seq lung signals in cfDNA seemed to capture tumor burden, as we observed significant correlation with the mean allelic fractions (AF) of tumor-derived somatic mutations measured by CAPP-Seq on the same specimens (ρ=0.5, P=3E−5; FIG. 9b). While most of the patients we profiled had advanced NSCLC, our classifier showed a statistical trend for stage III-IV cases having higher scores compared to stage II cases (P=0.08; FIG. 3b). We also assessed the importance of ctDNA concentration for the classifier's performance. When binning cases by ctDNA concentrations determined using mutations (CAPP-Seq), the EPIC-Seq lung classifier achieved ˜34% sensitivity at 95% specificity when allelic levels were below 1% and ˜86% sensitivity when ctDNA concentration exceeded 5% mean AF (FIG. 3c). These results collectively demonstrate that RNA expression from lung tumors inferred by EPIC-seq can distinguish lung cancer cases from non-cancer individuals and correlate with tumor burden.

Noninvasive classification of NSCLC subtypes. Adenocarcinomas (LUAD) and squamous cell carcinomas (LUSC) represent the two most common histological subtypes of NSCLC and differentiating between them is an important step in determining the optimal treatment for patients. Currently the morphologic and immunophenotypic criteria used for this classification are determined using tissue specimens, but invasive evaluation can be fraught by diagnostic challenges and by procedural risks. Importantly, to the best of our knowledge, currently available mutation-based liquid biopsy methods are unable to reliably distinguish between LUAD and LUSC.

We therefore asked whether such classification could be performed non-invasively using EPIC-Seq. In a cohort of 67 NSCLC patients, a regression classifier for distinguishing histological subtypes (LUAD n=36; LUSC n=31) was trained on EPIC-Seq data and demonstrated robust performance in cross-validation studies (AUC=0.90, 95% CI: 0.83-0.97; FIG. 3d; Methods). The genes with largest coefficients and therefore strongest impact on the classification included canonical markers for LUAD (SLC34A2, NKX2-1 [TTF1]) and LUSC (SOX2), thus confirming biological use of the classifier (Methods, FIG. 3e).

We evaluated the histology classifier's accuracy as a function of ctDNA levels as determined by CAPP-Seq (Methods) and as expected observed performance to be correlated with ctDNA concentration (FIG. 3f). Specifically, accuracy was highest at mean AFs above 5% (87%), with slight deterioration at levels between 1-5% (81%), and below 1% (73%) (FIG. 3f). These results demonstrate that inference of lung cancer expression differences by EPIC-seq allows for the noninvasive histological classification of NSCLC and that this framework appears robust across a range of ctDNA concentrations.

Predicting response to PD-(L)1 immune-checkpoint inhibition. For patients with advanced NSCLC, therapeutic blockade of programmed death 1 and programmed death-ligand 1 (PD-[L]1) signaling using monoclonal antibodies has shown remarkable promise. Trials combining PD-(L)1 blockade with cytotoxic therapy or with other immune checkpoint inhibition (ICI) strategies have demonstrated improved response rates at the risk of higher toxicity. Since only a minority of NSCLC patients achieve durable benefit from ICI, there is a critical unmet need for reliable biomarkers that can accurately identify these patients before or early during ICI therapy.

We therefore performed an exploratory analysis to test the biological plausibility of tracking fragmentomic features as informative for therapeutic response monitoring. Specifically, we tested whether early, non-invasive assessment of response to PD-(L)1 immune-checkpoint inhibitors might be feasible using EPIC-Seq. To do so, we analyzed 22 longitudinal blood specimens from 11 NSCLC patients treated with PD-(L)1 blockade using EPIC-Seq. Samples were collected immediately before PD-(L)1 therapy and within the first four weeks of therapy initiation (FIG. 3g). We developed a ‘lung dynamics index’ from EPIC-Seq predicted gene expression as a function of therapeutic benefit from ICI (Methods). This index demonstrated strong correlation to mutation-based response assessment using CAPP-Seq on the same specimens (r=0.77, P=0.006, FIG. 3h). The EPIC-seq lung dynamics index was also able to distinguish patients achieving durable clinical benefit (DCB; defined as no progression for at least 6 months after start of therapy) from those with no durable clinical benefit (NDB) achieving an AUC of 0.93, 95% CI: 0.78-1 (FIG. 3i). Of note, within the limitations of this small cohort, we also observed a significant and continuous association of EPIC-Seq classifier scores with progression-free survival (Wald P=0.046).

Noninvasive DLBCL quantitation using EPIC-Seq. Diffuse large B cell lymphoma (DLBCL) is the most common Non-Hodgkin's lymphoma (NHL) and displays remarkable clinical and biological heterogeneity. While aspects of this heterogeneity can be captured by clinical risk indices such as the International Prognostic Index, gene expression profiling, or genotyping of primary tumor biopsies, it remains unclear whether such stratification is feasible using less invasive approaches.

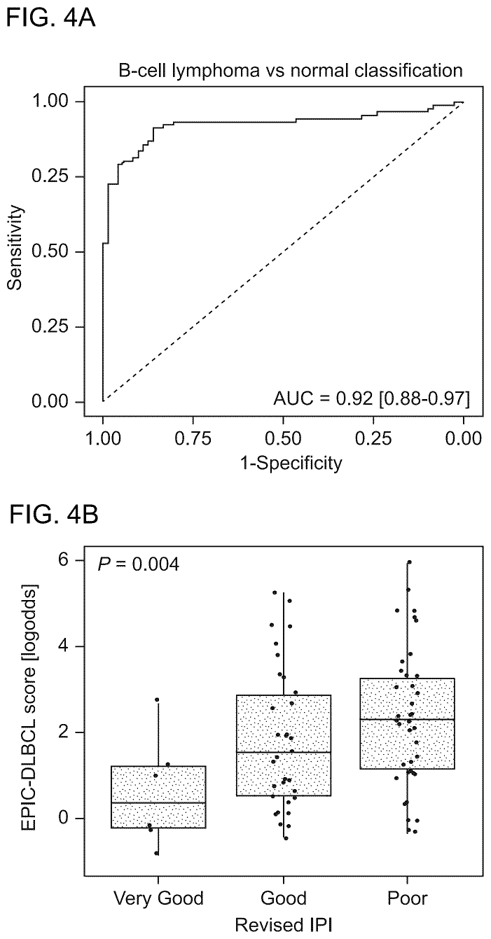

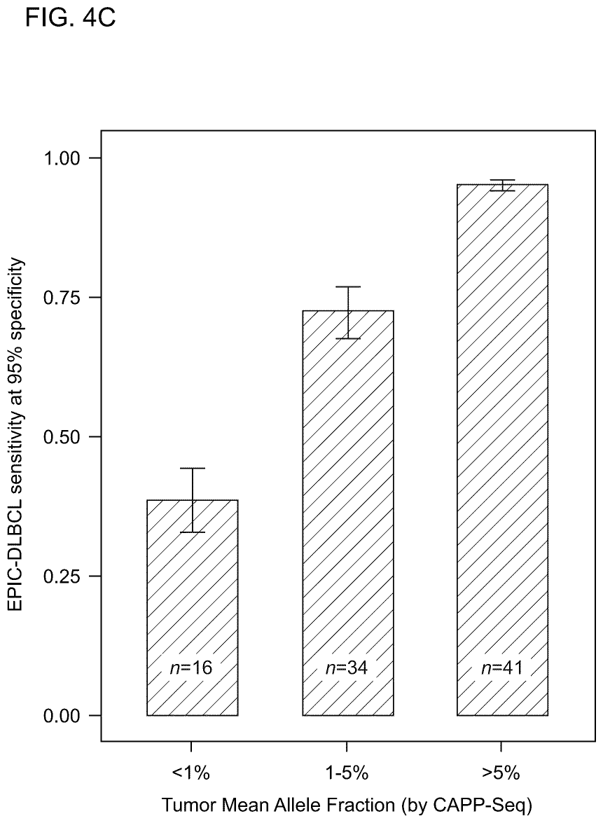

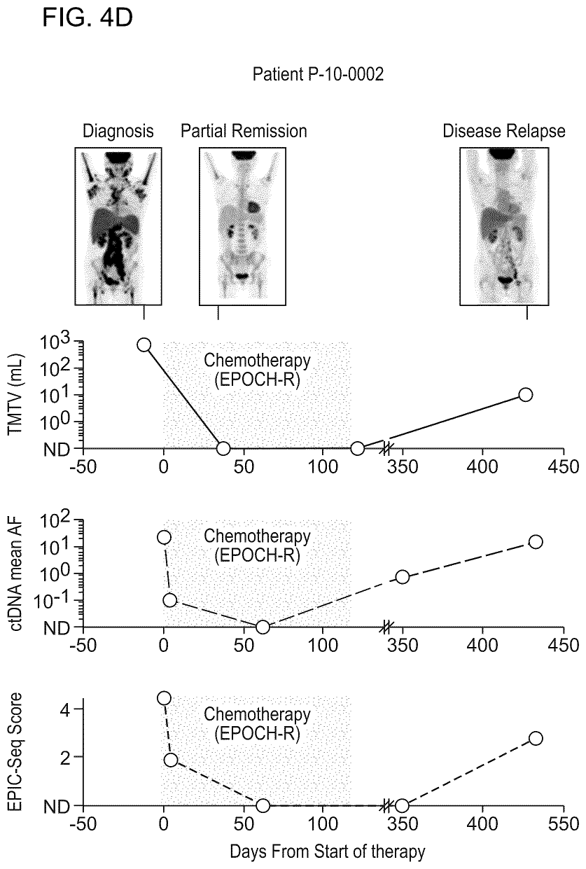

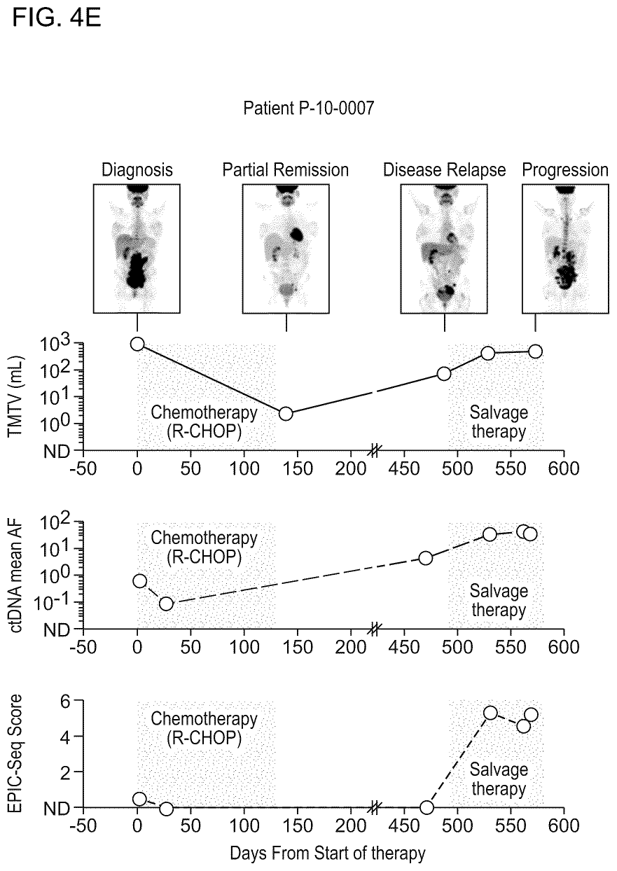

We therefore analyzed pre-treatment blood samples from DLBCL patients using EPIC-Seq and tested whether epigenetic signals in cfDNA allow noninvasive detection of DLBCL cases, distinguishing cancer patients from healthy controls. Here again, a regression classifier trained on EPIC-Seq data to distinguish DLBCL patients (n=91) from non-cancer controls (n=71) revealed robust performance (EPIC-DLBCL AUC=0.92, 95% CI 0.88-0.97 from leave-one-out cross validation; FIG. 4a; Methods). We observed a significant graded relationship between scores from this epigenetic classifier and the Revised International Prognostic Index (R-IPI; Jonckheere's trend test P=0.004; FIG. 4b). Separately, for patients with available PET/CT scans, we also observed a significant trend for scores from the epigenetic classifier in distinguishing patients with high versus low tumor burden as measured by total MTV (Wilcoxon P=0.015; FIG. 10a).

To further evaluate how EPIC-Seq scores reflect tumor burden in cfDNA, we compared them with the mean allele fractions (AFs) of mutations previously measured by CAPP-Seq on the same blood specimens. Notably, DLBCL epigenetic scores determined by EPIC-Seq were strongly correlated with the mean mutant AFs determined by CAPP-Seq (p=0.67, P<2E−16; FIG. 10b). We also evaluated the performance of our classifier at various ctDNA levels. Specifically, when trying to distinguish lymphoma cases from non-lymphoma subjects as controls and considering various mean AF thresholds determined by CAPP-Seq, we calculated the sensitivity for DLBCL detection at 95% specificity. While EPIC-Seq's sensitivity was strongly related to mean AF and showed most robust performance at ctDNA levels above 1%, we observed ˜40% detection of DLBCL cases where mean AF was below 1% before therapy (FIG. 4c).

To assess the relationship between epigenetic signals and somatic mutations during DLBCL therapy and their stability over time, we next profiled serial blood samples from 2 patients shortly after induction therapy with curative intent using both EPIC-Seq and CAPP-Seq (n=12; FIG. 4d-e). Again, we observed strong and significant correlations between DLBCL EPIC-Seq scores and ctDNA concentrations over time in both patients (ρ=0.79, P=0.004, FIG. 10c), despite the administration of combined chemoimmunotherapy and the substantial attendant changes in leukocyte blood counts. Collectively, these results illustrate that expression inferences by EPIC-seq can noninvasively detect tissue-derived DLBCL signals and faithfully reflect disease burden before and after DLBCL therapy.

DLBCL cell-of-origin classification. Most DLBCL tumors can be classified into two transcriptionally distinct molecular subtypes, each derived from a specific B cell differentiation state (cell of origin [COO]): germinal center B cell—like (GCB) and activated B cell—like (ABC). These subtypes are prognostic with significantly better outcomes observed in patients with GCB tumors, and may also predict sensitivity to emerging targeted therapies. While this classification of DLBCL is among the strongest prognostic factors and a potential biomarker for future personalized therapies, accurate subtyping remains challenging in clinical settings.

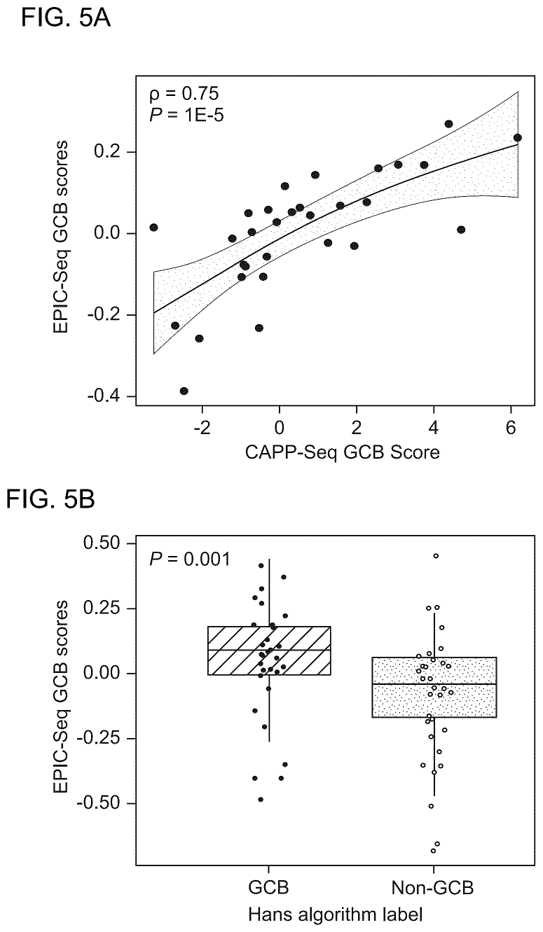

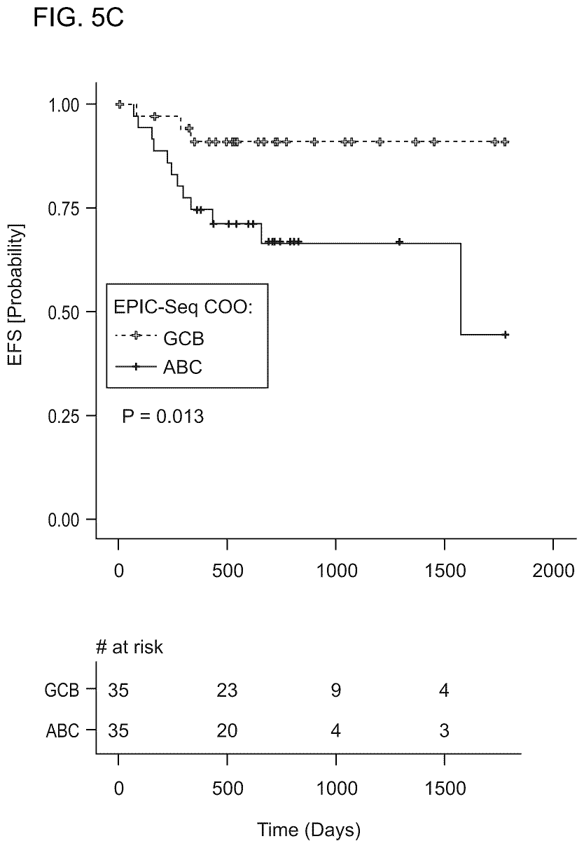

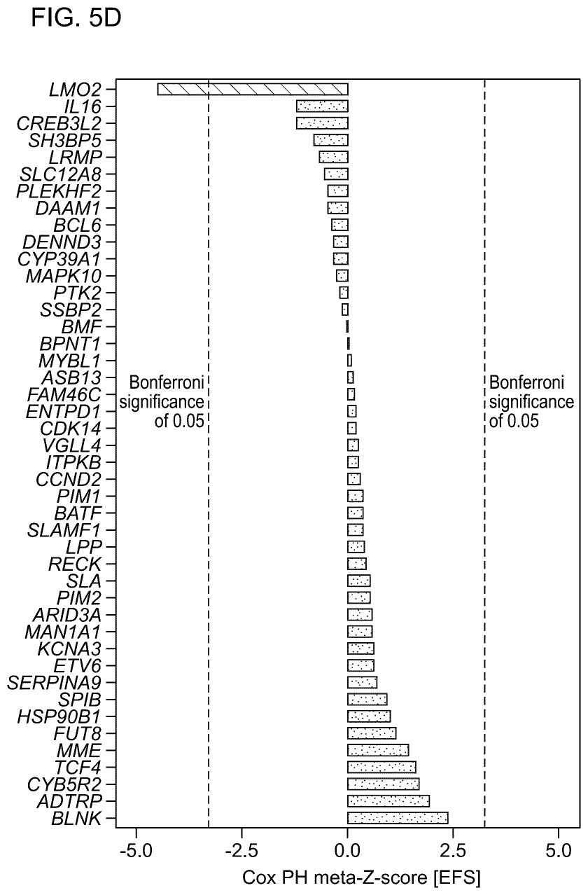

We therefore used EPIC-Seq profiling to develop a noninvasive COO classifier from pretreatment plasma. By considering differentially expressed genes in GCB or non-GCB (ABC) DLBCL and targeted by our panel, we built a probabilistic COO classifier similar to the ones described above (Methods). When we benchmarked this classifier's performance in our cohort of 90 DLBCL patients, we observed epigenetic scores to be significantly correlated with previously described mutation-based GCB scores (p=0.75, P=1E−5, FIG. 5a). When comparing patients classified by the more commonly clinically used immunohistochemical Hans classification algorithm, we observed a significantly higher COO score for GCB cases compared with Non-GCB (n=66, Wilcox P=0.001, FIG. 5b). Comparing the expected prognostic power of epigenetic and mutation-based COO scores using univariate Cox regressions, we observed a stronger association between EPIC-Seq GCB scores and favorable outcomes in the frontline therapy cases (n=70, EPIC-Seq: HR=0.13, P=0.033 vs CAPP-Seq: HR=0.95, P=0.62). Indeed, when stratified by the median GCB score in a Kaplan-Meier analysis, patients with higher GCB scores had significantly better outcomes (log-rank P=0.013, FIG. 5c). Among patients analyzed by both immunohistochemistry and DNA genotyping, the Hans algorithm failed to stratify patient clinical outcomes, demonstrating more accurate classification by our approach (FIG. 10d). Overall, these results show that EPIC-Seq has utility for noninvasive classification of DLBCL cell-of-origin and can stratify patients better than both the genetic COO classifier and the Hans algorithm.

Determining prognostic power of individual genes with EPIC-Seq. Expression profiling studies for a variety of tumor types have identified the prognostic power of individual genes for both risk stratification and therapeutic management. In DLBCL, prior studies have validated the prognostic utility of several key genes in relatively large patient populations that were homogenously treated with modern combination immune-chemotherapy using R-CHOP. These studies have relied on expression profiling from tumor biopsy specimens, which can be hampered by limitations of RNA sample quality and quantity.

Therefore, we wished to evaluate the utility of EPIC-Seq for noninvasively measuring expression of genes with prognostic associations in DLBCL. Using univariate Cox proportional hazard regression models, we tested the prognostic value of individual genes using pre-treatment blood plasma from 69 patients and used Z-scores to measure the relative strength of these associations. We first assessed the prognostic concordance of our results in blood plasma against primary tumor specimens by examining the correlation between our EPIC-Seq results with those described in 3 recent tumor expression profiling studies that relied on surgical DLBCL tissue specimens. When comparing the prognostic value of genes profiled in this manner, we observed a significant correlation of Z-scores from our study using plasma cfDNA with prior studies using tumor RNA (P=0.026; FIG. 10e).

Within our cohort, only LMO2 emerged as significantly associated with progression-free survival after correction for multiple hypothesis testing (nominal P=7.5E−6, corrected P=0.0055; FIG. 5d). This is consistent with prior data on its robust prognostic effect in DLBCL. LMO2 is an oncogene consisting of six exons, of which three nearest the 3′ end are protein coding. Inclusion of the three noncoding 5′ LMO2 exons is governed by alternative proximal, intermediate, and distal promoters. When comparing predicted expression from each of these alternative promoters for prognostic strength in DLBCL using EPIC-Seq, only the distal TSS (GRCh37/hg19-chr11:33,913,836) showed a significant association with outcome (FIG. 5e). Higher predicted expression from the distal TSS of LMO2 remained prognostic of more favorable outcomes in multivariable Cox regression after adjusting for IPI and ctDNA level (FIG. 5e). This result is consistent with the known importance of the distal LMO2 promoter in driving expression of LMO2 in human tumors, as evidenced by retroviral insertional mutagenic events observed in human gene therapy trials and chromosomal rearrangements mediating lymphomagenesis. Collectively, these observations indicate that EPIC-Seq has utility for noninvasively measuring the expression and prognostic value of individual genes and for resolving their individual TSS regions.

### Materials and Methods

Human subjects & Cohorts. Study overview. All samples analyzed in this study were collected with informed consent from subjects enrolled on Institutional Review Board-approved protocols complying with ethical regulations at their respective centers, as detailed below. Fragmentomic features used for EPIC-Seq were established and initially tested by profiling cfDNA through whole genome sequencing (WGS) and whole exome sequencing (WES), as tabulated in Table 1. These WGS and WES cfDNA profiling data derived from 125 subjects that were either generated for this study (n=30), or from publicly available datasets (n=95). For initial model development and cfDNA fragmentomic feature selection, we profiled cfDNA from a patient with carcinoma of unknown primary (CUP) by deep WGS at 2 time points (pre-treatment and relapse), from one patient with advanced SCLC (deep WES), and analyzed 9 cases with CRPC (WES). For initial validation analyses using WGS cfDNA fragmentomics, we reanalyzed samples from 67 healthy controls and 47 cancer patients previously described 15. After identification and initial validation of the key cfDNA fragmentomic signals informative for predicting gene expression in the 125 subjects described above by WGS/WES, EPIC-seq was then applied to 249 blood samples from 158 cancer patients and 68 healthy adults, as detailed below. To select genes for the EPIC-Seq capture panel, we analyzed publicly available gene expression datasets for 1156 lung cancers from The Cancer Genome Atlas and for 381 lymphomas from Schmitz et al., as described below.

Healthy subjects & Non-Cancer controls: To identify and validate cfDNA fragmentomic features informing gene expression prediction, WGS was performed in 27 healthy subjects. These subjects were profiled at varying pre-specified coverage depths (˜1-5×, n=24; ˜18-25×, n=3), thereby allowing construction of meta-profiles for expression inferences, as described below (see ‘Gene expression inference model’). We separately profiled 71 peripheral blood samples from 68 subjects without cancer using EPIC-Seq. Among these subjects, 20 (29%) qualified for lung cancer screening using low-dose CT (LDCT) due to a history of heavy smoking (≥30 pack years) and age (55-80 years).

### EPIC-Seq Cancer Cohorts

Lung Cancer Cohort: EPIC-Seq was applied to 78 blood samples from 67 patients diagnosed with NSCLC. Among these patients, 31 (46%) had a histological diagnosis of LUSC, while 36 (54%) patients had LUAD histology. Samples were collected at Stanford University, The University of Texas MD Anderson Cancer Center, or Memorial Sloan Kettering Cancer Centers, with patient characteristics outlined in FIG. 8b. A subset of patients with advanced NSCLC (n=11) was treated with PD-(L)1 blockade-based immune checkpoint inhibition and had serial pre- and on-treatment samples available. These patients had stage IV disease and were treated with PD-(L)1 blockade-based ICI.

DLBCL Cohort: EPIC-Seq was also applied to 100 samples from 91 patients diagnosed with large B-cell lymphoma. Samples were collected at Stanford Cancer Center, CA, USA; MD Anderson Cancer Center, TX, USA; Dijon, France; Novara, Italy; and within the Phase III multicenter PETAL trial, with baseline characteristics tabulated in FIG. 8b.

Patient with carcinoma of unknown primary (CUP): To assess with high resolution the relationship between fragmentomic features and gene expression we compared deep whole genome sequencing data and RNA-sequencing data of a patient with extremely low tumor burden. Tumor fraction was estimated using a tumor-informed plasma variant detection strategy. First, the patient's tumor germline DNA were prepared for exome capture using the Illumina Nextera Rapid Capture Exome Kit and sequenced on an Illumina Nextseq 500 machine using paired-end sequencing and 75-bp read lengths. Single nucleotide variant (SNV) calling was performed using Mutect and annotated by Annovar. A personalized targeted sequencing panel was generated using 120-bp IDT oligos overlapping SNVs detected in the tumor and applied to the tumor and germline sample. The variant set selected for monitoring consisted of 36 SNVs that both passed tumor/germline quality control filters and were present in at least 10% allele frequency in the tumor. The patient's plasma sample was sequenced on an Illumina NovaSeq machine, achieving a de-duplicated depth of 4000×. The time point used in this study had a monitoring mean allele frequency of 0.056% which is significantly lower than the lower limit of detection of disease at 250× coverage.

Clinical variables. Histopathology. Histological subtypes of each tumor type (NSCLC, DLBCL) profiled in this study were established according to clinical guidelines using microscopy and immunohistochemistry and served as ground truths for assessing classification performance by trained pathologists. COO subtypes of DLBCL were assessed based on the Hans classifier per WHO guidelines. For NSCLC and DLBCL subtypes profiled in prior studies by RNA-Seq, we relied on subtype labels from the TCGA (for LUAD vs LUSC subtypes of NSCLC) or from Schmitz el al. (for GCB vs ABC subtypes of DLBCL).

Metabolic tumor volume (MTV) measurement. Pre-treatment tumor MTV was measured from FDG PET/CT scans, using semiautomated software tools as previously described for NSCLC via MIM by using PETedge and DLBCL, respectively. Regional volumes were automatically identified by the software and confirmed by visual assessment of the expert to confirm inclusion of only pathological lesions.

Clinical Outcomes. Event-free survival (EFS) and overall survival (OS) were calculated from time of treatment initiation. OS events were death from any cause; EFS events were progression or relapse, unplanned retreatment of lymphoma and death resulting from any cause. Patients with NSCLC receiving PD(L)1 directed therapy were labeled as NDB or DCB for ‘experiencing progression or death’ and ‘durable clinical benefit’ within six months, respectively.

Specimen collection & Molecular profiling. Plasma collection & processing. Peripheral blood samples were collected in K₂EDTA or Streck Cell-Free DNA BCT tubes and processed according to local standards to isolate plasma before freezing. Following centrifugation, plasma was stored at −80° C. until cfDNA isolation. Cell-free DNA was extracted from 2 to 16 mL of plasma using the QlAamp Circulating Nucleic Acid Kit (Qiagen) according to the manufacturer's instructions. After isolation, cfDNA was quantified using the Qubit dsDNA High Sensitivity Kit (Thermo Fisher Scientific) and High Sensitivity NGS Fragment Analyzer (Agilent).

cfDNA sequencing library preparation. A median of 32 ng was input into library preparation. DNA input was scaled to control for high molecular weight DNA contamination. End repair, A-tailing, and custom adapter ligation containing molecular barcodes were performed following the KAPA Hyper Prep Kit manufacturer's instructions with ligation performed overnight at 4° C. as previously described. Shotgun cfDNA libraries were either subjected to whole genome sequencing (WGS) and/or subjected to hybrid capture of regions of interest as described below.

Hybrid capture & Sequencing. Exome capture: For Whole Exome Sequencing (WES), shotgun genomic DNA libraries were captured with the xGen Exome Research Panel v2 (IDT) per manufacturer's instructions with minor modifications. Hybridization was performed with 500 ng of each library in a single-plex capture for 16 hours at 65° C. After streptavidin bead washes and PCR amplification, post-capture PCR fragments were purified using the QlAquick PCR Purification Kit per manufacturer's instructions. Eluates were then further purified using a 1.5×AMPure XP bead cleanup.

Custom capture panels: We used CAPP-Seq to establish ctDNA levels, by genotyping of somatic variants including single nucleotide mutations. We used entity-specific CAPP-Seq capture panels for DLBCL or NSCLC (SeqCap EZ Choice, Roche NimbleGen), or personalized CAPP-Seq selectors for CUP (IDT), as previously described. Similarly, for EPIC-Seq, we used the SeqCap EZ Choice platform (Roche NimbleGen) to target TSS regions of genes of interest, as described below. Enrichment for WES, CAPP-Seq, and EPIC-Seq was done according to the manufacturers' protocols. Hybridization captures were then pooled, and multiplexed samples were sequenced on Illumina HiSeq4000 instruments as 2×150 bp reads.

RNA-Seq. The Illumina TruSeq RNA Exome kit was used for RNA-seq library preparation starting from 20 ng of input RNA, per manufacturer's instructions. When using peripheral blood as a source of leukocyte RNA, we used either plasma-depleted whole blood (PDWB) with globin depletion, or enriched PBMCs without globin depletion. In brief, total RNA was fragmented, and stranded cDNA libraries were created per the manufacturer's protocol. The RNA libraries were then enriched for the coding transcriptome by exon capture using biotinylated oligonucleotide baits. Hybridization captures were then pooled, and samples were sequenced on an Illumina HiSeq4000 as 2×150 bp lanes of 16-20 multiplexed samples per lane, yielding ˜20 million paired end reads per case. After demultiplexing, the data were aligned and expression levels summarized using Salmon to GENCODE version 27 transcript models. We separately studied tumor RNA-Seq data to identify differentially expressed genes of interest for EPIC-Seq panel design, as described in detail below.

Data analysis methods. Mapping, deduplication and quality control of TSS sites and sample. FASTQ files were demultiplexed using a custom pipeline wherein read pairs were considered only if both 8-bp sample barcodes and 6-bp UI Ds matched expected sequences after error-correction. After demultiplexing, barcodes were removed, and adaptor read-through was trimmed from the 3′ end of the reads using fastp to preserve short fragments. Fragments were aligned to human genome (hg19) using BWA; importantly, we disabled the automated distribution inference in BWA ALN to allow inclusion of shorter and longer cfDNA fragments that would otherwise be anomalously flagged as improperly paired. We removed PCR duplicates using a customized barcoding approach, which combines endogenous and exogenous unique molecular identifiers (UMIDs), including cfDNA fragment start and end positions, as well as pre-specified UMIDs within ligated adapters into account. To allow coverage uniformity for comparisons, we down-sampled data to 2000× depth using ‘samtools view-s’. Since in-silico simulations showed>500× sequencing depth to be required for achieving reasonable correlations between entropy and expression, we considered any samples not meeting this depth threshold (median depth) as failing quality control (QC). Any samples whose cfDNA fragment length density mode was below 140 or above 185 were also removed, since the expected fragment length density mode is 167 (corresponding to the chromatosomal DNA length). Together, these two criteria removed 21 samples as not meeting QC. To identify and censor noisy sites among the 236 TSS regions profiled by our EPIC-Seq panel, we profiled 23 controls (Table 2), allowing us to identify and remove stereotyped regions with reproducibly low TSS coverage (i.e., any site with CPM less than one third of uniformly distributed coverage across the TSSs in the selector, i.e.,

$$\frac{{10}^{6}}{236} \times \frac{1}{3} ,$$

in more than 75% of controls). This removed two TSS sites in FOXO1 and SFTA2 as not meeting QC.

To guarantee adequate quality of fragments entering analysis, we required mapping quality (MAPQ, k) of>30 or>10 in the WGS and EPIC-Seq data, respectively (using ‘samtools view-q k-F3084’). The more lenient EPIC-seq MAPQ threshold was qualified by more stringent mappability and uniqueness requirements already imposed on the TSS regions selected during EPIC-seq selector design. We also limited the analysis to reads with the following BAM FLAG set: 81, 93, 97, 99, 145, 147, 161, and 163. To ensure removal of non-unique fragments, reads with duplicate names were censored.

Fragmentomic feature extraction & summarization. We considered 5 cfDNA fragmentomic features at TSS regions and then compared each of these features to gene expression, including Window Protection Score (WPS), Orientation-aware CfDNA Fragmentation (OCF), Motif Diversity Score (MDS), Nucleosome depleted region score (NDR), and Promoter Fragmentation Entropy (PFE, introduced here). MDS, NDR, OCF, and WPS were each computed as per the conventions of the originally describing studies with minor modifications, as detailed below.

Motif diversity score (MDS). We performed end-motif sequence analysis of individual cfDNA fragments to assess the distribution of nucleotides among the first few positions for the reads of each read pair, as previously described. This was performed by computationally extracting the first four 5′ nucleotides of the genomic reference sequence for each sequence read, resulting in a 4-mer sequence motif. MDS was then computed as the Shannon index of the distribution across 256 motifs (4-mers) at each TSS site, when considering fragments overlapping the 2 kb window flanking each TSS. Of note, the first four 3′ nucleotides were not used as these may be altered by end-repair during library preparation and may not reflect the native genomic sequence.

Nucleosome depleted region score (NDR). To guard against variations in depth across the genome, including from GC-content variation or somatic copy number changes, depth was normalized within each 2-kilobase window flanking each TSS (−1000 to +1000 bp) in counts per million (CPM) space. We denote this normalized measure as nucleosome depleted region score, NDR, for each TSS.

### Promoter Fragmentation Entropy (PFE)

Shannon entropy was used to summarize the diversity in cfDNA fragment size values in the vicinity of each TSS site (−1 Kbps (upstream) to +1 Kbps (downstream)). We defined 201 size-bins [from b₁=100 bps to b₂₀₁=300 bps] and estimated the density by the maximum-likelihood, i.e., \hat{p}=[p₁, . . . , p₂₀₁] with

$$= \frac{{n}_{i}}{n}$$

where nᵢ and n denote the number of fragments with length bᵢ and total number of fragments at the TSS, respectively. Shannon's entropy was calculated as − [symbol] log₂ [symbol] and then normalized as follows. To account for variations in sequencing depth from sample to sample as well as other hidden factors impacting overall cfDNA fragment length distributions that might confound PFE, we defined a relative entropy using a Bayesian approach through a Dirichlet-multinomial model. In this model, fragment size profiles in a given cfDNA sample are assumed to follow a multinomial distribution (p) whose probability mass function is itself governed by a Dirichlet distribution, p˜Dirichlet(α), where vector α represents the parameter vector of the Dirichlet distribution. Here, we first used a set of genes to create a background fragment length density as α. For the background distribution, we focused on two flanking regions, (a) −1 Kbps (upstream) to −750 bps (upstream) and (b) from +750 bps (downstream) to +1 Kbps (downstream). The fragments that fell within those regions were used for the background fragment length distributions. We then randomly selected five background gene subsets and calculated their Shannon entropies, denoting these by e₁, e₂, e₃, e₄, and e₅. For a given TSS, we then calculated the posterior of the Dirichlet distribution, i.e., Dir(α*=α+[\hat{n}₁, . . . , \hat{n}₂₀₁]). The Shannon entropy of a given TSS was then compared with the five randomly generated entropies to measure the excess in diversity in the fragment length values at the TSS of interest. Formally, we define PFE as PFE(TSS):=Eₖ[Σ_{i:1-5}P*(e_{TSS}>(1+k)×eᵢ)] where Eₖ[.] denotes the expected value with respect to the excess parameter k, and P* is the probability with respect to the Dirichlet distribution Dir(α*). Here, we used a Gamma distribution for k˜Γ(s=0.5,r=1), where Γ is the Gamma distribution with shape s and rate r.

cfDNA fragmentomic analysis by WES profiling. Whole exome PFE analysis. For the whole exome analysis (in FIG. 1g), we used the raw Shannon entropy (as described in ‘Fragment length diversity calculation using Shannon entropy’) at any given gene, after transforming it into a z-score, using a cohort of 34 cfDNA WES profiles (each with 200-400× depth). To account for differences in depth in the cohort for normalization, we considered meta-profiles of 5 samples to achieve comparable depths as those initially used to relate PFE and gene expression levels when relying on WGS (2000×).

Small cell luno cancer Irene signature set. The SCLC gene signature was generated using an RNA-Seq data of 81 SCLC primary tumors. We performed differential gene expression analysis by comparing the RNA-seq data of these tumors with our reference PBMC RNA expression levels and identified genes in the top 1500 of SCLC expression overlapping genes in the bottom 5000 of the PBMC expression (‘high in SCLC’). Similarly, for ‘low in SCLC’ genes, we selected genes which are in top 1500 of PBMC expression and bottom 5,000 of SCLC expression. We further limited the gene set to those whose TSSs were covered in our whole exome panel to ensure sufficient sequencing coverage for analysis.

A gene expression model for predicting RNA output from TSS cfDNA fragmentomic features. To infer RNA expression levels from cfDNA fragmentation profiles at TSS regions of genes across the transcriptome, we built a prediction model using two features, PFE and NDR. Of note, among the 5 fragmentomic features considered, these indices demonstrate highest individual correlations as well as complementarity. For training, we employed one cfDNA sample sequenced to high coverage depth by WGS. We performed RNA-Seq on the PBMC of five healthy subjects and used the average across three of these individuals as the ‘reference expression vector’. Next, to achieve a higher resolution at the core promoters, we grouped every 10 genes, based on their expression in our reference RNA-seq vector. After removing genes used as background for calculating PFE, a total of 1,748 groups (of 10 genes each) remained. We then pooled all the fragments at the extended core promoters (−1 Kb/+1 Kb around the transcription start sites) of the genes within each group and extracted the two features: NDR and PFE. We then normalized the two features by 95% quantile over the background genes, where for PFE the normalization factor is

$$= min ( 1 , \frac{PFE}{Γ ( \frac{Q ( \{ PFE \} , 95 )}{{PFE}_{Bg}} ; 0.5 , 1 )} ) and = \frac{NDR}{{NDR}_{Bg}} ,$$

where Q(.,k) denotes the k^{th} quantile. By bootstrap resampling, we then built 600 ensemble models: 200 univariable PFE-alone-models m_{PFE,1}, m_{PFE,2}, . . . , m_{PFE,200}, 200 univariable NDR-alone-models m_{NDR,1}, m_{NDR,2}, . . . , m_{NDR,200} and 200 NDR-PFE integrated models m_{Int,1}, m_{Int,2}, . . . , m_{Int,200}.

To transfer this expression prediction model—which was originally derived from WGS—to the targeted TSS space (EPIC-seq), we evaluated each of the 600 models above, by measuring its root mean squared error (RMSE) on two held out healthy subjects. For each of these two healthy subjects, we compared the cfDNA profile by EPIC-seq to the corresponding PBMC transcriptome profile by RNA-Seq from the same blood specimen and computed the RMSE for each of the 600 ensemble models. The weight of each model was then proportionally scaled by the inverse RMSE of that model, with the final score then calculated as the linear sum of 600 models, weighted as described above.

EPIC-Seq panel design. Identification of cancer type-specific genes. We downloaded TCGA and DLBCL gene expression data in the form of RNA-Seq FPKM-UQ for all individuals using the GDC API. After removing samples from individuals with a history of more than one type of malignancy, we divided the remaining samples into two separate cohorts for training and validation (70% and 30% of each cancer type respectively). In the training set for each cancer type, median gene expression (FPKM-UQ) was calculated and protein coding genes in the upper 15th quantile were considered as highly expressed genes. To remove potentially confounding effects in cfDNA from variation in blood cells, we excluded genes within the upper 5^{th} quantile of expression in peripheral blood, when considering whole-blood transcriptome profiles from GTEx.

Gene selection for EPIC-Seq targeted sequencina panel design. We considered NSCLC and DLBCL, with known molecular subtypes exhibiting distinct gene expression profiles. Cancer-specific genes for LUAD, LUSC, and DLBCL were included. To find subtype-specific genes in NSCLC, we performed differential expression analysis using the DESeq2 package in R Bioconductor to distinguish LUAD and LUSC tumor transcriptomes from the TCGA. For the lymphoma analysis, a list of genes previously shown as differentially expressed between ABC and GCB subtypes according to RNA-Seq gene expression data was used. In addition to these DLBCL and NSCLC specific genes, we included 50 genes from the LM22 gene set capturing variation in peripheral blood leukocyte counts. Together these and other control genes contributed to a total of 179 unique genes, with each gene contributing one or more TSS regions to EPIC-Seq totaling 236 targeted TSS regions.

EPIC-Seq classification analyses and Machine Learning. Distinauishina lung cancer (EPIC-Lung classifier). The EPIC-Lung classifier was trained to distinguish lung cancer from non-cancer subjects. All the TSSs for immune cell type and NSCLC histology classification were used in this classifier. For genes with multiple TSS regions, in each iteration of cross-validation, we first combined TSS regions with intra-gene correlation exceeding 0.95 and capturing the mean. For those with correlation less than 0.95, we preserved individual TSS regions as independent reporters. This resulted in 139 features in the model and 143 samples (67 lung cancer cases and 71 controls). We then trained an [symbol] ₁- [symbol] ₂—regularized logistic regression model (‘elastic net’ with α=0.9) and an optimal λ obtained by cross-validation. The full model was evaluated through a leave-one-batch out (LOBO) model. Here, every batch contained at least one sample, and representing a set of samples that were either captured and/or sequenced together in one NGS sequencing lane.

Subclassification of NSCLC (EPIC-NSCLC-Subtype). A NSCLC histology subtype classifier was designed to distinguish the two major subtypes of non-small cell lung cancer, i.e., lung adenocarcinoma (LUAD) and lung squamous cell carcinoma (LUSC). Similar to the model in ‘EPIC-Lung classifier’, the classification model employs elastic net with α=0.9, with multiple TSS sites corresponding to one gene being merged. The performance of this classifier was evaluated via leave-one-out (LOO) analysis. The classifier was trained using 80 features with 67 samples (36 LUADs and 31 LUSCs). To evaluate performance, classification accuracy with equal weights was calculated.

Biological plausibility of classifier coefficients. We assessed the significance of the model coefficients in the NSCLC histology classifier from plasma cfDNA using EPIC-Seq and their concordance with prior design from tumor transcriptomes using RNA-Seq. Specifically, we compared nonzero coefficients from the elastic net model from cfDNA profiling, and then performed a t-test for the LUAD genes coefficients vs LUSC genes coefficients.

EPIC-seq lung dynamics score for the ICI treated patients. To predict benefit from immune checkpoint inhibitors, we first identified the differentially expressed TSSs in a discovery pre-treatment cohort (non-ICI; lung cancer vs normal). We then nominated the following TSS regions from genes with Bonferroni-corrected P<0.25 with a 1-sided t-test: (FOLR1 TSS#3, ITGA3 TSS#1, LRRC31 TSS#1, MACC1 TSS#1, NKX2-1 TSS#2, SCNN1A TSS#2, SFTPB TSS#1, WFDC2 TSS#1, CLDN1 TSS#1, FSCN1 TSS#1, GPC1 TSS#1, KRT17 TSS#1, PFN2 TSS#1 , PKP1 TSS#1, S100A2 TSS#1, SFN TSS#1 , SOX2TSS#2, TP63 TSS#2). Denoting the expression levels of these genes by ξₜ_{₀}=(x₁^{t}^{₀}, . . . , xₖ^{t}^{₀}) and ξₜ_{₁}=(x₁^{t}^{₁}, . . . , xₖ^{t}^{₁}) for time point t₀ and t₁, respectively, we defined (fold-change) statistics

$$s ( {ξ}_{{t}_{0}} , {ξ}_{{t}_{1}} ) = log \frac{\overline{{ξ}_{{t}_{1}}}}{\overline{{ξ}_{{t}_{0}}}}$$

where (̅.̅)̅ is used to denote averaging the vector elements. For each patient, we then empirically derived a null distribution for the s statistics by randomly selecting k sites from the EPIC-Seq selector. An empirical left-sided P-value was then calculated to measure response to therapy. The EPIC-seq dynamics score was then defined as the logarithm (base 10) of these empirical P-values.

Distinguishing lymphoma (EPIC-DLBCL classifier). This classifier was trained to distinguish DLBCL from non-cancer subjects using elastic-net, with regularization parameters being set as in ‘EPIC-Lung classifier’. The dataset used for LOBO cross-validation comprised 129 features and 167 samples (91 DLBCL cases and 71 controls).

Subclassification of DLBCL cell-of-origin (EPIC-DLBCL-COO). For the classification of DLBCL COO, we defined a GCB score as follows: (1) within a leave-one-out cross-validation framework, we first standardized each gene expression (i.e. the Z-score) and converted the Z-scores into probabilities, and then (2) defined a COO score as

$$- log 10 ( \frac{\sqrt[1 / \vert GCB \vert]{{\prod }_{i ∈ GCB} {p}_{i}}}{\sqrt[1 / \vert ABC \vert]{{\prod }_{i ∈ ABC} {p}_{i}}} ) .$$

Gene sets for each subtype were defined as originally selected in the EPIC-Seq selector design for DLBCL classification. To evaluate performance, we measured the concordance between EPIC-Seq scores and (1) genetic COO classification scores obtained from CAPP-Seq⁶², as well as (2) labels from Hans immunohistochemical algorithm.

Statistical and patient survival analysis. Associations between known and predicted variables were measured by Pearson correlation (r) or Spearman correlation (ρ) depending on data type. When data were normally distributed, group comparisons were determined using t-test with unequal variance or a paired t-test, as appropriate; otherwise, a two-sided Wilcoxon test was applied. To test for trend in continuous variables vs categorical groups, Jonckheere's trend test was used as implemented in the clinfun R package. Correction for multiple hypothesis testing was performed using the Bonferroni method. Results with two-sided P<0.05 were considered significant. Statistical analyses were performed with R 4.0.1. Confidence intervals (CI) are calculated by re-sampling with replacement (i.e., bootstrapping). Receiver operating characteristic (ROC) curve analyses were performed using the R package pROC. Survival analyses were performed using R package survival. When dichotomized, Kaplan-Meier estimates were used to plot the survival curves and statistical significance was evaluated by log-rank test. Otherwise, Cox proportional-hazards models were fitted to the data to determine the significance of each co-variate.

TABLE 1
Whole-genome (n = 114) and whole-exome (n = 11) sequencing of cell-free DNA samples were used
for the discovery of PFE, training the gene expression inference model and its validation. The WGS data
were either profiled in this study (n = 28) or downloaded from Zviran et al. (EGA accession number
EGAS00001004406). The WES data were either profiled in this study (n = 3) or downloaded from Adalsteinsson
et al. (dbGaP accession number phs001417.v1.p1). Cell-free DNA from 226 subjects were profiled using EPIC-seq.

| | | | | | | Non- | | | |
|---|---|---|---|---|---|---|---|---|---|
| | Sequencing | | | Subjects | Cancer | Cancer | | | |
| Cohort | platform | Subset | Purpose | (n) | Cases | Controls | FIGS. | Tables | Sources |
| Discovery | WGS | N/A | Feature | 114 | 47 | 67 | 1; | | This study |
| | | | discovery/ | | | | SF1/2 | | (n = 28); |
| | | | selection | | | | | | Zviran et al |
| | | | | | | | | | (n = 86) |
| Discovery/ | WES | N/A | Feature | 11 | 10 | 1 | 1 | | This study; |
| Validation | | | discovery/ | | | | | | Adalsteinsson |
| | | | validation | | | | | | et al (n = 9) |
| Validation | EPIC-Seq | EPIC | Disease | 67 | 67 | N/A | 2; 3 | | This study |
| | | Lung | detection, | | | | | | |
| | | | tumor | | | | | | |
| | | | classification, | | | | | | |
| | | | therapeutic | | | | | | |
| | | | response | | | | | | |
| Validation | EPIC-Seq | EPIC | Disease | 91 | 91 | N/A | 2; 4 | | This study |
| | | DLBCL | detection, | | | | | | |
| | | | tumor | | | | | | |
| | | | classification, | | | | | | |
| | | | therapeutic | | | | | | |
| | | | outcome | | | | | | |
| Validation | EPIC-Seq | EPIC | Non-Cancer | 68 | N/A | 68 | 2; 3; 4, 5 | | This study |
| | | Control | Controls | | | | | | |
| | | | (Specificity) | | | | | | |

TABLE 2
TSSs in the EPIC-seq selector. Each row corresponds to
one TSS in the EPIC-seq sequencing panel (‘selector’).

| | | | Gene | | |
|---|---|---|---|---|---|
| Hugo symbol | Chromosome | TSS [hg19] | strand | Category | TSS_ID |
| BPNT1 | chr1 | 220263191 | −1 | GCB | BPNT1_1 |
| FAM46C | chr1 | 118148603 | 1 | ABC | FAM46C_1 |
| ITPKB | chr1 | 226926876 | −1 | GCB | ITPKB_1 |
| KCNA3 | chr1 | 111217655 | −1 | ABC | KCNA3_1 |
| SLAMF1 | chr1 | 160617081 | −1 | GCB | SLAMF1_1 |
| CD1B | chr1 | 158301321 | −1 | Positive Control | CD1B_1 |
| CD1C | chr1 | 158259562 | 1 | Positive Control | CD1C_1 |
| CD1E | chr1 | 158323485 | 1 | Positive Control | CD1E_1 |
| CHI3L1 | chr1 | 203155922 | −1 | Positive Control | CHI3L1_1 |
| FCGR3B | chr1 | 161601252 | −1 | Positive Control | FCGR3B_1 |
| FCGR3B | chr1 | 161601753 | −1 | Positive Control | FCGR3B_2 |
| LCK | chr1 | 32716839 | 1 | Positive Control | LCK_1 |
| LCK | chr1 | 32739711 | 1 | Positive Control | LCK_2 |
| RGS13 | chr1 | 192605267 | 1 | Positive Control | RGS13_1 |
| BCL2L15 | chr1 | 114430169 | −1 | LUAD | BCL2L15_1 |
| MUC1 | chr1 | 155162706 | −1 | LUAD | MUC1_1 |
| CLCA2 | chr1 | 86889768 | 1 | LUSC | CLCA2_1 |
| IRF6 | chr1 | 209979520 | −1 | LUSC | IRF6_1 |
| PKP1 | chr1 | 201252579 | 1 | LUSC | PKP1_1 |
| S100A2 | chr1 | 153538306 | −1 | LUSC | S100A2_1 |
| S100A7 | chr1 | 153433137 | −1 | LUSC | S100A7_1 |
| SFN | chr1 | 27189632 | 1 | LUSC | SFN_1 |
| APOBEC4 | chr1 | 183622448 | −1 | negativeControl | APOBEC4_1 |
| TNNT2 | chr1 | 201346805 | −1 | negativeControl | TNNT2_1 |
| ASB13 | chr10 | 5708558 | −1 | GCB | ASB13_1 |
| BLNK | chr10 | 98031273 | −1 | ABC | BLNK_1 |
| BLNK | chr10 | 98031333 | −1 | ABC | BLNK_2 |
| ENTPD1 | chr10 | 97471535 | 1 | ABC | ENTPD1_1 |
| ENTPD1 | chr10 | 97515408 | 1 | ABC | ENTPD1_2 |
| ENTPD1 | chr10 | 97515672 | 1 | ABC | ENTPD1_3 |
| SFTPA1 | chr10 | 81370694 | 1 | LUAD | SFTPA1_1 |
| SFTPA2 | chr10 | 81320163 | −1 | LUAD | SFTPA2_{—} |
| SFTPD | chr10 | 81708861 | −1 | LUAD | SFTPD_1 |
| CALML3 | chr10 | 5566923 | 1 | LUSC | CALML3_1 |
| CYB5R2 | chr11 | 7694821 | −1 | ABC | CYB5R2_1 |
| LMO2 | chr11 | 33891371 | −1 | GCB | LMO2_1 |
| LMO2 | chr11 | 33891509 | −1 | GCB | LMO2_2 |
| LMO2 | chr11 | 33913836 | −1 | GCB | LMO2_3 |
| CXCR5 | chr11 | 118764100 | 1 | Positive Control | CXCR5_1 |
| MS4A1 | chr11 | 60223281 | 1 | Positive Control | MS4A1_1 |
| P2RY2 | chr11 | 72929343 | 1 | Positive Control | P2RY2_1 |
| P2RY2 | chr11 | 72929501 | 1 | Positive Control | P2RY2_2 |
| TYR | chr11 | 88911039 | 1 | Positive Control | TYR_1 |
| FOLR1 | chr11 | 71900601 | 1 | LUAD | FOLR1_1 |
| FOLR1 | chr11 | 71900958 | 1 | LUAD | FOLR1_2 |
| FOLR1 | chr11 | 71903172 | 1 | LUAD | FOLR1_3 |
| MUC5B | chr11 | 1244294 | 1 | LUAD | MUC5B_1 |
| MUC6 | chr11 | 1036706 | −1 | LUAD | MUC6_1 |
| TRIM29 | chr11 | 120008863 | −1 | LUSC | TRIM29_1 |
| CCND2 | chr12 | 4382901 | 1 | ABC | CCND2_1 |
| ETV6 | chr12 | 11802787 | 1 | ABC | ETV6_1 |
| HSP90B1 | chr12 | 104324188 | 1 | ABC | HSP90B1_1 |
| LRMP | chr12 | 25205180 | 1 | GCB | LRMP_1 |
| PMCH | chr12 | 102591614 | −1 | Positive Control | PMCH_1 |
| ST8SIA1 | chr12 | 22487648 | −1 | Positive Control | ST8SIA1_1 |
| SCNN1A | chr12 | 6484390 | −1 | LUAD | SCNN1A_1 |
| SCNN1A | chr12 | 6484905 | −1 | LUAD | SCNN1A_2 |
| SCNN1A | chr12 | 6486523 | −1 | LUAD | SCNN1A_3 |
| KRT5 | chr12 | 52914243 | −1 | LUSC | KRT5_1 |
| KRT6A | chr12 | 52887181 | −1 | LUSC | KRT6A_1 |
| NDUFA4L2 | chr12 | 57634475 | −1 | LUSC | NDUFA4L2_1 |
| FOXO1 | chr13 | 41240734 | −1 | DLBCLpath | FOXO1_1 |
| BATF | chr14 | 75988783 | 1 | ABC | BATF_1 |
| DAAM1 | chr14 | 59655380 | 1 | GCB | DAAM1_1 |
| DAAM1 | chr14 | 59730158 | 1 | GCB | DAAM1_2 |
| FUT8 | chr14 | 65877309 | 1 | ABC | FUT8_1 |
| FUT8 | chr14 | 65879447 | 1 | ABC | FUT8_2 |
| SERPINA9 | chr14 | 94942670 | −1 | GCB | SERPINA9_1 |
| GZMB | chr14 | 25103432 | −1 | Positive Control | GZMB_1 |
| GZMH | chr14 | 25078864 | −1 | Positive Control | GZMH_1 |
| TCL1A | chr14 | 96180533 | −1 | Positive Control | TCL1A_1 |
| NKX2-1 | chr14 | 36988903 | −1 | LUAD | NKX2-1_1 |
| NKX2-1 | chr14 | 36989430 | −1 | LUAD | NKX2-1_2 |
| RGS6 | chr14 | 72398816 | 1 | LUSC | RGS6_1 |
| RGS6 | chr14 | 72399155 | 1 | LUSC | RGS6_2 |
| RGS6 | chr14 | 72399785 | 1 | LUSC | RGS6_3 |
| BMF | chr15 | 40398287 | −1 | ABC | BMF_1 |
| BMF | chr15 | 40398639 | −1 | ABC | BMF_2 |
| BMF | chr15 | 40401075 | −1 | ABC | BMF_3 |
| L16 | chr15 | 81517639 | 1 | ABC | IL16_1 |
| AQP9 | chr15 | 58430407 | 1 | Positive Control | AQP9_1 |
| GCNT3 | chr15 | 59903981 | 1 | LUAD | GCNT3_1 |
| ITPKA | chr15 | 41786055 | 1 | LUAD | ITPKA_1 |
| IRF8 | chr16 | 85932773 | 1 | DLBCLpath | IRF8_1 |
| TPSAB1 | chr16 | 1290677 | 1 | Positive Control | TPSAB1_1 |
| C16orf89 | chr16 | 5116146 | −1 | LUAD | C16orf89_1 |
| MT1X | chr16 | 56716381 | 1 | LUSC | MT1X_1 |
| IKZF3 | chr17 | 38020441 | −1 | DLBCLpath | IKZF3_1 |
| ALOX15 | chr17 | 4544960 | −1 | Positive Control | ALOX15_1 |
| ITGA3 | chr17 | 48133339 | 1 | LUAD | ITGA3_1 |
| KRT13 | chr17 | 39661865 | −1 | LUSC | KRT13_1 |
| KRT15 | chr17 | 39675270 | −1 | LUSC | KRT15_1 |
| KRT16 | chr17 | 39769079 | −1 | LUSC | KRT16_1 |
| KRT17 | chr17 | 39780882 | −1 | LUSC | KRT17_1 |
| ANKFN1 | chr17 | 54230835 | 1 | negativeControl | ANKFN1_1 |
| MYL4 | chr17 | 45286713 | 1 | negativeControl | MYL4_1 |
| TCF4 | chr18 | 52969852 | −1 | ABC | TCF4_1 |
| TCF4 | chr18 | 52989090 | −1 | ABC | TCF4_2 |
| TCF4 | chr18 | 53071226 | −1 | ABC | TCF4_3 |
| TCF4 | chr18 | 53089723 | −1 | ABC | TCF4_4 |
| TCF4 | chr18 | 53178000 | −1 | ABC | TCF4_5 |
| TCF4 | chr18 | 53255860 | −1 | ABC | TCF4_6 |
| TCF4 | chr18 | 53257045 | −1 | ABC | TCF4_7 |
| DSC3 | chr18 | 28622781 | −1 | LUSC | DSC3_1 |
| DSG3 | chr18 | 29027731 | 1 | LUSC | DSG3_1 |
| SERPINB13 | chr18 | 61254533 | 1 | LUSC | SERPINB13_1 |
| ARID3A | chr19 | 926036 | 1 | ABC | ARID3A_1 |
| SPIB | chr19 | 50922194 | 1 | ABC | SPIB_1 |
| TCF3 | chr19 | 1650286 | −1 | DLBCLpath | TCF3_1 |
| CLC | chr19 | 40228669 | −1 | Positive Control | CLC_1 |
| FFAR2 | chr19 | 35940616 | 1 | Positive Control | FFAR2_1 |
| NKG7 | chr19 | 51875960 | −1 | Positive Control | NKG7_1 |
| CXCL17 | chr19 | 42947136 | −1 | LUAD | CXCL17_1 |
| ICAM1 | chr19 | 10381516 | 1 | LUAD | ICAM1_1 |
| NAPSA | chr19 | 50868931 | −1 | LUAD | NAPSA_1 |
| SLC1A6 | chr19 | 15083730 | −1 | LUSC | SLC1A6_1 |
| CCL20 | chr2 | 228678557 | 1 | Positive Control | CCL20_1 |
| CTLA4 | chr2 | 204732510 | 1 | Positive Control | CTLA4_1 |
| ICOS | chr2 | 204801470 | 1 | Positive Control | ICOS_1 |
| ZAP70 | chr2 | 98330030 | 1 | Positive Control | ZAP70_1 |
| ZAP70 | chr2 | 98350868 | 1 | Positive Control | ZAP70_2 |
| EPAS1 | chr2 | 46524540 | 1 | LUAD | EPAS1_1 |
| SFTPB | chr2 | 85895864 | −1 | LUAD | SFTPB_1 |
| TRPM8 | chr2 | 234826042 | 1 | LUAD | TRPM8_1 |
| GPC1 | chr2 | 241375114 | 1 | LUSC | GPC1_1 |
| ALPP | chr2 | 233243347 | 1 | negativeControl | ALPP_1 |
| WFDC2 | chr20 | 44098393 | 1 | LUAD | WFDC2_1 |
| IGLL3P | chr22 | 25714223 | 1 | Positive Control | IGLL3P_1 |
| ELFN2 | chr22 | 37823505 | −1 | LUAD | ELFN2_1 |
| BCL6 | chr3 | 187452695 | −1 | GCB | BCL6_1 |
| BCL6 | chr3 | 187454285 | −1 | GCB | BCL6_2 |
| BCL6 | chr3 | 187463513 | −1 | GCB | BCL6_3 |
| LPP | chr3 | 187871662 | 1 | GCB | LPP_1 |
| LPP | chr3 | 187930720 | 1 | GCB | LPP_2 |
| LPP | chr3 | 187943192 | 1 | GCB | LPP_3 |
| MME | chr3 | 154797435 | 1 | GCB | MME_1 |
| MME | chr3 | 154797704 | 1 | GCB | MME_2 |
| MME | chr3 | 154797952 | 1 | GCB | MME_3 |
| MME | chr3 | 154798078 | 1 | GCB | MME_4 |
| SH3BP5 | chr3 | 15374136 | −1 | ABC | SH3BP5_1 |
| SLC12A8 | chr3 | 124930243 | −1 | GCB | SLC12A8_1 |
| SLC12A8 | chr3 | 124931609 | −1 | GCB | SLC12A8_2 |
| VGLL4 | chr3 | 11610398 | −1 | GCB | VGLL4_1 |
| VGLL4 | chr3 | 11623836 | −1 | GCB | VGLL4_2 |
| VGLL4 | chr3 | 11762220 | −1 | GCB | VGLL4_3 |
| FOXP1 | chr3 | 71114074 | −1 | DLBCLpath | FOXP1_1 |
| FOXP1 | chr3 | 71180092 | −1 | DLBCLpath | FOXP1_2 |
| FOXP1 | chr3 | 71294316 | −1 | DLBCLpath | FOXP1_3 |
| FOXP1 | chr3 | 71353911 | −1 | DLBCLpath | FOXP1_4 |
| FOXP1 | chr3 | 71592708 | −1 | DLBCLpath | FOXP1_5 |
| FOXP1 | chr3 | 71632904 | −1 | DLBCLpath | FOXP1_6 |
| FOXP1 | chr3 | 71633140 | −1 | DLBCLpath | FOXP1_7 |
| CPA3 | chr3 | 148583042 | 1 | Positive Control | CPA3_1 |
| GPR171 | chr3 | 150920988 | −1 | Positive Control | GPR171_1 |
| HESX1 | chr3 | 57234280 | −1 | Positive Control | HESX1_1 |
| P2RY13 | chr3 | 151047337 | −1 | Positive Control | P2RY13_1 |
| P2RY14 | chr3 | 150966998 | −1 | Positive Control | P2RY14_1 |
| P2RY14 | chr3 | 150996230 | −1 | Positive Control | P2RY14_2 |
| LRRC31 | chr3 | 169587660 | −1 | LUAD | LRRC31_1 |
| CLDN1 | chr3 | 190040235 | −1 | LUSC | CLDN1_1 |
| PFN2 | chr3 | 149688741 | −1 | LUSC | PFN2_1 |
| SOX2 | chr3 | 181328150 | 1 | LUSC | SOX2_1 |
| SOX2 | chr3 | 181429711 | 1 | LUSC | SOX2_2 |
| TP63 | chr3 | 189349215 | 1 | LUSC | TP63_1 |
| TP63 | chr3 | 189507448 | 1 | LUSC | TP63_2 |
| MAPK10 | chr4 | 87028806 | −1 | GCB | MAPK10_1 |
| MAPK10 | chr4 | 87281375 | −1 | GCB | MAPK10_2 |
| MAPK10 | chr4 | 87374283 | −1 | GCB | MAPK10_3 |
| BANK1 | chr4 | 102711763 | 1 | Positive Control | BANK1_1 |
| BANK1 | chr4 | 102734982 | 1 | Positive Control | BANK1_2 |
| CXCL3 | chr4 | 74904490 | −1 | Positive Control | CXCL3_1 |
| CXCL5 | chr4 | 74864416 | −1 | Positive Control | CXCL5_1 |
| HPGDS | chr4 | 95264027 | −1 | Positive Control | HPGDS_1 |
| LEF1 | chr4 | 109087953 | −1 | Positive Control | LEF1_1 |
| LEF1 | chr4 | 109090112 | −1 | Positive Control | LEF1_2 |
| LEF1-AS1 | chr4 | 109088680 | 1 | Positive Control | LEF1-AS1_1 |
| LEF1-AS1 | chr4 | 109093275 | 1 | Positive Control | LEF1-AS1_2 |
| SLC34A2 | chr4 | 25657434 | 1 | LUAD | SLC34A2_1 |
| SLC34A2 | chr4 | 25658085 | 1 | LUAD | SLC34A2_2 |
| FGFBP1 | chr4 | 15940363 | −1 | LUSC | FGFBP1_1 |
| SSBP2 | chr5 | 81047072 | −1 | GCB | SSBP2_1 |
| GZMA | chr5 | 54398473 | 1 | Positive Control | GZMA_1 |
| GZMK | chr5 | 54320106 | 1 | Positive Control | GZMK_1 |
| IL3 | chr5 | 131396346 | 1 | Positive Control | IL3_1 |
| IL9 | chr5 | 135231516 | −1 | Positive Control | IL9_1 |
| TCF7 | chr5 | 133450401 | 1 | Positive Control | TCF7_1 |
| TCF7 | chr5 | 133451297 | 1 | Positive Control | TCF7_2 |
| TCF7 | chr5 | 133451349 | 1 | Positive Control | TCF7_{—} |
| SCGB3A2 | chr5 | 147258273 | 1 | LUAD | SCGB3A2_1 |
| ADTRP | chr6 | 11779280 | .1 | ABC | ADTRP_1 |
| CYP39A1 | chr6 | 46620523 | −1 | GCB | CYP39A1_1 |
| MAN1A1 | chr6 | 119670931 | −1 | ABC | MAN1A1_1 |
| PIM1 | chr6 | 37137921 | 1 | ABC | PIM1_1 |
| IRF4 | chr6 | 391738 | 1 | DLBCLpath | IRF4_1 |
| TREM2 | chr6 | 41130922 | −1 | Positive Control | TREM2_1 |
| VNN2 | chr6 | 133084598 | −1 | Positive Control | VNN2_1 |
| ENPP3 | chr6 | 131958441 | 1 | LUAD | ENPP3_1 |
| LGSN | chr6 | 64029882 | −1 | LUAD | LGSN_1 |
| SFTA2 | chr6 | 30899952 | −1 | LUAD | SFTA2_1 |
| FABP7 | chr6 | 123100645 | 1 | LUSC | FABP7_1 |
| PERP | chr6 | 138428660 | −1 | LUSC | PERP_1 |
| CDK14 | chr7 | 90338711 | 1 | GCB | CDK14_1 |
| CREB3L2 | chr7 | 137686847 | −1 | ABC | CREB3L2_1 |
| EGFR | chr7 | 55086724 | 1 | amplificationControl | EGFR_1 |
| MET | chr7 | 116312458 | 1 | amplificationControl | MET_1 |
| MACC1 | chr7 | 20181538 | 1 | LUAD | MACC1_1 |
| MACC1 | chr7 | 20257013 | −1 | LUAD | MACC1_2 |
| FSCN1 | chr7 | 5632435 | 1 | LUSC | FSCN1_1 |
| GPNMB | chr7 | 23286315 | 1 | LUSC | GPNMB_1 |
| HOXA1 | chr7 | 27135625 | −1 | LUSC | HOXA1_1 |
| AGMO | chr7 | 15601640 | −1 | negativeControl | AGMO_1 |
| MYL7 | chr7 | 44180916 | −1 | negativeControl | MYL7_1 |
| DENND3 | chr8 | 142138719 | 1 | GCB | DENND3_1 |
| MYBL1 | chr8 | 67525480 | −1 | GCB | MYBL1_1 |
| PLEKHF2 | chr8 | 96145948 | 1 | GCB | PLEKHF2_1 |
| PTK2 | chr8 | 142011412 | −1 | GCB | PTK2_1 |
| SLA | chr8 | 134072603 | −1 | ABC | SLA_1 |
| SLA | chr8 | 134115310 | −1 | ABC | SLA_2 |
| MYC | chr8 | 128748314 | 1 | amplificationControl | MYC_1 |
| BLK | chr8 | 11351520 | 1 | Positive Control | BLK_1 |
| C8orf4 | chr8 | 40010986 | 1 | LUAD | C8orf4_1 |
| HEY1 | chr8 | 80680098 | −1 | LUSC | HEY1_1 |
| TRPA1 | chr8 | 72987819 | −1 | LUSC | TRPA1_1 |
| RECK | chr9 | 36036909 | 1 | GCB | RECK |
| CCL19 | chr9 | 34691274 | −1 | Positive Control | CCL19_1 |
| CD72 | chr9 | 35618424 | −1 | Positive Control | CD72_1 |
| FCN1 | chr9 | 137809806 | −1 | Positive Control | FCN1_1 |
| AQP3 | chr9 | 33447631 | −1 | LUAD | AQP3_1 |
| GOLM1 | chr9 | 88714510 | −1 | LUAD | GOLM1_1 |
| GOLM1 | chr9 | 88715116 | −1 | LUAD | GOLM1_2 |
| PIM2 | chrX | 48776413 | −1 | ABC | PIM2_1 |
| CLIC2 | chrX | 154563986 | −1 | Positive Control | CLIC2_1 |
| HMGB3 | chrX | 150151762 | 1 | LUAD | HMGB3_1 |
| PLS3 | chrX | 114795176 | 1 | LUAD | PLS3_1 |
| PLS3 | chrX | 114827818 | 1 | LUAD | PLS3_2 |
| SLC6A8 | chrX | 152954965 | 1 | LUSC | SLC6A8_1 |

### References

1. Jahr, S. et al. DNA fragments in the blood plasma of cancer patients: quantitations and evidence for their origin from apoptotic and necrotic cells. Cancer Res 61, 1659-1665 (2001).
2. Lo, Y.M. et al. Maternal plasma DNA sequencing reveals the genome-wide genetic and mutational profile of the fetus. Sci Transl Med 2, 61ra91 (2010).
3. Heitzer, E., Auinger, L. & Speicher, M. R. Cell-Free DNA and Apoptosis: How Dead Cells Inform About the Living. Trends Mol Med 26, 519-528 (2020).
4. Newman, A. M. et al. An ultrasensitive method for quantitating circulating tumor DNA with broad patient coverage. Nat Med 20, 548-554 (2014).
5. Phallen, J. et al. Direct detection of early-stage cancers using circulating tumor DNA. Sci Transl Med 9 (2017).
6. Cohen, J. D. et al. Detection and localization of surgically resectable cancers with a multi-analyte blood test. Science 359, 926-930 (2018).
7. Cristiano, S. et al. Genome-wide cell-free DNA fragmentation in patients with cancer. Nature 570, 385-389 (2019).
8. Heitzer, E., Hague, I. S., Roberts, C. E. S. & Speicher, M. R. Current and future perspectives of liquid biopsies in genomics-driven oncology. Nat Rev Genet 20, 71-88 (2019).
9. Chabon, J. J. et al. Integrating genomic features for non-invasive early lung cancer detection. Nature 580, 245-251 (2020).
10. Van Opstal, D. et al. Origin and clinical relevance of chromosomal aberrations other than the common trisomies detected by genome-wide NIPS: results of the TRIDENT study. Genet Med 20, 480-485 (2018).
11. Fan, H. C. et al. Non-invasive prenatal measurement of the fetal genome. Nature 487, 320-324 (2012).
12. Knight, S. R., Thorne, A. & Lo Faro, M. L. Donor-specific Cell-free DNA as a Biomarker in Solid Organ Transplantation. A Systematic Review. Transplantation 103, 273-283 (2019).
13. Chaudhuri, A. A. et al. Early Detection of Molecular Residual Disease in Localized Lung Cancer by Circulating Tumor DNA Profiling. Cancer Discov 7, 1394-1403 (2017).
14. Lennon, A. M. et al. Feasibility of blood testing combined with PET-CT to screen for cancer and guide intervention. Science 369 (2020).
15. Zviran, A. et al. Genome-wide cell-free DNA mutational integration enables ultra-sensitive cancer monitoring. Nat Med 26, 1114-1124 (2020).
16. Lo, Y. M. et al. Presence of donor-specific DNA in plasma of kidney and liver-transplant recipients. Lancet 351, 1329-1330 (1998).
17. Snyder, T. M., Khush, K. K., Valantine, H. A. & Quake, S. R. Universal noninvasive detection of solid organ transplant rejection. Proc Natl Acad Sci U S A 108, 6229-6234 (2011).
18. Lehmann-Werman, R. et al. Identification of tissue-specific cell death using methylation patterns of circulating DNA. Proc Natl Acad Sci U S A 113, E1826-1834 (2016).
19. Jiang, P. et al. Preferred end coordinates and somatic variants as signatures of circulating tumor DNA associated with hepatocellular carcinoma. Proc Natl Acad Sci U S A 115, E10925-E10933 (2018).
20. Sun, K. et al. Orientation-aware plasma cell-free DNA fragmentation analysis in open chromatin regions informs tissue of origin. Genome Res 29, 418-427 (2019).
21. Sadeh, R. et al. ChIP-seq of plasma cell-free nucleosomes identifies gene expression programs of the cells of origin. Nat Biotechnol (2021).
22. Lui, Y. Y. et al. Predominant hematopoietic origin of cell-free DNA in plasma and serum after sex-mismatched bone marrow transplantation. Clin Chem 48, 421-427 (2002).
23. Fleischhacker, M. & Schmidt, B. Circulating nucleic acids (CNAs) and cancer--a survey. Biochim Biophys Acta 1775, 181-232 (2007).
24. Ramachandran, S., Ahmad, K. & Henikoff, S. Transcription and Remodeling Produce Asymmetrically Unwrapped Nucleosomal Intermediates. Mol Cell 68, 1038-1053 e1034 (2017).
25. Snyder, M. W., Kircher, M., Hill, A. J., Daza, R. M. & Shendure, J. Cell-free DNA Comprises an In Vivo Nucleosome Footprint that Informs Its Tissues-Of-Origin. Cell 164, 57-68 (2016).
26. Ivanov, M., Baranova, A., Butler, T., Spellman, P. & Mileyko, V. Non-random fragmentation patterns in circulating cell-free DNA reflect epigenetic regulation. BMC Genomics 16 Suppl 13, S1 (2015).
27. Ulz, P. et al. Inferring expressed genes by whole-genome sequencing of plasma DNA. Nat Genet 48, 1273-1278 (2016).
28. Wu, J. et al. Decoding genetic and epigenetic information embedded in cell free DNA with adapted SALP-seq. Int J Cancer 145, 2395-2406 (2019).
29. Jiang, P. et al. Lengthening and shortening of plasma DNA in hepatocellular carcinoma patients. Proc Natl Acad Sci U S A 112, E1317-1325 (2015).
30. Underhill, H. R. et al. Fragment Length of Circulating Tumor DNA. PLoS Genet 12, e1006162 (2016).
31. Mouliere, F. et al. Enhanced detection of circulating tumor DNA by fragment size analysis. Sci Transl Med 10 (2018).
32. Ulz, P. et al. Inference of transcription factor binding from cell-free DNA enables tumor subtype prediction and early detection. Nat Commun 10, 4666 (2019).
33. Moss, J. et al. Comprehensive human cell-type methylation atlas reveals origins of circulating cell-free DNA in health and disease. Nat Commun 9, 5068 (2018).
34. Weintraub, H. & Groudine, M. Chromosomal subunits in active genes have an altered conformation. Science 193, 848-856 (1976).
35. Jiang, P. et al. Plasma DNA End-Motif Profiling as a Fragmentomic Marker in Cancer, Pregnancy, and Transplantation. Cancer Discov 10, 664-673 (2020).
36. Cancer Genome Atlas Research, N. Comprehensive molecular profiling of lung adenocarcinoma. Nature 511, 543-550 (2014).
37. Cancer Genome Atlas Research, N. Comprehensive genomic characterization of squamous cell lung cancers. Nature 489, 519-525 (2012).
38. Schmitz, R. et al. Genetics and Pathogenesis of Diffuse Large B-Cell Lymphoma. N Engl J Med 378, 1396-1407 (2018).
39. Newman, A. M. et al. Robust enumeration of cell subsets from tissue expression profiles. Nat Methods 12, 453-457 (2015).
40. Newman, A. M. et al. Integrated digital error suppression for improved detection of circulating tumor DNA. Nat Biotechnol 34, 547-555 (2016).
41. Maloney, D. G. et al. Phase I clinical trial using escalating single-dose infusion of chimeric anti-CD20 monoclonal antibody (IDEC-C2B8) in patients with recurrent B-cell lymphoma. Blood 84, 2457-2466 (1994).
42. Puglisi, F. et al. Prognostic value of thyroid transcription factor-1 in primary, resected, non-small cell lung carcinoma. Mod Pathol 12, 318-324 (1999).
43. Ferlay, J. et al. Cancer incidence and mortality worldwide: sources, methods and major patterns in GLOBOCAN 2012. Int J Cancer 136, E359-386 (2015).
44. Torre, L. A., Siegel, R. L. & Jemal, A. Lung Cancer Statistics. Adv Exp Med Biol 893, 1-19 (2016).
45. Travis, W. D. et al. The 2015 World Health Organization Classification of Lung Tumors: Impact of Genetic, Clinical and Radiologic Advances Since the 2004 Classification. J Thorac Oncol 10, 1243-1260 (2015).
46. Reck, M. & Rabe, K. F. Precision Diagnosis and Treatment for Advanced Non-Small-Cell Lung Cancer. N Engl J Med 377, 849-861 (2017).
47. Ettinger, D. S. et al. NCCN Guidelines Insights: Non-Small Cell Lung Cancer, Version 1.2020. J Natl Compr Canc Netw 17, 1464-1472 (2019).
48. Wiener, R. S., Schwartz, L. M., Woloshin, S. & Welch, H. G. Population-based risk for complications after transthoracic needle lung biopsy of a pulmonary nodule: an analysis of discharge records. Ann Intern Med 155, 137-144 (2011).
49. Bubendorf, L., Lantuejoul, S., de Langen, A. J. & Thunnissen, E. Nonsmall cell lung carcinoma: diagnostic difficulties in small biopsies and cytological specimens: Number 2 in the Series “Pathology for the clinician” Edited by Peter Dorfmuller and Alberto Cavazza. Eur Respir Rev 26 (2017).
50. McLean, A. E. B., Barnes, D. J. & Troy, L. K. Diagnosing Lung Cancer: The Complexities of Obtaining a Tissue Diagnosis in the Era of Minimally Invasive and Personalised Medicine. J Clin Med 7 (2018).
51. Reck, M. et al. Pembrolizumab versus Chemotherapy for PD-L1-Positive Non-Small-Cell Lung Cancer. N Engl J Med 375, 1823-1833 (2016).
52. Socinski, M. A. et al. Atezolizumab for First-Line Treatment of Metastatic Nonsquamous NSCLC. N Engl J Med 378, 2288-2301 (2018).
53. Gandhi, L. et al. Pembrolizumab plus Chemotherapy in Metastatic Non-Small-Cell Lung Cancer. N Engl J Med 378, 2078-2092 (2018).
54. Hellmann, M. D. et al. Nivolumab plus Ipilimumab in Lung Cancer with a High Tumor Mutational Burden. N Engl J Med 378, 2093-2104 (2018).
55. Camidge, D. R., Doebele, R. C. & Kerr, K. M. Comparing and contrasting predictive biomarkers for immunotherapy and targeted therapy of NSCLC. Nat Rev Clin Oncol 16, 341-355 (2019).
56. Nabet, B. Y. et al. Noninvasive Early Identification of Therapeutic Benefit from Immune Checkpoint Inhibition. Cell 183, 363-376 e313 (2020).
57. Menon, M. P., Pittaluga, S. & Jaffe, E. S. The histological and biological spectrum of diffuse large B-cell lymphoma in the World Health Organization classification. Cancer J 18, 411-420 (2012).
58. Sehn, L. H. et al. The revised International Prognostic Index (R-IPI) is a better predictor of outcome than the standard IPI for patients with diffuse large B-cell lymphoma treated with R-CHOP. Blood 109, 1857-1861 (2007).
59. Alizadeh, A. A. et al. Distinct types of diffuse large B-cell lymphoma identified by gene expression profiling. Nature 403, 503-511 (2000).
60. Pasqualucci, L. et al. Analysis of the coding genome of diffuse large B-cell lymphoma. Nat Genet 43, 830-837 (2011).
61. Cottereau, A. S. et al. Molecular Profile and FDG-PET/CT Total Metabolic Tumor Volume Improve Risk Classification at Diagnosis for Patients with Diffuse Large B-Cell Lymphoma. Clin Cancer Res 22, 3801-3809 (2016).
62. Scherer, F. et al. Distinct biological subtypes and patterns of genome evolution in lymphoma revealed by circulating tumor DNA. Sci Transl Med 8, 364ra155 (2016).
63. Kurtz, D. M. et al. Circulating Tumor DNA Measurements As Early Outcome Predictors in Diffuse Large B-Cell Lymphoma. J Clin Oncol 36, 2845-2853 (2018).
64. Rosenwald, A. et al. The use of molecular profiling to predict survival after chemotherapy for diffuse large-B-cell lymphoma. N Engl J Med 346, 1937-1947 (2002).
65. Basso, K. & Dalla-Favera, R. Germinal centres and B cell lymphomagenesis. Nat Rev Immunol 15, 172-184 (2015).
66. Dunleavy, K. et al. Differential efficacy of bortezomib plus chemotherapy within molecular subtypes of diffuse large B-cell lymphoma. Blood 113, 6069-6076 (2009).
67. Thieblemont, C. et al. The germinal center/activated B-cell subclassification has a prognostic impact for response to salvage therapy in relapsed/refractory diffuse large B-cell lymphoma: a bio-CORAL study. J Clin Oncol 29, 4079-4087 (2011).
68. Scott, D. W. et al. Determining cell-of-origin subtypes of diffuse large B-cell lymphoma using gene expression in formalin-fixed paraffin-embedded tissue. Blood 123, 1214-1217 (2014).
69. Nowakowski, G. S. et al. Lenalidomide combined with R-CHOP overcomes negative prognostic impact of non-germinal center B-cell phenotype in newly diagnosed diffuse large B-Cell lymphoma: a phase II study. J Clin Oncol 33, 251-257 (2015).
70. Wilson, W. H. et al. Targeting B cell receptor signaling with ibrutinib in diffuse large B cell lymphoma. Nat Med 21, 922-926 (2015).
71. Young, R. M. & Staudt, L. M. Targeting pathological B cell receptor signalling in lymphoid malignancies. Nat Rev Drug Discov 12, 229-243 (2013).
72. Lenz, G. et al. Stromal gene signatures in large-B-cell lymphomas. N Engl J Med 359, 2313-2323 (2008).
73. Zelenetz, A. D. et al. NCCN Guidelines Insights: B-Cell Lymphomas, Version 3.2019. J Natl Compr Canc Netw 17, 650-661 (2019).
74. Hans, C. P. et al. Confirmation of the molecular classification of diffuse large B-cell lymphoma by immunohistochemistry using a tissue microarray. Blood 103, 275-282 (2004).
75. Lossos, I. S. et al. Prediction of survival in diffuse large-B-cell lymphoma based on the expression of six genes. N Engl J Med 350, 1828-1837 (2004).
76. Malumbres, R. et al. Paraffin-based 6-gene model predicts outcome in diffuse large B-cell lymphoma patients treated with R-CHOP. Blood 111, 5509-5514 (2008).
77. Alizadeh, A. A., Gentles, A. J., Lossos, I. S. & Levy, R. Molecular outcome prediction in diffuse large-B-cell lymphoma. N Engl J Med 360, 2794-2795 (2009).
78. Alizadeh, A. A. et al. Prediction of survival in diffuse large B-cell lymphoma based on the expression of 2 genes reflecting tumor and microenvironment. Blood 118, 1350-1358 (2011).
79. Chapuy, B. et al. Molecular subtypes of diffuse large B cell lymphoma are associated with distinct pathogenic mechanisms and outcomes. Nat Med 24, 679-690 (2018).
80. Ennishi, D. et al. Double-Hit Gene Expression Signature Defines a Distinct Subgroup of Germinal Center B-Cell-Like Diffuse Large B-Cell Lymphoma. J Clin Oncol 37, 190-201 (2019).
81. Gentles, A. J. & Alizadeh, A. A. A few good genes: simple, biologically motivated signatures for cancer prognosis. Cell Cycle 10, 3615-3616 (2011).
82. Chambers, J. & Rabbitts, T. H. LMO2 at 25 years: a paradigm of chromosomal translocation proteins. Open Biol 5, 150062 (2015).
83. Royer-Pokora, B. et al. The TTG-2/RBTN2 T cell oncogene encodes two alternative transcripts from two promoters: the distal promoter is removed by most 11p13 translocations in acute T cell leukaemia's (T-ALL). Oncogene 10, 1353-1360 (1995).
84. Oram, S. H. et al. A previously unrecognized promoter of LMO2 forms part of a transcriptional regulatory circuit mediating LMO2 expression in a subset of T-acute lymphoblastic leukaemia patients. Oncogene 29, 5796-5808 (2010).
85. Boehm, T. et al. An unusual structure of a putative T cell oncogene which allows production of similar proteins from distinct mRNAs. EMBO J 9, 857-868 (1990).
86. Smale, S. T. & Kadonaga, J.T . The RNA polymerase II core promoter. Annu Rev Biochem 72, 449-479 (2003).
87. Bernstein, B. E. et al. Genomic maps and comparative analysis of histone modifications in human and mouse. Cell 120, 169-181 (2005).
88. Wong, I. H. et al. Detection of aberrant p16 methylation in the plasma and serum of liver cancer patients. Cancer Res 59, 71-73 (1999).
89. Chim, S. S. et al. Detection of the placental epigenetic signature of the maspin gene in maternal plasma. Proc Natl Acad Sci U S A 102, 14753-14758 (2005).
90. Fernandez, A. F. et al. A DNA methylation fingerprint of 1628 human samples. Genome Res 22, 407-419 (2012).
91. Houseman, E. A. et al. DNA methylation arrays as surrogate measures of cell mixture distribution. BMC Bioinformatics 13, 86 (2012).
92. Chan, K. C. et al. Noninvasive detection of cancer-associated genome-wide hypomethylation and copy number aberrations by plasma DNA bisulfite sequencing. Proc Natl Acad Sci U S A 110, 18761-18768 (2013).
93. Lun, F. M. et al. Noninvasive prenatal methylomic analysis by genomewide bisulfite sequencing of maternal plasma DNA. Clin Chem 59, 1583-1594 (2013).
94. Ou, X. et al. Epigenome-wide DNA methylation assay reveals placental epigenetic markers for noninvasive fetal single-nucleotide polymorphism genotyping in maternal plasma. Transfusion 54, 2523-2533 (2014).
95. Jensen, T. J. et al. Whole genome bisulfite sequencing of cell-free DNA and its cellular contributors uncovers placenta hypomethylated domains. Genome Biol 16, 78 (2015).
96. Roadmap Epigenomics, C. et al. Integrative analysis of 111 reference human epigenomes. Nature 518, 317-330 (2015).
97. Visel, A. et al. ChIP-seq accurately predicts tissue-specific activity of enhancers. Nature 457, 854-858 (2009).
98. Koh, W. et al. Noninvasive in vivo monitoring of tissue-specific global gene expression in humans. Proc Natl Acad Sci U S A 111, 7361-7366 (2014).
99. Srinivasan, S. et al. Small RNA Sequencing across Diverse Biofluids Identifies Optimal Methods for exRNA Isolation. Cell 177, 446-462 e416 (2019).
100. Ibarra, A. et al. Non-invasive characterization of human bone marrow stimulation and reconstitution by cell-free messenger RNA sequencing. Nat Commun 11, 400 (2020).
101. Zhou, Z. et al. Extracellular RNA in a single droplet of human serum reflects physiologic and disease states. Proc Natl Acad Sci USA 116, 19200-19208 (2019).
102. Verwilt, J. et al. When DNA gets in the way: A cautionary note for DNA contamination in extracellular RNA-seq studies. Proc Natl Acad Sci USA 117, 18934-18936 (2020).
103. Adalsteinsson, V. A. et al. Scalable whole-exome sequencing of cell-free DNA reveals high concordance with metastatic tumors. Nat Commun 8, 1324 (2017).
104. Gentles, A. J. et al. The prognostic landscape of genes and infiltrating immune cells across human cancers. Nat Med 21, 938-945 (2015).
105. Binkley, M. S. et al. KEAP1/NFE2L2 Mutations Predict Lung Cancer Radiation Resistance That Can Be Targeted by Glutaminase Inhibition. Cancer Discov 10, 1826-1841 (2020).
106. Alig, S. et al. Short Diagnosis-to-Treatment Interval is associated with increased tumor burden measured by circulating tumor DNA and metabolic tumor volume in Diffuse Large B-cell Lymphoma. Journal of Clinical Oncology in press (2021).
107. Patro, R., Duggal, G., Love, M. I., Irizarry, R. A. & Kingsford, C. Salmon provides fast and bias-aware quantification of transcript expression. Nat Methods 14, 417-419 (2017).
108. Chen, S., Zhou, Y., Chen, Y. & Gu, J. fastp: an ultra-fast all-in-one FASTQ preprocessor. Bioinformatics 34, i884-i890 (2018).
109. George, J. et al. Comprehensive genomic profiles of small cell lung cancer. Nature 524, 47-53 (2015).
110. Newman, A. M. et al. Determining cell type abundance and expression from bulk tissues with digital cytometry. Nat Biotechnol 37, 773-782 (2019).

1-37. (canceled)

38. A method for determining fragment length diversity for a cell-free DNA molecules adjacent to at least one genomic site, the method comprising:
(i) obtaining a biological sample for analysis, the sample comprising circulating cell free DNA;
(ii) constructing a library from the cell free DNA;
(iii) sequencing components of the library; and
(iv) calculating, for each of the at least one genomic site, a measure of fragment length diversity for the sequenced library components that are within a threshold distance of the genomic site.

39. The method of claim 38, wherein the threshold distance is 1000 base pairs or less.

40. The method of claim 38, further comprising calculating nucleosome depleted region depth for each of the at least one genomic site.

41. The method of claim 38, further comprising determining a cell of origin of a cancer from an individual from whom the biological sample was obtained based on the calculated fragment length diversity measure for each of the at least one genomic site.

42. The method of claim 38, further comprising determining a tissue of origin of a cancer from an individual from whom the biological sample was obtained based on the calculated fragment length diversity measure for each of the at least one genomic site.

43. The method of claim 38, further comprising prior to sequencing components of the library:
hybridizing a selector to the library, wherein the selector is designed to enrich for cell-free DNA molecules within the threshold distance of the at least one genomic site; and
capturing the library components to which the selector was hybridized.

44. The method of claim 38, wherein the at least one genomic site comprises at least 10 genomic sites.

45. The method of claim 38, wherein the at least 10 genomic sites are transcription start sites.

46. The method of claim 45, wherein the transcription start sites are selected from transcription start sites set forth in Table 2.

47. The method of claim 43, wherein the selector is designed to enrich for cell-free DNA molecules from one or more of the ABC, GCB, positive control, negative control and DLBCL path categories of Table 2.

48. The method of claim 43, wherein the selector is designed to enrich for cell-free DNA molecules from one or more of the LUAD, LUSC, positive control and negative control categories.

49. The method of claim 43, wherein the selector is designed to enrich for cell-free DNA molecules within a threshold distance of a transcription start site of at least one of FOLR1_3, ITGA3_1, LRRC31_1, MACC1_1, NKX2-1_2, SCNN1A_2, SFTPB_2, WFDC2_1, CLDN1_1, FSCN1_1, GPC1_1, KRT17_1, PFN2_1, PKP1_1, S100A2_1, SFN_1, SOX2_2, TP63_2.

50. The method of claim 43, wherein the selector is designed to enrich for cell-free DNA molecules within a threshold distance of a transcription start site of MS4A1.

51. The method of claim 50, further comprising treating a subject from whom the biological sample was obtained with a therapy targeting CD20.

52. The method of claim 38, wherein the biological sample is obtained from an individual with cancer.

53. The method of claim 52 wherein the cancer is non-small cell lung carcinoma, small cell lung carcinoma, adenocarcinoma, squamous cell carcinoma, diffuse large B-cell lymphoma hepatocarcinoma, basal cell carcinoma, lymphoma, or melanoma.

54. The method of claim 38, wherein the circulating cell-free DNA sample is obtained prior to immune checkpoint inhibitor treatment.

55. The method of claim 38, wherein the circulating cell-free DNA sample is obtained within 4 weeks of a first immune checkpoint inhibitor treatment.

56. The method of claim 55, wherein the individual with cancer is treated with an immune checkpoint inhibitor if durable clinical benefit is predicted and treated with non-immune checkpoint inhibitor therapy if DCB is not predicted.

57. The method of claim 54, wherein the immune checkpoint inhibitor is a PD-1 or PD-L1 inhibitor.

58. The method of claim 38, wherein the sequencing is at a depth of 500× or greater.

59. The method of claim 38, wherein the sequencing is at a depth of 2000× or greater.

60. The method of claim 42, wherein the selector is designed to enrich for cell-free DNA molecules within a threshold distance of at least 50 transcription start sites in Table 2.

61. The method of claim 38, wherein the fragment length diversity measure is promoter fragment entropy is calculated using the equation PFE(TSS):=Eₖ[Σ_{i:1-5}P*(e_{TSS}>(1+k)×eᵢ)].

62. A method for treating a subject having one or more cancers, the method comprising:
obtaining a score based on a fragment length diversity measure for cell-free nucleic acid molecules within a threshold distance from a genomic site; and
when the score is lower than a predetermined value, administering an immune checkpoint inhibitor (101) to the subject, or
when the score is higher than a predetermined value, administering a therapy that is not an immune checkpoint inhibitor to the subject.

63. The method of claim 62, wherein the fragment length diversity measure is promoter fragment entropy.

64. The method of claim 62, wherein the genomic site is a transcriptional start site of a gene of interest.

65. The method of claim 38, wherein the genomic site is a site that differs in chromatin conformation among different cell types or states.
